(19) United States
(12) Patent Application Publication
(10) Pub. No.: US 2004/0126882 A1
(43) Pub. Date: Jul. 1, 2004

(54) Regulatable, catalytically active nucleic acids

(76) Inventors: Andrew D. Ellington, Austin, TX (US); Jay Hesselberth, Seattle, WA (US); Kristin Thompson, Arlington, MA (US); Michael P. Robertson, Santa Cruz, CA (US); Letha Sooter, Austin, TX (US); Eric Davidson, Houston, TX (US); J. Colin Cox, Austin, TX (US); Timothy Riedel, New Braunfels, TX (US); Charles Wilson, Concord, MA (US); Sharon T. Cload, Cambridge, MA (US); Anthony D. Keefe, Cambridge, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/254,568

(22) Filed: Sep. 24, 2002

Related U.S. Application Data
(63) Continuation-in-part of application No. 09/661,658, filed on Sep. 14, 2000.
(63) Continuation-in-part of application No. 09/883,119, filed on Jun. 14, 2001.
(63) Continuation-in-part of application No. 09/666,870, filed on Sep. 20, 2000.
(60) Provisional application No. 60/324,715, filed on Sep. 24, 2001.
(60) Provisional application No. 60/212,097, filed on Jun. 15, 2000.
(60) Provisional application No. 60/212,097, filed on Jun. 15, 2000.

(51) Int. Cl. C07H 21/02; C12N 15/85

(52) U.S. Cl. 435/455; 536/23.1

(57) ABSTRACT

Compositions and methods are provided to make, isolate, characterize and use regulatable, catalytically active nucleic acids (RCANA). The present invention is directed to RCANA that transduce molecular recognition into catalysis. Also, RCANAs according to the invention can be used as regulatory elements to control the expression of one or more genes in a metabolic pathway. RCANAs can also be used as regulated selectable markers to create a selective pressure favoring (or disfavoring) production of a targeted bioproduct.

### RELATED APPLICATIONS

This application claims the benefit of U.S. Ser. No. 60/324,715, filed Sep. 24, 2001; and is a continuation in part of U.S. Ser. No. 09/661,658, filed Sep. 14, 2000, which claims the benefit of U.S. Ser. No. 60/212,097, filed Jun. 15, 2000; and is a continuation in part of U.S. Ser. No. 09/666,870, filed Sep. 20, 2000, which claims the benefit of U.S. Ser. No. 60/212,097, filed Jun. 15, 2000; and is a continuation in part of U.S. Ser. No. 09/883,119, filed Jun. 14, 2001, which claims the benefit of U.S. Ser. No. 60/212,097, filed Jun. 15, 2000, each of which is incorporated herein by reference in its entirety.

### FIELD OF THE INVENTION

The present invention relates generally to the field of catalytic nucleic acids and in particular to regulatable, catalytically active nucleic acids that modulate their kinetic parameters in response to the presence of an effector.

### BACKGROUND OF THE INVENTION

Dyes, vitamins, food/chemical additives, enzymes, protein pharmaceuticals, pesticides, insecticides, and feed compounds for industrial chemical processes are but a few categories of compounds (bioproducts) derived from biological hosts. Many compounds in use or under development as therapeutics are synthesized entirely or partially in biological hosts, e.g., drugs such as antibiotics, anticancer drugs, antiftingals, cholesterol-lowering drugs, and immunosuppressants. In addition, a range of waste products (e.g., heavy metals) are actively accumulated within biological hosts in some bioremediation applications. Biological hosts used to synthesize/accumulate bioproducts include bacteria, eukaryotic microorganisms (e.g., Saccharomyces cerevisiae), plants, animal cells (e.g., transformed insect cells growing in culture), and animals.

The production of bioproducts can be high because many are generated at relatively low levels in host expression systems. To optimize expression of desired compounds that are synthesized or accumulated in biological host cells/organisms, careful control over the biosynthetic process is often required. Expression levels of a natural product can vary between related strains of a biological host.

Ribozymes are oligonucleotides of RNA that can act like enzymes and are sometimes called RNA enzymes. Generally, they have the ability to behave like an endoribonuclease, catalyzing the cleavage of RNA molecules. The location of the cleavage site is highly sequence specific, approaching the sequence specificity of DNA restriction endonuclcases. By varying conditions, ribozymes can also act as polymerases or dephosphoryl ases.

Ribozymes were first described in connection with Tetrahymena thermophilia. The Tetrahymena rRNA was shown to contain an intervening sequence (IVS) capable of excising itself out of a large ribosomal RNA precursor. The IVS is a catalytic RNA molecule that mediates self-splicing out of a precursor, whereupon it converts itself into a circular form. The Tetrahymena IVS is more commonly known now as the Group I Intron. A subclass of ribozymes are the catalytically active, regulatable nucleic acids (RCANAs). The catalytic activity of the RCANAs can be regulated by an effector. Effector-sensitive RCANAs have been described, wherein the activity of the RCANA is regulated by a ligand-binding moiety. Upon binding the ligand, the RCANA activity on a target RNA is changed. Such RCANAs have only been described for small molecule ligands such as organic or inorganic molecules. Effector-sensitive RCANAs can also be controlled by proteins, peptides, or other macro-molecules.

The invention makes it possible to rapidly optimize the production of useful bioproducts by identifying optimal conditions for production, by engineering the metabolic pathways of host cells/organisms used in biosynthesis/bioremediation, and by identifying genetic variants with improved biosynthetic/bioremediative properties.

### SUMMARY OF THE INVENTION

The present invention includes RCANAs wherein the catalytic activity of the RCANA is regulated by an effector. The RCANA of the present invention are, therefore, regulatable in that their activity is under the control of a second portion of the RCANA. Just as allosteric protein enzymes undergo a change in their kinetic parameters or of their enzymatic activity in response to interactions with an effector, the catalytic abilities of the RCANA may similarly be modulated by the effector(s). Thus, the present invention is directed to RCANA that transduce molecular recognition into catalysis. Also, RCANAs according to the invention can be used as regulatory elements to control the expression of one or more genes in a metabolic pathway. RCANAs can also be used as regulated selectable markers to create a selective pressure favoring (or disfavoring) production of a targeted bioproduct.

As will become apparent below, RCANA are more robust than allosteric protein enzymes in several ways: (1) they can be selected in vitro, which facilitates the engineering of particular constructs; (2) the levels of catalytic modulation are much greater for RCANA than for protein enzymes; and (3) since RCANA are nucleic acids, they can potentially interact with the genetic machinery in ways that protein molecules may not.

It should be noted that the methods described herein may include any type of nucleic acid. For example, these methods are not limited to RNA-based RCANA, but also encompass DNA RCANA and RNA or DNA RCANA. Furthermore, the methods can be applied to any catalytic activity the ribozymes are capable of carrying out. For example, the methods are not limited to ligases or splicing reactions, but could also encompass other ribozyme classes. The methods are also not limited to protein or peptide ligands, but also include-other molecular species, such as ions, small molecules, organic molecules, metabolites, sugars and carbohydrates, lipids and nucleic acids. The methods may also be extended to effectors that are not molecules, such as heat or light or electromagnetic fields. Furthermore, the methods are not limited to ligand-induced conformational. changes, but could also take into account chimeric catalysts in which residues essential for chemical reactivity were provided by both the nucleic acid and the ligand, in concert.

The effector may be a peptide, a polypeptide, a polypeptide complex, or a modified polypeptide or peptide. The effector may even be, e.g., an enzyme or even light (such as visible light) or even a magnet. The effector may be activated by a second effector that acts on the first effector (also referred to herein as an effector-effector), which may be an inorganic or an organic molecule. The polypeptide, peptide or polypeptide complex can be either endogenous, i.e., derived from the same cell type as the polynucleotide, or exogenous, i.e., derived from a cell type different than the cell from which the polynucleotide is derived.

The polypeptide or peptide may be phosphorylated or dephosphorylated. Alternatively, the effector may include a pharmaceutical agent. In some embodiments, the nucleic acid catalyzes a reaction that causes the expression of a target gene to be upregulated. In other embodiments, the nucleic acid catalyzes a reaction that causes the expression of a target gene to be down-regulated. If desired, the nucleic acid may be used to detect at least one exogenous effector from a library of candidate exogenous effector molecules. In some embodiments, the nucleic acid and the effector form a nucleic acid-effector complex.

In some embodiments, the kinetic parameters of nucleic acid catalysis are altered in the presence of a supermolecular structure, e.g., a viral particle or a cell wall. The nucleic acid may further include a regulatory element that can recognize a target molecule of interest. The nucleic acid may in addition include a transducer element that transmits information from the regulatory element to the catalytically active region of the nucleic acid.

Modification of Catalytic Residiues of RCANA. In one embodiment of this invention, the RCANA is generated by the modification of at least one catalytic residue. One of the unique features of the present selection protocol relative to others that have previously been published is that the present invention randomizes not only a domain that is pendant on the catalytic core, but a portion of the catalytic core itself. Thus, the selection for ligand-dependence not only yields species that bind to ancillary regions of the RCANA, but that may help stabilize the catalytic core of the RCANA.

Also provided by the invention is a method of isolating a regulatable, catalytically active nucleic, acid created by randomizing at least one nucleotide in the catalytic domain of a catalytically active nucleic acid to create a nucleic acid pool. The nucleic acid pool whose nucleic, acids interact with the catalytic target of the catalytic domain are removed. The method further may also include the step of adding an effector to the remaining pool of nucleic acids. In some embodiments, the method may also include the step of adding 4 an effector to the remaining nucleic acids, wherein the effector acts on the nucleic acids to alter the catalytic activities of the nucleic acids. The method may include optionally the step of purifying the isolated nucleic acid, and, if desired the step of sequencing the isolated nucleic acid. In various embodiments, the step of removing the nucleic acids is under high stringency conditions, moderate stringency conditions, or low stringency conditions.

In vitro sensing (or detection) applications. The current invention also provides for the use of RCANA for detection of a wide variety molecular species in vitro. For example, RCANA may be anchored to a chip, such as wells in a multi-well plate. Mixtures of analytes and fluorescently tagged substrates are added to each well. Where cognate effectors are present, the RCANA will covalently attach the fluorescent tags to themselves. Where RCANA have not been activated by effectors, the tagged substrates are washed out of the well. After reaction and washing, the presence and amounts of co-immobilized fluorescent tags are indicative of amounts of ligands that were present during the reaction. The reporter may be a fluorescent tag, but it may also be an enzyme, a magnetic particle, or any number of detectible particles. Additionally, the RCANA may be immobilized on beads, but they could also be directly attached to a solid support via covalent bonds.

One advantage of this embodiment is that covalent immobilization of reporters (as opposed to non covalent immobilization, as in EL1SA assays) allows stringent wash steps to be employed. Additionally, ribozyme ligases have the unique property of being able to transduce effectors into templates that may be amplified, affording an additional boost in signal prior to detection.

Modified nucleotides may be introduced into the RCANA that substantially stabilize them from degradation in environments such as sera or urine. The analytical methods of the present invention do not rely on binding per se, but only on transient interactions. The present invention requires mere recognition rather that actual binding, providing a potential advantage of RCANA over antibodies. That is, even low affinities are sufficient for activation and subsequent detection, especially if individual immobilized RCANA are examined (i.e., by ligand-dependent immobilization of a quantum dot).

Expression of RCANA in cells. The RCANAs of the present invention may also be expressed inside cells. The RCANAs of the present invention that are expressed inside a cell are not only responsive to a given effector, but are also able to participate in genetic regulation or responsiveness. In particular, self-splicing introns can splice themselves out of genes in response to exogenous or endogenous effector molecules.

The present invention includes RCANA constructs that may be inserted into a gene of interest, e.g, a gene targeting expression vector. The RCANA sequence provides gene specific recognition as well as modulation of the RCANA's kinetic parameters. The kinetic parameters of the RCANA vary in response to an effector. Specifically, in the case of RCANA that performs self splicing in the presence of the effector, the RCANA may splices itself out of the gene in response to the effector to regulate expression of the gene. RCANAs according to the invention can be used as regulatory elements to control the expression of one or more genes in a metabolic pathway. RCANAs can also be used as regulated selectable markers to create a selective pressure favoring (or disfavoring) production of a targeted bioproduct.

In another aspect, the invention includes a method of modulating expression of a nucleic acid by providing a polynucleotide that is regulated by a peptide. The polynucleotide may be a regulatable, catalytically active polynucleotide, in which the peptide interacts with the polynucleotide to affect its catalytic activity. The polynucleotide is contacted with the peptide, thereby modulating expression of a nucleic acid. The polynucleotide may be provided in a cell, and the cell may be, e.g, provided in vitro or in vivo and may be a prokaryotic cell or a even a eukaryotic cell.

The present invention also includes an RCANA construct with a regulatable oligonucleotide sequence having a regulatory domain, such that the kinetic parameters of the RCANA on a target gene vary in response to the interaction of an effector with the regulatory domain.

In vivo sensing (or detection) applications. It is possible to activate or repress a reporter gene (e.g., luciferase) containing an engineered intron in response to an endogenous activator. In this way, luciferase-engineered intron constructs may be used to monitor intracellular levels of proteins or small molecules such as cyclic AMP. This method may be used for in vivo measurements in both cellular systems, such as cell culture, and in whole organisms, such as animal models. Such applications may be used for high-throughput screening. If a particular intracellular signal (e.g, the production of a tumor repressor) was desired, compound libraries for pharmacophores that induce the signal (the tumor repressor) are screened for activation of the reporter gene. Thus, the information desired is changed or morphed into the detection of glowing cells.

Gene therapy applications. Similarly, a gene can be activated or repressed in response to an exogenously introduced effector (drug) for gene therapy. The RCANA may be used for gene expression up regulation (increasing production of the gene product) or down regulation (decreasing the production of the gene product). The construct of one embodiment of the present invention provides a DNA oligonucleotide coding for a catalytic domain and effector binding domain. The advantages of the nucleic acid-based technology of the present invention include, e.g., the ability to continually modulate gene expression with a high degree of sensitivity without additional gene therapy interventions.

In another aspect, the invention includes a method of modulating expression of a nucleic acid in a cell by providing a polynucleotide that is regulated by an effector, e.g., a peptide. The polynucleotide may be a regulatable, catalytically active polynucleotide, in which the peptide interacts with the polynucleotide to affect its catalytic activity. The polynucleotide is contacted with the peptide, thereby modulating expression of a nucleic acid. The polynucleotide may be provided in a cell, and the cell may be, e.g., provided in vitro or in vivo and may be a prokaryotic cell or a even a eukaryotic cell.

Biosynthelic application of RCANAs. This invention describes methods by which effector-sensitive RCANAs can be used to facilitate industrial biosynthesis and bioremediation. For example, provided are methods in which effector-dependent ribozymes can be used to (1) control production of a natural product in a biological host, (2) to identify environmental conditions which increase biosynthetic yields, and (3) to isolate strains of a biological host with improved product yields and/or properties.

As sensors, effector-sensitive RCANAs can be used to accurately monitor the concentration of a natural product as it is produced, either directly in vivo or ex vivo (e.g. following lysis). A wide range of environmental conditions and variant strains can be tested for synthesis of a natural product and the riboreporter used to define the best conditions/variants to use for production. Sensor applications include both in vivo and in vitro applications.

As regulatory elements, effector-sensitive RCANAs can be used to control the expression of one or more genes involved in a metabolic pathway. By coordinating gene expression, it is possible to optimize production of a targeted metabolite, staging production of the intermediates required to generate it or timing induction of bioproduct synthesis with respect to host growth.

As regulated selectable markers, effector-sensitive RCANAs can be used to create a selective pressure favoring (or disfavoring) production of targeted bioproduct. Host strains carrying the RCANA-selectable marker construct can be subjected to mutagenesis or transformed with a vector library in ways that change the concentration of bioproduct generated by the host. When subjected to appropriate selection conditions, variants with the highest (or lowest) internal concentrations of the targeted bioproduct are favored for survival.

The present invention includes, methods of regulating (e.g., increase or decrease) production of a product in a cell. Production is regulated by inserting into a gene that produces the product or alternatively regulates the production of the product a RCANA. An RCANA includes a catalytic domain and a regulatory (or effector) domain. The regulatory domain is contacted with an effector such as to alter (i.e., activate) a function of the catalytic domain.

Also provided by the invention are methods of regulating a biological pathway in a cell, by inserting into two or more genes that produce a product or regulates the production of the product in the biological pathway in a cell a RCANA. The regulatory domain of the RCANA is contacted with an effector such as to alter (i.e., activate) a function of the catalytic domain. For example, a biological pathway is regulated in a cell, by inserting into a first gene that produces a first product or regulates the production of the first product in the biological pathway in a cell a first RCANA. Following insertion of the first RCANA, an RCANA is inserted into a second gene that produces a second product or regulates the production of the second product in the biological pathway in the cell. The first effector activating the first RCANA is then contacted with the first regulatory domain thereby regulating production of the first product and the second regulatory domain is contacted with the second effector thereby regulating production of the second product. The first product can be the second effector.

The first and second RCANAs can block expression of the first and second gene. Alternatively, the first and second RCANAs can activate expression of the first and second gene. Accordingly, the combination of the first and second effectors can control the flux of metabolites through a biological pathway.

In various embodiments, the above method can be further elaborated by inserting an RCANA into a third gene that produces a third product or regulates the production of a third product. RCANAs can be inserted into additional genes (e.g., four, five six or more genes) to further regulate the biological pathway.

The biological pathway can be for example a biosynthetic pathway. The biological pathway can a metabolic pathway. The biological pathway can be either fully inhibited or partially inhibited according to the concentration of the first and second effectors.

By catalytic domain is meant that the domain modifies a transcript to alter its coding potential, i.e., the ability to be transcribed or translated to yield the encoded polypeptide. Modification includes splicing, e.g., self splicing and rescission and ligation, and endonucleoltyic cleavage. Modification can be non-covalent, e.g., structural or conformational alteration or a covalent modification.

By regulatory domain is meant the region of effector binding on an RCANA.

Function of the catalytic domain include, endonucleolytic and ligase activity. For example the catalytic domain catalyzes cleavage of the RCANA. Alternatively the catalytic domain catalyzes the excision of the RCANA from the gene in which it is inserted followed by ligation of the gene at the 5′ and 3′ ends of the cleavage site.

The product is the endproduct of a biosynthetic/metabolic process. For example, the product is a protein, an enzyme, a protein pharmaceutical, a metabolite, a drug, a dye, a vitamin, a food additive, a chemical additive, a pesticide, an insecticide, or a feed compound.

The effector is a substance other than the target product or it can be the target product. The effector is a protein, an enzyme, a protein pharmaceutical, a metabolite, a drug, a dye, a vitamin, a food additive, a chemical additive, a pesticide, an insecticide, a feed compound, or a waste product. Where the effector is a drug, the effector can be an antibiotic, anticancer drug, antifungal, cholesterol-lowering drug, or immunosuppressant.

For example, where the effector is the product, it can also act as a feedback inhibitor of the gene in which the RCANA is inserted or be an intermediate in a biosynthetic/metabolic pathway. The effector is either exogenous or endogenous to the cell. Contacting the regulatory domain with the effector(s) may either increase or decrease production of the targeted product compared to the level of target product observed in the absence of effector(s) (i.e., normal control level). The effector(s) may mediate RCANA activity in a concentration-dependent manner.

The RCANA blocks or activates the expression of the gene. By blocks expression is meant that the RCANA interrupts the transcription of the gene or the translation of the protein. By activates expression is meant that the effector binding of the RCANA leads to enhanced transcription of the gene or the translation of the protein.

The effector may act via a feedback mechanism to regulate the activity of the target gene. That is, an effector-activated RCANA can act in a negative feedback loop to inhibit the target gene. Alternatively, an effector-activated RCANA can act in a positive feeback loop to increase expression of the target gene.

The cell is for example a prokaryotic eukaryotic, bacterial, or insect cell. In yet another aspect, the present invention includes, methods of screening a population of cells for a cell that produces a bioproduct. This screening method includes inserting an RCANA into a reporter gene, e.g., green fluorescent protein, thymidylate synthase, or beta lactamase in a population of cells, such that the RCANA is regulated by the bioproduct. The product of the reporter gene provides a growth advantage to host cells expressing the bioproduct. Cells producing the bioproduct can be isolated by measuring the expression of the reporter gene which indicates the production of the bioproduct in the cell. Cell are isolated by methods know in the art such a fluorescent activated cell sorting.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

For a more complete understanding of the features and advantages of the present invention, reference is now made to the detailed description of the invention along with the accompanying figures in which corresponding numerals in different figures refer to corresponding parts and in which:

FIG. 1 is a depiction of the secondary structure of the Group I theophylline-dependent (td) intron of bacteriophage T4 (wild type).

FIG. 2a is a photograph of a gel showing activation of the GpITh1P6.131 aptamer construct, together with a graphical representation of the gel, of one embodiment of the present invention; FIG. 2b is a photograph of a gel showing activation of GpITh2P6.133 aptamer construct, together with a graphical representation of the gel of one embodiment of the present invention.

FIG. 3 is a schematic depiction of an in vivo assay system for group I introns of one embodiment of the present invention.

FIG. 4a is a depiction of a portion of the P6 region of the Group I ribozyme joined to the anti-theophylline aptamer by a short randomized region to generate a pool of aptazymes of the present invention.

FIG. 4b is a schematic depiction of a selection protocol for the Group I P6 Aptazyme Pool of FIG. 4a.

FIG. 5 is a diagram of one embodiment of the present invention depicting exogenous or endogenous activation of Group I intron splicing;

FIG. 6 is a diagram of another embodiment of the present invention depicting a strategy for screening libraries of exogenous activators;

FIG. 7 is a diagram of an alternative embodiment of the present invention for screening libraries of exogenous activators;

FIG. 8 is a diagram of yet another alternative embodiment of the present invention for screening libraries of exogenous activators;

FIG. 9 is a diagram of an embodiment of the present invention for screening for endogenous activators;

FIG. 10 is a diagram of an alternative to the embodiment of FIG. 9 of the present invention to screen for endogenous activators;

FIG. 11 is a diagram of another embodiment of the present invention to screen for compounds that perturb cellular metabolism;

FIG. 12 is a diagram of a further embodiment of the present invention that provides a non-invasive readout of metabolic states;

FIG. 13 is a diagram of yet a further embodiment of the present invention wherein endogenous suppressors provide a non-invasive readout of multiple metabolic states;

FIG. 14 is a schematic depiction of an example of a work surface for automatic selection procedures of one embodiment of the invention;

FIG. 15a is an illustration of the L1 ligase aptazyme construct of one embodiment of the present invention; FIG. 15b is an illustration of a modified L1 ligase aptazyme construct of FIG. 15a of the present invention;

FIG. 15c is a schematic diagram of a selection protocol of one embodiment of the present invention;

FIG. 16(a-c) is a schematic diagram of a method to anchor an aptazyme construct of the present invention to a solid support in one embodiment of the present invention;

FIGS. 17(a-c) is a schematic showing the L1 ligase was the starting point for pool design;

FIG. 18(a-d) are charts and graphs showing the progression of the L1-N50 selections;

FIG. 19(a & b) is a schematic showing protein-dependent regulatable, catalytically active nucleic acid sequences and structures;

FIG. 20 is a graph demonstrating the ribozyme activity with inactivated protein samples;

FIG. 21 is a graph demonstrating an aptamer competition assays;

FIG. 22 is a flow chart of a method for negative and positive selection of RCANA;

FIG. 23 is a flow chart of a method for negative and positive selection of RCANA;

FIG. 24 is a graph showing the progress of the L1-N50 Rev selection;

FIG. 25(a,b,c) schematic showing the theophylline-dependent td group I intron constructs of the present invention;

FIG. 26 is a schematic showing the design of an FMN-dependent td nucleic acid intron splicing construct;

FIGS. 27(a-c) is a graph showing the relative growth curves of theophylline-dependent in vivo growth;

FIG. 28 is a graph showing 3-Methylxanthine dependent in vivo growth;

FIG. 29(a & b) is a schematic of a ribozyme ligase array;

FIG. 30 is an image showing the results of a rcgulatable, catalytically active ligase array;

FIG. 31 is a graph showing the titrations of individual allosteric ribozyme ligases;

FIG. 32 is a schematic showing RCANA-mediated control of gene expression in a biochemical metabolic pathway using a single exogenous effector;

FIG. 33 is a schematic showing RCANA-mediated control of gene expression in a biochemical metabolic pathway using multiple exogenous effectors;

FIG. 34 schematic showing shows the usc of metabolite-sensitive RCANAs to maintain constant levels of end-product in a biochemical metabolic pathway;

FIG. 35 is a schematic showing the use of RCANA-mediated control of gene expression;

FIG. 36 is a schematic showing RCANA-based cell selection;

FIG. 37 is a schematic showing RCANA-based cell selection;

FIG. 38 is a schematic showing RCANA- based in vivo sensors;

FIG. 39 is a schematic showing synthesis of an RCANA-based in vivo sensor.

### DETAILED DESCRIPTION OF THE INVENTION

While the making and using of various embodiments of the present invention are discussed in detail below, the present invention provides many applicable inventive concepts that may be embodied in a wide variety of specific contexts. The specific embodiments discussed herein are merely illustrative of specific ways to make and use the invention and do not delimit the scope of the invention.

The present invention includes compositions or matter, methods and automation that permit the creation, isolation, identification, characterization and optimization of regulatable catalytically active nucleic acids. Furthermore, it includes methods to use RCANA for in vitro sensing (or detection), in vivo sensing (or detection), and gene therapy. Regulatable, catalytically active nucleic, acids selected by the method of the present invention also have advantages over other biopolymers that might be used for sensing or gene regulation. Regulatable, catalytically active nucleic acids are more robust than allosteric protein enzymes in several ways: (1) they can be selected in vitro (facilitating the engineering of particular constructs); (2) the levels of catalytic modulation arc much greater than those typically observed with protein enzymes; and (3) since regulatable, catalytically active micleic acids are nucleic acids, they can potentially interact with the genetic machinery in ways that protein molecules may not.

The method is not limited to RNA pools, but may also encompass DNA pools or modified RNA pools. Modified nucleotides may be introduced into the regulatable, catalytically active nucleic acids that substantially stabilize them from degradation in environments such as sera or urine. The method is not limited to ligascs, but could also encompass other ribozyme classes. The method is not limited to protein-induced conformational changes, but could also take into account chimeric catalysts in which residues essential for chemical reactivity were provided by both the nucleic acid and the protein in concert. Initially, many protein targets may prove refractive to selection. Many derivatives of the base method can be developed, however, to deal with novel targets or target classes.

A. Protein Dependent RCANA

Effector-dependent ribozymes have been shown to be responsive to small organic compounds, such as ATP and theophylline. The present inventors recognized the need for effector-dependent ribozymes, or as used herein, “regulatable, catalytically active nucleic acids” that are responsive to larger molecules, such as, e.g., peptides or proteins. The peptides, proteins or other large molecules may be provided from endogenous sources (e.g., expressed within a cell or cell extract), or exogenous sources (added or expressed in a cell or cell extract).

In order to understand the present invention, it is important to understand that previous attempts to make catalytically active nucleic acids that interact and respond to a large effector, by the inventors and others, have failed. Initially, attempts were made to generate protein-dependent ribozymes by the addition of aptamers (known binding sequences) to ribozymes (catalytically active domains). This design and method was unsuccessful in providing regulatable nucleic acids. Next, attempts were made to generate protein-dependent ribozymes by adding random sequence regions between an aptamer (binding) and a ribozyme (catalytic) and selecting for effector-dependence. These attempts were also unsuccessful. Next, the inventors attempted to generate protein-dependent ribozymes by adding a large random sequence region to the catalytic cores of ribozymes and selecting for effector-dependence. These attempts were also unsuccessful. In other words, all previously detailed methods for the generation of ribozymes that were dependent on small organic compounds were unsuccessful for generating ribozymes that were dependent on proteins.

To date, the present inventors have selected a number of protein-and peptide dependent ribozyme ligases. One example is the isolation of a protein-dependent, regulatable, catalytically active nucleic acid with an activity that was increased in a standard assay by 75,000-fold in the presence of its cognate protein effector, tyrosyl tRNA synthetase from Neurospora mitochondria (Cyt18). The Cyt18-dependent ribozyme was not activated by non-cognate proteins, including other tRNA synthetases.

A protein-dependent regulatable, catalytically active nucleic acid was also created and selected with an activity that was increased by 3,500-fold in the presence of its cognate protein effector, hen egg white lysozyme. The lysozyme-dependent ribozyme was not activated by most non-cognate proteins, including T4 lysozyme, but was activated by a very closely related protein, turkey egg white lysozyme. Moreover, the protein-dependent ribozyme was inhibited by a RNA binding species that specifically bound to lysozyme. In other words, the activation of these protein-dependent ribozymes was highly specific.

A peptide-dependent, regulatable, catalytically active nucleic acids was also created and isolated with activity was increased by 18,000-fold in the presence of its cognate peptide effector, the arginine-rich motif (ARM) from the HIV-I Rev protein. The Rev-dependent nucleic acid was not activated by other ARMs from other viral proteins, such as UTLV-I Rex. Using the present invention, regulatable, catalytically active nucleic acids may be developed that are regulated by any of a vast number of effectors.

As will be clear from the continued description, protein dependent RCANAs are useful in a variety of applications. For example, protein-regulated catalytically active nucleic acids can be used (1) for the acquisition of data about whole proteomes, (2) as in vitro diagnostic reagents to detect proteins specific to disease states, such as prostate specific antigen or viral proteins, (3) as sentinels for the detection of biological warfare agents, or (4) as regulatory elements in gene therapies.

### B. Modification of Residues in Catalytic Domain

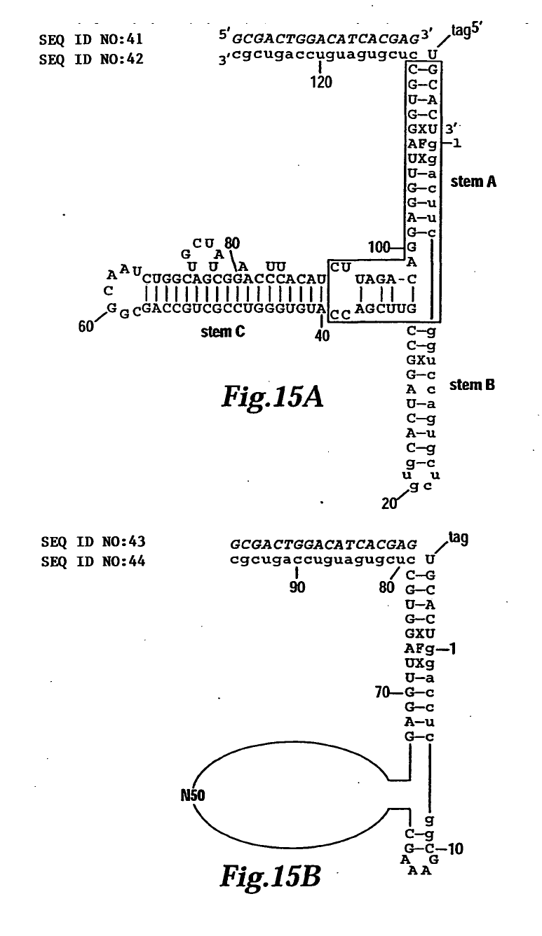

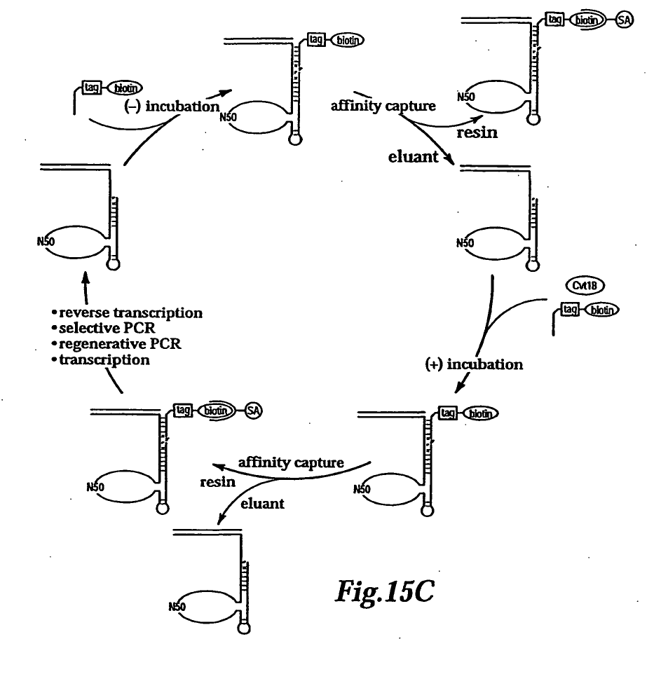

In one embodiment, the present invention randomizes a portion of the catalytic core itself, not necessarily a domain that is pendant on the catalytic core. One example for selection using the present invention was using the L1 ligase. The catalytic core of the L1 ligase has been mapped by deletion analysis and by partial randomization and re-selection. FIG. 15a depicts the L1 ligase that was the starting point for pool design. Stems A, B, and C are indicated. The shaded region contains the catalytic core and ligation junction. Primer binding sites are shown in lower case, an oligonucleotide effector required for activity is shown in italics, and the ligation substrate is bolded. The ‘tag’ on the ligation substrate can be varied, but was biotin in the exemplary selection described herein. The L1 pool contains random sequence positions and overlaps with a portion of the ribozyme core. In FIG. 15b, Stem B was reduced in size and terminated with a stable GNRA tetraloop, but stem A was unchanged.

A pool was synthesized in which the random sequence region spanned the catalytic core. Protein-dependent ribozymes were selected from this random sequence pool by selecting for the ability to ligate an oligonucleotide tag in the presence of a protein effector followed by capturing the oligonucleotide tag on an affinity matrix, followed by amplification in vitro or in vivo. Because the catalytic core has been randomized, the selection for protein-dependence not only yields species that may bind to ancillary regions of the ribozyme, but species in which the protein effector actually helps to organize the catalytic core of the ribozyme.

Selection for protein-dependence from a pool in which at least a portion of the catalytic core of the ribozyme is randomized differs from selection for protein dependence from a pool in which the catalytic core is not randomized. For example, the catalytic core of the protein-dependent ribozymes that was selected differed substantially from the catalytic core of the original ribozyme and the catalytic core of other, nonprotein-dependent ribozymes selected based on the original ribozyme.

FIG. 15a depicts the L1 ligase that was the starting point for pool design in the Cyt18 RCANA selection, as an example of a protein-activated regulated, catalytically active nucleic acid. Stems A, B, and C are indicated. The shaded region contains the catalytic core and ligation junction. Primer binding sites are shown in lower case, an oligonucleotide effector required for activity is shown in italics, and the ligation substrate 14 is bold font. The ‘tag’ on the ligation substrate can be varied, but was biotin in the exemplary selection described herein. The LI pool contains 50 random sequence positions and overlaps with a portion of the ribozyme core. In FIG. 15b, Stem B was reduced in size and terminated with a stable GNRA tetraloop, but stem A was unchanged.

Because one or more residues in the catalytic core have been randomized, the effectors may add essential catalytic residues for a given reaction. That is, both the effector molecule and the regulatable, catalytically active nucleic acids contribute a portion of the active site of the ribozyme. For example, using the method of the present invention a ribozyme and an effector molecule that would only carry-out poorly an enzymatic function independently may perform that enzymatic function upon interaction with one another. As such, a regulatable, catalytically active nucleic acid that contributes a guanosine and an adenosine and a protein effector that contributes a histidine together form a complex that has greater activity than either of the individual compounds. Using the methods disclosed herein it is possible to identify a chimeric effector:ribozyme (e.g., a protein:RNA complex) active site that would lead to catalysis. The invention describes ribozymes that have a detectable, basal chemical reactivity, and that the presence of the effector modulates this basal chemical reactivity. It is for this reason that the present invention differs significantly from other inventions which have claimed protein:RNA complexes in which no basal catalytic activity exists in the ribozyme or protein alone.

### C. Selection of RCANA

FIG. 15c schematically shows the following selection scheme: the RNA pool was incubated with a biotinylated tag and reactive variants were removed from the population. The remaining species were again incubated with a biotinylated tag in the presence of the target (for example the protein Cyt18). Reactive variants were removed from the population and preferentially amplified by reverse transcription, PCR, and in vitro transcription.

Ligand-dependent, regulatable, catalytically active nucleic acids selected by this method differ from functional nucleic acids selected from random sequence pools. Selection for ligand-dependence requires a selection for catalytic activity as opposed to a selection for binding. Therefore, protein-dependent, regulatable, catalytically active nucleic acids are not aptamers. The composition of matter of a selected protein dependent ribozyme will be different than the composition of matter of a selected aptamer. For example, the sequence of the lysozyme-dependent ribozyme is different from the sequence of anti-lysozyme aptamers. An important feature of the present invention is that the regulatable, catalytically active nucleic acids disclosed herein only required recognition rather than selected or enhanced binding ability. For example, the affinity of lysozyme for the naive, unselected RNA pool is identical to the affinity of lysozyme for the selected, regulatable, catalytically active nucleic acid. The only difference is that the way in which lysozyme is recognized by the regulatable catalytically active nucleic acids leads to activation, while for the pool as a whole non-specific binding does not lead to activation. In other words, binding is a concomitant but secondary function of selection for regulatability; that is, the regulatable ribozymes disclosed herein may bind the effector or target very poorly, but upon interaction the activity of the ribozyme may nonetheless be modulated.

### D. Automated Selection of RCANA

Robotic workstations have become essential to high-throughput manipulations of biomolecules, such as in high-throughput screening for drugs with a particular mechanism of action. The invention also includes the automation of in vitro selection procedures, and a mechanized system that executes both common and modified in vitro selection procedures. Automation facilitates the execution of this procedure, accomplishing in h to days what once necessitated weeks to month. In particular, the present inventors have adapted a robotic workstation to the selection of aptamers and ribozymes. However, the automation methods are generalizable to a number of different types of selections, including selections with DNA or modified RNA, selections for ribozymes, and selections for phage-displayed or cell-surface proteins.

In short, in vitfro selection involves several components: generation of a random sequence pool, sieving the random sequence pool for nucleic acid species that bind a given target or catalyze a given reaction, amplification of the sieved species by a combination of reverse transcription, the polymerase chain reaction, and in vitro transcription. Beyond the generation of the random sequence pool, each of these steps can potentially be carried out by a robotic workstation. The pool can be pipetted together 16 with a target molecule. If the target is immobilized on a magnetic bead, then the nucleic acid:target complex can be sieved from solution using an integrated magnetic bead collector. Finally, selected nucleic acid species can be eluted from the complex and amplified via a series of enzymatic steps that include the polymerase chain reaction carried out via an integrated thermal cycler.

There are many potential ways in which binding species can be sieved fiom a random sequence population. However, not all of these methods are amenable to automated selection. For example, to select aptamers, others have suggested that targets can be immobilized onto microtitre plates and binding species can be sieved by panning. The present inventors have had little success with this method, likely because panning is a relatively inefficient, low stringency method for sieving. Instead, the present inventors have discovered that when targets are immobilized on beads and mixed with a random sequence pool, binding species can be efficiently sieved from non-binding species by filtration of the beads. Beads can be readily manipulated by pipetting, allowing for the facile recovery and elution of the binding species, which are then amplified and carried into subsequent rounds of selection. This method differs from the magnetic bead capture method, and can be carried out with much higher stringency. This method is novel, and has not previously been used for in vitro selection experiments.

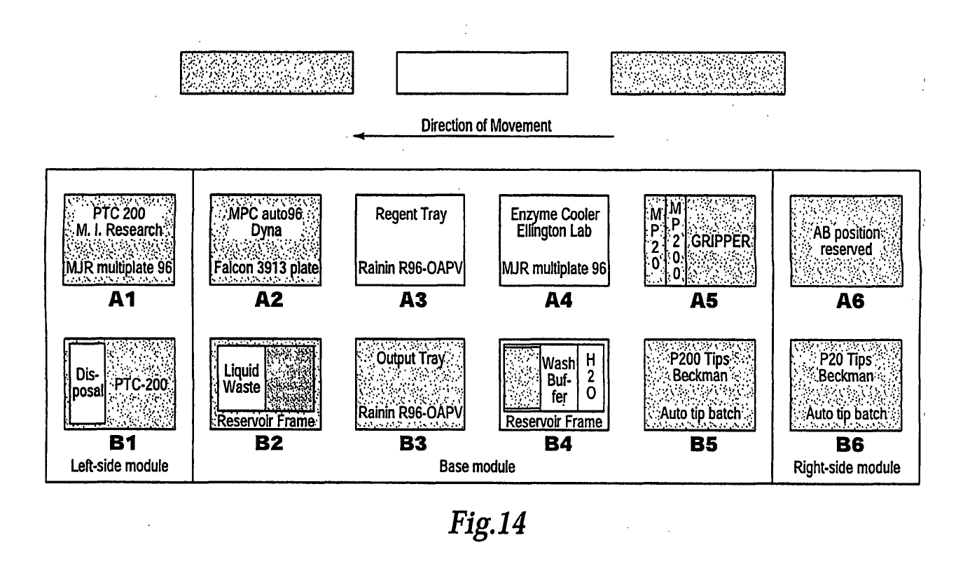

FIG. 14 depicts schematically an exemplary work surface for yet another embodiment of the present invention: automated selection. See, J. C. Cox, et al., Automated RNA Selection Biotechnol Prog., 14, 845 850, 1998.

Base protocol. Automated selection involves several, sequential automated steps.

Several modules are placed on the robotic work surface, including a magnetic bead separator, and enzyme cooler, and a thermal cycler. After manually preparing reagents and preloading those reagents (including random pool RNA, buffers, enzymes, streptavidin magnetic beads, and biotinylated target) and tips onto the robot, a program is run. The selection process, automated by the robot, goes as follows: RNA pool is incubated in the presence, of biotinylated target conjugated to streptavidin magnetic beads. After incubation, the magnets on the magnetic bead separator are raised, and the beads (now bound by pool RNA-the selected nucleic acids) are pulled out of solution. Thus, the beads can be washed, leaving only RNA bound to targets attached to beads. These RNA molecules are reverse transcribed, reamplified. via PCR, and the PCR DNA is in vitro transcribed into RNA to be used in iterative rounds of selection.

The Bioworks method for in vitro selection. This scripted programming method contains all movements necessary in order to facilitate automated selection. This includes all physical movements to be coordinated, and also communication statements. For instance, five rounds of automated selection against a single target requires over 5,000 separate movements in x, y, z, t coordinate space. Additionally, the method also holds all relevant measurements, offsets, and integrated equipment data necessary to prevent physical collisions and permit concerted communication between devices.

“Beads on filler” selections. While the vast majority of manual selections have been performed on nitrocellulose-based filters, a small few have also been performed on solid surfaces, such as beads. A novel selection scheme was developed whereby selection is performed on magnetic beads that are placed on nitrocellulose filters, and washed as the bead is the selection target itself. This method allows for much greater specificity of selection, thereby promoting ‘winning’ molecules to amplify in greater number, and thus reduce the overall amount of rounds necessary to complete the selection procedure. Manual selection does not involve a combination of surfaces to enhance selection. An alternative method is to take the magnetic beads, or nucleic acids attached to beads using methods other than beads, and running buffer over the beads and through a filter. It has been found that a complete filter washing step provides improved performance in the selection due to decreased background. One example of the automation of such a methods would be to remove, e.g., nucleic acids attached to the beads by placing the beads in a 96-well plate with a filtered bottom, the beads washed with buffer followed by subsequent elution of the target nucleic acids.

Cross-contamination avoidance. The introduction of contaminating species of nucleic acid strands in a manual selection may be commonplace. This is especially true if selection is done against multiple targets in parallel, and also when a researcher reuses the same pool for different selection tools. Contaminating species have been shown in the past to interfere with a manual selection such that it could not be completed. Automated in vitro selection takes steps to minimizing and/or eliminate the possibility of cross-contamination between pools and targets. Movement of the mechanical pod along the 18 work surface is unidirectional when carrying potentially contaminating material. This movement away from °Clean' things and only towards items that have already been exposed to replicons greatly diminishes the possibility of cross-contaminating reactions. The only circumstance in which the pod reverses its direction is to acquire a new, clean pipette tip. Additionally, the reagent trays were sealed with aluminum foil for a physical barrier between the environment and unexposed reagents. See FIG. 14, a layout of the robotic work surface that reduces cross-contamination.

Using this method the present inventors have successfully selected aptamers against a number of protein targets, including Cyt18, lysozyme, the signaling kinase MEKI, Rho from a thermophilic bacteria, and the Herpes virus US11 protein. The robot can perform 6 rounds of selection/day versus individual protein targets, and selections are typically completed within 12-18 rounds. In each instance, selected populations showed a substantially greater affinity for their cognate proteins than naive populations. In addition, when selected populations were sequenced one or more sequence families typically predominated. Sequence families are a hallmark of a successful selection, and indicate that the robotic method faithfully recapitulates manual selection methods.

The use of beads for target immobilization allows automated selection to be generalized to virtually any target class. For example, small organic molecules could be directly conjugated to beads. Similarly, antibodies could be conjugated to beads and in turn could be used to capture macromolecular structures, such as viruses or cells.

In another embodiment, the robotic workstation can be used for the selection of nucleic acid catalysts. For example, a DNA library was incubated that contained an iodine leaving group at its 5′ end with a DNA oligonucleotide substrate containing a 3′ phosphorothioate nucleophile and a 5′ biotin. The biotin can be captured on beads bearing streptavidin, and the beads can in turn be captured either by magnetic separation or by filtration. Any molecules in the DNA pool that ligate themselves to the biotinylated substrate are co-immobilized with that substrate. Immobilized species can be directly amplified following transfer to the integrated thermal cycler. The inclusion of a biotin on one of the primers used for amplification allows single-stranded DNA to be prepared by denaturation of the non-biotinylated strand in base, followed by neutralization of the solution. While this method has proved successful for the selection of deoxyribozyme 19 ligases, variations could also have been attempted. For example, the biotinylated DNA oligonucleotide substrate could have been pre-immobilized on beads, and the DNA pool incubated with the beads. In this instance, any molecules in the DNA pool that ligate themselves to the substrate will also be directly captured on the beads.

The use of beads for catalyst immobilization immediately suggests other selection protocols. For example, nucleic acid cleavases could be selected by first immobilizing a pool on the beads, then selecting for those species that cleave themselves away from the beads. Similarly, nucleic acid Diels-Alder synthetases may be selected by first immobilizing a diene on the beads, creating a nucleic acid pool that terminates in a dienophile, and selecting for those species that most efficiently con ugate the diene and dienophile.

This method can be applied to the selection of RCANAs. The ability to use a robotic workstation to select for ligases demonstrates that it is possible to select for regulatable ribozymes. For example, the selection protocols described in this invention can be altered so that ligases that immobilized themselves in the absence of a protein effector are removed from the random sequence population, while ligases that subsequently immobilized themselves once a protein effector were added are transferred to the integrated thermal cycler, amplified, and used for additional rounds of selection. This automated selection methods for regulatable ribozymes can readily be extended to other classes or catalysts than ligases, such as cleavases or Diels Alder synthetases by those skilled in the art.

Automating selection greatly diminishes human error in the actual pipetting and biological manipulations. While programming the robot is often not a trivial task, and can be time-consuming, automated selection is far faster and more efficient than manual selection. The scicntist's time is thus put to better use preparing samples and analyzing data, rather than performing the actual selection. Additionally, automated selection may include real-time monitoring methods (e.g., molecular beacons, TaqMan) and software that can make intelligent decisions based on real-time monitoring.

### E. Chip-based RCANA for in vitro Detection Applications

Regulatable catalytically active nucleic acids are especially useful for biosensor applications. For example, different protein-regulated catalytically active nucleic acids may be anchored to a surface, such as wells in a multi-well plate. Mixtures of analytes and fluorescently tagged substrates are added to each well. Where cognate effectors are present, the protein-regulated catalytically active nucleic acids will covalently attach the fluorescent tags to themselves. Where protein-regulated catalytically active nucleic acids have not been activated by effectors, the tagged substrates will be washed out of the well. After reaction and washing, the presence and amounts of co-immobilized fluorescent tags are indicative of amounts of ligands that were present during the reaction.

In one embodiment of the invention, the reporter may be a fluorescent tag, but it may also be an enzyme, a magnetic particle, or any number of detectable particles. Additionally, the protein-regulated catalytically active nucleic acids may be attached to beads or non-covalently linked to a surface rather than covalently linked to a surface.

One advantage of this method is that covalent immobilization of reporters (as opposed to non-covalent immobilization, as in ELISA assays) allows stringent wash steps to be employed. Additionally, ribozyme ligases have the unique property of being able to transduce effectors into nucleic acid templates that can be amplified, affording an additional boost in signal prior to detection.

Another advantage is that the analytical methods of the present invention do not rely on binding per se, but only on transient interactions. The present invention requires mere recognition rather than a binding event that must be physically isolated, providing a potential advantage of protein-regulated catalytically active nucleic acids over antibodies. That is, even low affinities are sufficient for activation and subsequent detection, especially if individual, immobilized protein-regulated catalytically active nucleic acids are examined (i.e., by ligand-dependent immobilization of a quantum dot).

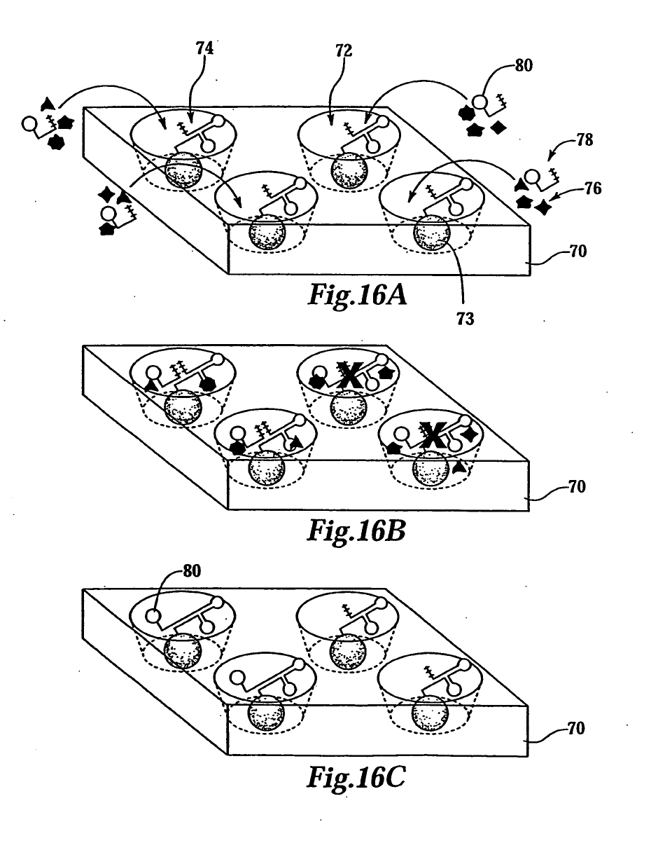

FIG. 16 schematically depicts one way to anchor aptazymes to a chip for a particular embodiment of the present invention. In this schematic, different ribozyme ligases (indicated by different colored allosteric sites) are shown immobilized on beads in wells, and mixtures of analytes (differentiated by shape and color) and fluorescently tagged substrates have been added to each well. In the middle panel of this figure, where 21 cognate effectors are present (same color analyte and allosteric site), the aptazymes will covalently attach the fluorescent tags to themselves. Where RCANA have not been activated by effectors, the tagged substrates are washed out of the well. In the last panel of FIG. 16, after reaction and washing, the presence and amounts of co-immobilized fluorescent tags are indicative of amounts of ligands that were present during the reaction.

In the embodiment of FIG. 16, the reporter may be a fluorescent tag, but it may also be an enzyme, a magnetic particle, or any number of detectible particles. Additionally, the RCANA could be immobilized on beads, but they could also be directly attached to a solid support via covalent bonds.

One advantage of this embodiment is that covalent immobilization of reporters allows stringent wash steps to be employed. This can be distinguished from to non covalent immobilization assays such as ELISA assays where stringent washing may destroy the signal. An additional advantage is that ribozyme ligases have the unique property of being able to transduce effectors into templates that can be amplified, affording an additional boost the in signal prior to detection.

Additionally, the method of the present invention contemplates that the RCANA construct may be amplified by polymerase chain reaction. Finally, the method contemplates that the RCANA oligonucleotide sequence of the construct may include RNA nucleotides, so that the method further includes reverse transcription of the RNA using reverse transcriptase to produce a DNA complementary to the RNA template.

Modified nucleotides may be introduced into the RCANA that substantially stabilize them from degradation in environments such as sera or urine. The analytical methods of the present invention do not rely on binding per se, but only on transient interactions. The present invention requires mere recognition rather that actual binding, thus providing a potential advantage of RCANA over antibodies. That is, even low affinities are sufficient for activation and subsequent detection, especially if individual immobilized RCANA are examined (i.e., by ligand-dependent immobilization of a quantum dot).

### F. In vitro Engineering and Selection of RCANAs for in vivo Applications

The above discussion has disclosed methods for the in vitro creation of RCANAs, and has disclosed some of their in vitro applications. In the following section we describe the design, engineering, and in vitro selection of RCANAs for in vivo applications.

This invention utilizes ribozymes that can alter the level of mRNAs in a cellular system. In one embodiment, the ribozyme can be a self splicing intron, such as the group I intron. This ribozyme can be inserted into a gene. If the ribozyme is active, it will catalyze the a self-splicing reaction that removes itself from the gene, allowing accurate expression of the gene. In another embodiment, the ribozyme may be one that acts in trans to cleave a mRNA. Again, changing the activity of the ribozyme will lead to a change in the level of the mRNA in the cell, thereby altering the level of the protein coded by that gene. Those skilled in the art will recognize that other ribozyme activities may be used. For the purpose of illustration, the invention is now described in detail with the use of the self splicing intron.

The intron is first modified to function as an RCANA. Briefly, the methods described above can be used generate RCANA introns. A pool of potential RCANA introns is created by randomizing one or more regions of the intron. The randomized region optionally includes one or more residues from the catalytic core. A selection protocol is then developed that allows the active RCANA introns to be partitioned from the inactive ones. For example, the active RCANA introns can be partitioned from the inactive RCANA intron based on the mobility in gel electrophoresis. Other methods will be clear to those skilled in the art. Based on this partitioning method, an iterative procedure of partitioning and subsequent amplification of the RCANA introns is used to select RCANAs that are regulated by an effector. With the exception of the partitioning method, this procedure is essentially identical to the selection described about for RCANA ligases.

As an alternative to the selection of RCANA introns, it is also possible to engineer RCANA introns. For example, one of the stem-loop structures of the intron can be replaced by an aptamer for the desired effector. Interaction of the effector-with this engineered RCANA intron-will result in a modulation of the RCANA intron activity. Because an aptamer is different from an regulatory element (as was detailed above), the 23 present method will, in general, lead to RCANAs that are regulated by the effector. however, as will be shown in an example below, an important aspect of the current invention is that this level of regulation can be adequate for in vivo applications.

### G. In vivo Selection and Optimization of RCANAs.

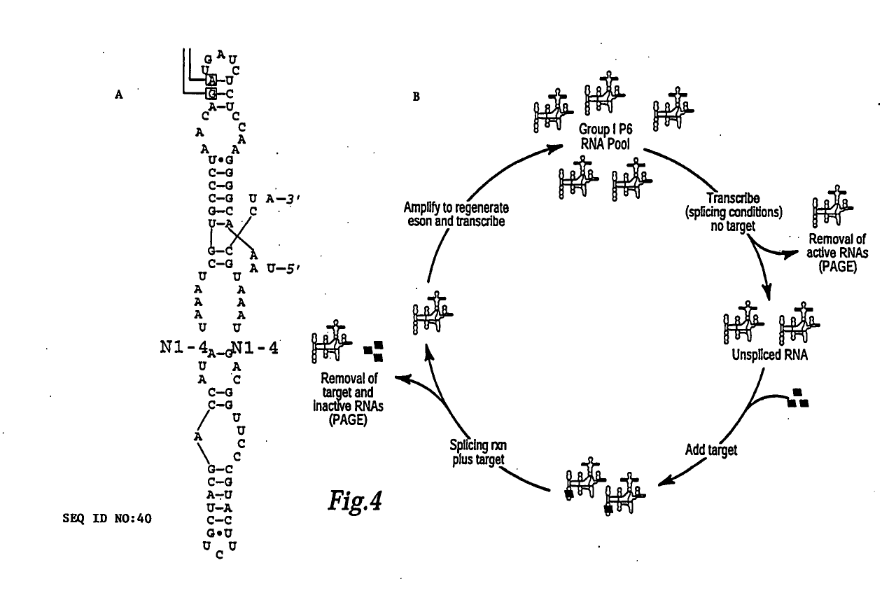

Here we disclose methods to generate RCANAs by using in vivo selection. FIG. 4b shows a selection protocol for the Group I P6 RCANA Pool of FIG. 4a. Positive and negative selections are made in vitro to select Group I RCANA that are dependent on activator. The selections are described above in Example 2 for a specific embodiment of the present invention—a theophylline dependent RCANA. In vivo screens and selections are used to select Group I RCANA that exhibit strong theophylline-dependence. The selected RCANA are mixed at various ratios with mutant Group I ribozymes that splice at a low but continuous level to determine the level at which RCANA can be selected against background. Because activation domains are often in the form of a stem-loop, the mutations can be concentrated in a single stem loop structure of the RCANA intron. In an alternate embodiment, the mutations can include catalytic residues. In yet another embodiment, the mutations are randomly dispersed in the intron. Finally, the best theophylline-dependent Group I aptazymes that have been derived by any of the methods described herein may undergo further selection by partially randomizing their sequences and selecting for improved in vivo performance.

Strategies similar to those depicted in FIGS. 4a and 4b may be used to develop RCANA on any desired effector. Positive and negative in vitro selection such as depicted in FIG. 4b are described above in Example 2 for a specific embodiment of the present invention.

From 10⁶ to 10¹⁰ variants can be efficiently transformed as described herein, sufficient to encompass most variants in the populations discussed so far. This efficiency of transformation, however, is likely to be insufficient to encompass a significant fraction of a completely random pool. Nonetheless, sequences have been selected from completely random expressed pools that can protect bacteria from bacteriophage infection.

The above procedure described how to select in vivo RCANAs. A similar procedure can be used to optimize engineered RCANAs. Residues in the RCANA that might include the ligand binding region, structural stem-loops, or even catalytic residues can be mutated. The selection procedure described above is then used to select for optimized RCANAs.

Since the rules that govern Group I intron splicing in different gene contexts are well known to those skilled in the art, the skilled artisan can remove RCANA introns from one context and modularly insert them into other genes. Should the efficiency or effector-dependence of intron splicing be compromised in the new gene, the intron may be reaccommodated to its new genetic environment by a selectable marker to the interrupted gene of interest and selecting for an effector-dependent phenotype.

Similarly, modulation of genes by cleavage is also well known to those skilled in the art. The skilled artisan can engineer endonucleolytic RCANAs that, upon activation, cause endonucleolytic cleavage of target nucleic acid. This endonucleolytic cleavage strategy may be applied to either upregulate or downregulate target polypeptide synthesis.

To the extent that aptazymes are self-sufficient, they should also function in eukaryotic cells, including human cells. Selecting for effector-dependence may also be performed in eukaryotic cells. Selection in eukaryotic systems may be performed, e.g., by fusing the gene of interest to a reporter gene such as GFP or luciferase. Variants of the RCANA that promote effector-dependent protein production may then be selected using a FACS. A pool of 10⁶ to 10⁸ variants may be screened by this procedure, a range comparable to the bacterial system previously described.

### H. In vivo Detection Applications

Using the present invention, it is possible to activate or repress a reporter gene (e.g., liciferase or GFP) containing an engineered RCANA in response to an endogenous protein activator, or a post-translationally modified form of an endogenous protein activator (e.g., protein kinases such as ERK 1 and phosphorylated ERK 1). It is also possible to activate or repress a reporter gene (e.g., luciferase or GFP) containing an engineered RCANA in response to small molecule effectors (e.g., cyclic AMP, glucose, bioactive peptides, bioactive nucleic acids, or low molecular weight drugs such as antibiotics, antineoplastics or the like.). Thus, reporter gene-engineered RCANA constructs may be used to monitor intracellular levels of proteins, post-translationally modified forms of proteins or small molecules such as cyclic AMP and the like. Such applications may be used for high-throughput cell-based assays and screens for drug leads or for drug optimization and development.

Bacterial strains such as Escherichia coli (E. coli), and Bacillils subtilis (B. subtilis), or yeast strains such as Saccharomyces cerevisiae (S. cerevisiae), and Schizosaccharomyces pombe (S. pombe) are transformed with an expression vector encoding a reporter gene regulated by a RCANA, and these engineered microbial cell lines are used for cell-based assays and tests for drug discovery and development. Similarly, standard mammalian cell lines such as CHO, NIH3T3, 293, and 293T are transfected with appropriate vectors (e.g., pCDNA, pCMV, or retrovirus), that are engineered to contain RCANA-regulatable reporter genes, and these re-engineered cell lines may be used subsequently for cell-based assays and tests. In another use of the RCANA reporter gene technology, tumorigenic cell lines such as LNCaP, MCF-7, IMAMB-435, SK-Mel, DLI, PC3, T47D and the like, may be transfected in vitro with appropriate vectors encoding an RCANA-regulatable reporter gene. These re-engineered tumorigenic cell lines may be used in cell-based screens for the discovery and development anti-neoplastic drugs.

In another in vivo application, reporter gene-RCANA constructs (e g., luciferase or GFP) may be used to generate live animal models for use in drug development. In one embodiment the RCANA construct may be used in an engineered tumorigenic cell line to indicate the levels of a target molecule used to generate a tumor xenograft in nude mice. Mice bearing the tumors derived from the engineered cell line may then be used to screen for drugs that alter the level of the target molecule. For example, a transfected MDA-MB-435 line engineered to express a GFP gene under regulatable control by intron response to the protein activator phospho-ERK 1 is used to screen for drugs which both inhibit tumor growth and block formation of phospho-ERK. In another embodiment of the RCANA intron invention, transgenic mouse models may be generated in which tissue or cell type specific expression of the reporter gene is controlled by the effector activated RCANA intron. For example transgenic mice expressing a phospho-VEGF receptor tyrosine kinase (RTK) specific RCANA regulated GFP gene under control of the MMTV (mouse mammary tumor virus) promoter would show expression of GFP in mouse mammary tissue in a phospho-VEGF RTK dependent manner. Furthermore, these mice may be used to screen compounds for anti-VEGF RTK activity.

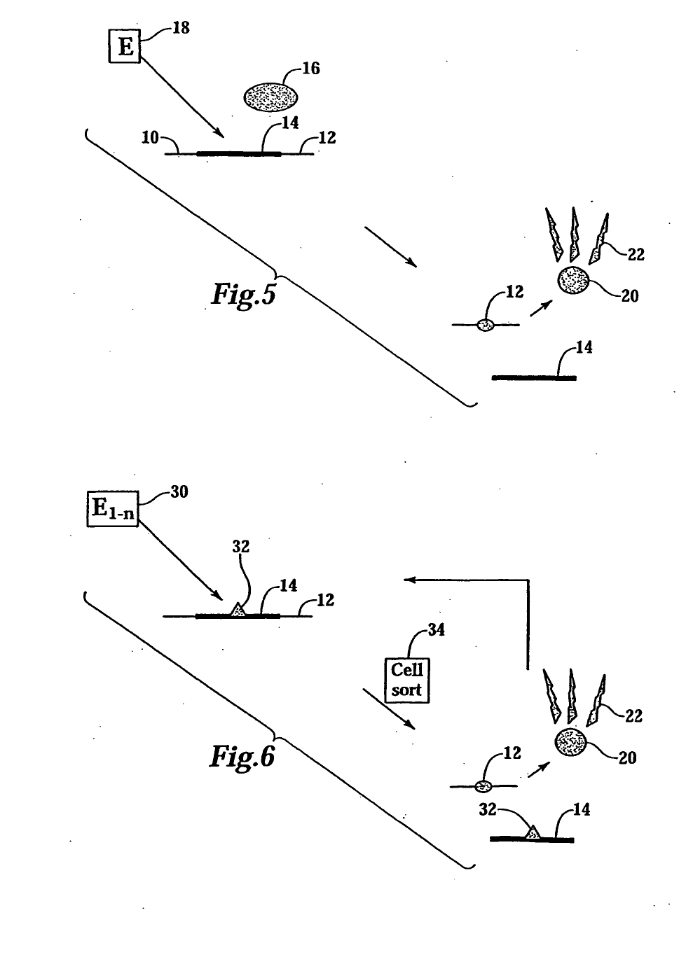

FIG. 5 is a diagrammatic representation of another embodiment of the present invention. Exogenous or endogenous activation of Group I intron splicing is depicted. A reporter gene such as Luciferase or beta-Gal is fused to the gene of interest which also contains the group I intron (td). Splicing-out of the Group I intron is induced by an effector, shown in the diagram as a protein, in this case Cyt18, by the shaded oval. Activation of the RCANA and auto-excision of the intron results in expression of the reporter gene to detect the desired reaction. The use of a reporter gene of this embodiment may be suitable for use in eukaryotic systems.

FIG. 6 is a diagram of another embodiment of the present invention. Libraries of candidate exogenous activators (E₁₋ₙ) may be generated from a randomized RCANA pool indicated by the triangle. As in the embodiment of FIG. 5, a reporter gene is expressed in cells where the exogenous activator activates the RCANA to release the intron from the gene. As will be known to those of skill in the art any number of current and future libraries may be used with the present invention.

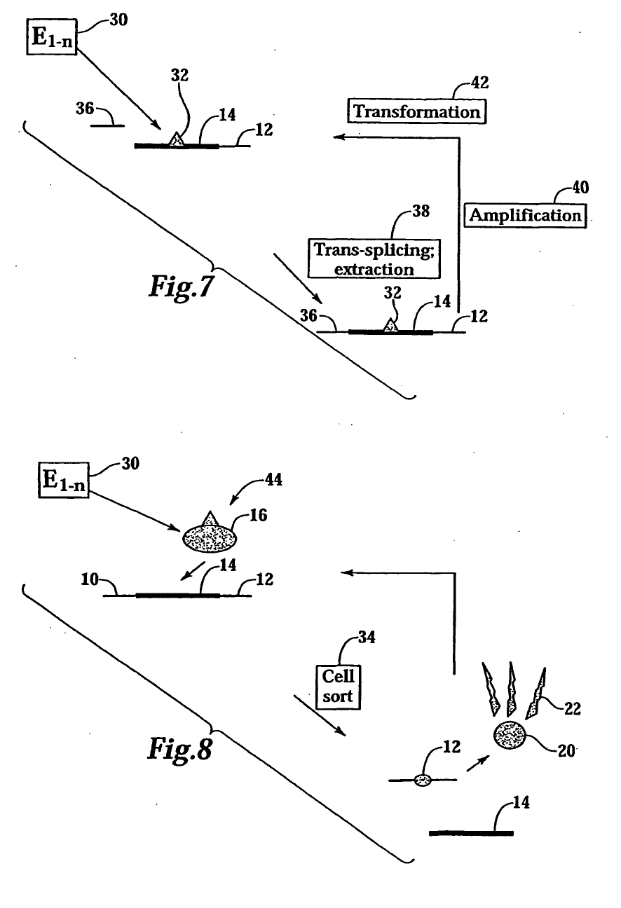

FIG. 7 depicts an alternative embodiment for screening libraries of exogenous activators. In the embodiment of the present invention of FIG. 7, Group I introns are induced into trans-splicing. Extracted and amplified introns are used to transform cells.

FIG. 8 shows yet another alternative embodiment for screening libraries of exogenous activators of the present invention. In the embodiment of FIG. 8, the effector (shaded oval), shown in this illustration as protein Cyt18, is mutagenized (triangle) to form an effector library. A second effector (E₁₋ₙ) interacts with and activates one or more members of the effector library. The effector-effector complex is exposed to the gene containing both the Group I intron and a reporter gene. Cell sorting reveals the cells that express the reporter gene to indicate successful activation of the RCANA by the effector-effector complex.

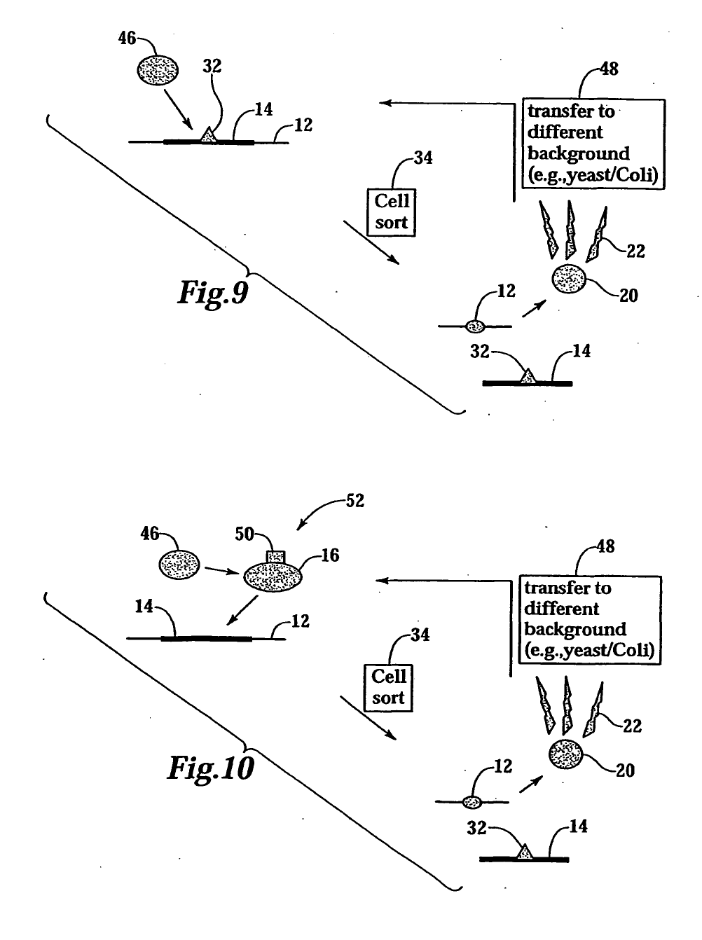

FIG. 9 is a diagram of an embodiment of the present invention for screening for endogenous activators. In this embodiment, an endogenous effector, in this illustration shown as a protein activator from endogenous or transformed origin (shaded oval), activates self-splicing of the Group I intron. Cell sorting is used to reveal the expression of the reporter gene. To protect against spontaneous auto-excision of the intron, the gene 27 may be transferred into a different background system such as yeast or E. Coli, for example.

FIG. 10 depicts an alternative to the embodiment of FIG. 9 to screen for endogenous activators of the present invention. In FIG. 10, the activator that is being screened for may include, inter alia, a phosphorylated protein, a product of ubiquitination, or a protein-protein complex. For example, a protein activator, shown as the small shaded oval, may phosphorylate an effector such as Cyt18, shown as a large shaded oval with the phosphorylation indicated by the shaded rectangle. The phosphorylated effector activates intron self-splicing with concomitant expression of the reporter gene, shown here for illustration as Luciferase or beta-Galactosidase.

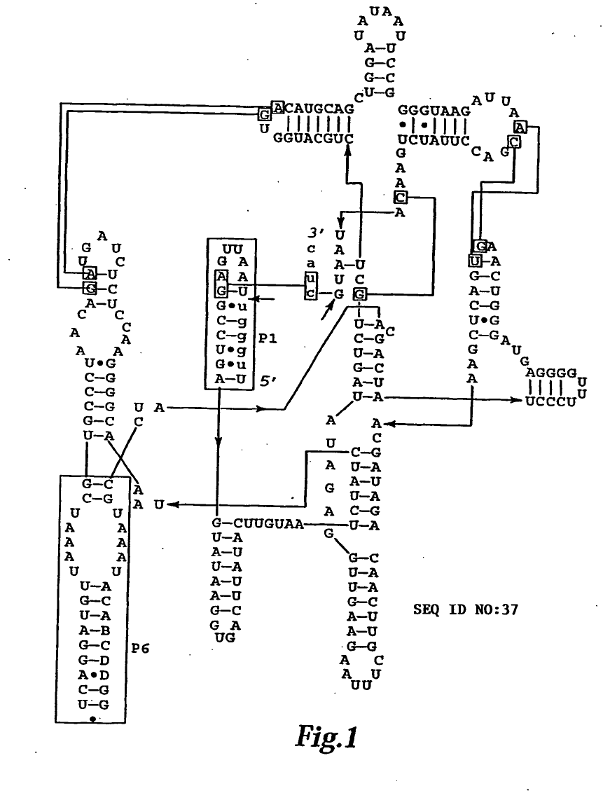

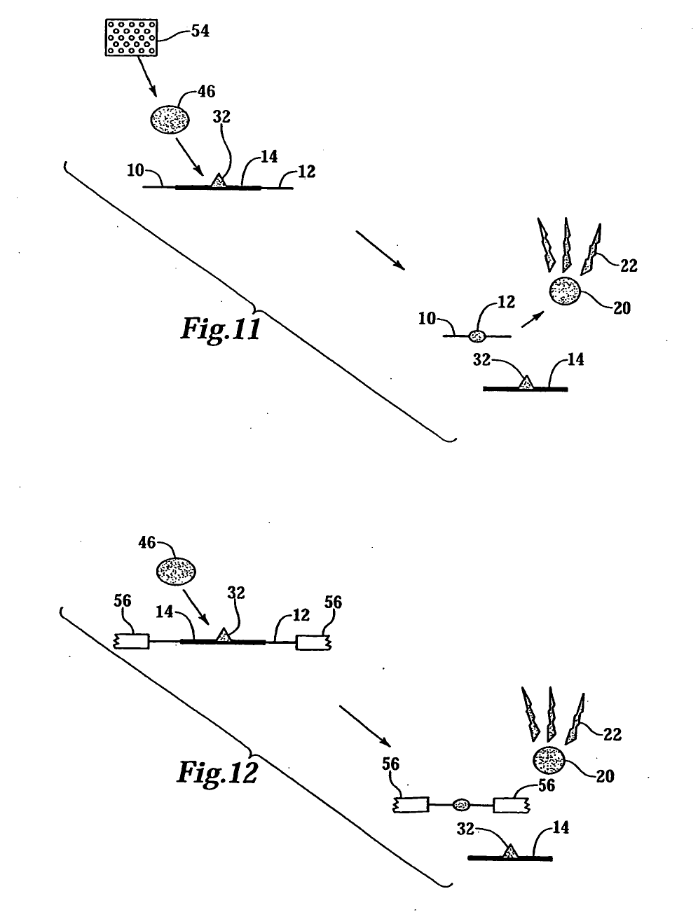

FIG. 11 shows yet another embodiment of the present invention to monitor compounds that perturb cellular metabolism. In this embodiment, a ribozyme similar to described in FIG. 6, and designated in this diagram by a line with a triangle is activated by a protein effector, shown as a shaded oval in FIG. 11. The protein effector may be a phosphoprotein, an induced protein, or a protein complex, for example. One or more second effectors, designated as a series of circles, alters the level of or degree of modification of the protein effector. The source of the second effectors may be endogenous or the effectors may be the product of a transformation construct used to transform a cell. Alteration of the level or modification of the protein effector results in an alteration in the expression of the reporter gene (shown as a dark circle with “lightning bolts”). The functioning of the gene of interest may thereby be perturbed, providing information about the function and/or regulation of the gene or gene product. FIG. 1 describes a method for taking the products of the screen described in FIGS. 8 and 10 and using them to monitor cellular or metabolic states.

FIG. 12 shows a further embodiment of the present invention that provides a non-invasive readout of metabolic states. An RCANA construct of the present invention may be introduced to a gene of interest. A protein suppressor from either an endogenous source from the product of cell transformation activates self-splicing of the RCANA, leading to expression of the endogenous gene, shown here again as a dark circle with lightning bolts. Whether or not the gene of interest is expressed upon release of the RCANA intron from the gene provides information about the metabolic state of the gene 28 of interest. The embodiment of the present invention of FIG. 12 thus provides a noninvasive means to determine the metabolic state of an organism with regard to a gene of interest.

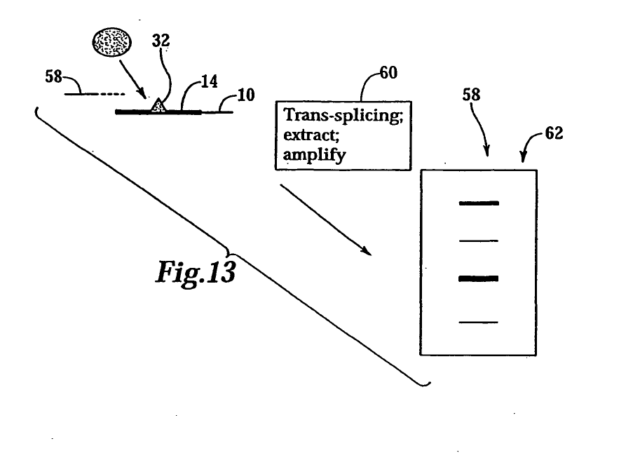

FIG. 13 depicts a further embodiment of the present invention wherein endogenous suppressors provide a non-invasive readout of multiple metabolic states. Multiple protein activators (endogenous or transformed) arc exposed to a pool of Group I introns of the present invention. The pool comprises introns with length polymorphisms that are depicted in FIG. 13 by a discontinuity or break in the line representing the Group I intron (thick line) residing in a gene of interest (thin line). Activation of the RCANA leads to trans-splicing among the various polymorphisms. The products of trans-splicing may be extracted and amplified. Separation of the trans-splicing products by gel clectrophoresis provides a read out of the protein function or the metabolic pathway. The readout may even be digitized for analysis.

In vivo Uses of RCANAs for Gene Therapy

One important feature of using RCANAs and the method of the present invention for gene therapies is that regulated introns may be used to control gene expression, for any of a variety of genes, since the introns may be inserted into and be engineered to accommodate virtually any gene. Moreover, since the RCANAs may be engineered to respond to any of a variety of effectors, the characteristics of the effector (oral availability, synthetic accessibility, pharmacokinetic properties) may be chosen in advance. The drug is chosen prior to engineering the target of the drug. In part because of these extraordinary capabilities, RCANA provide perhaps the only viable route to medically successful and practical gene therapies. Drugs may be given throughout the treatment (or lifetime) of a patient who had undergone a single initial gene therapy. In addition, by making the gene therapy regulatable, the amount of a gene product may be easily increased or decreased in different individuals at different times during the treatment by increasing or decreasing the doses of effectors.

The present method also includes transforming a cell with the RCANA construct so that the construct is inserted into a gene of interest. An effector is provided to activate the RCANA so that administering to the cell an effective amount of the effector induces the RCANA to splice itself out of the gene to regulate expression of the gene.

The method of the present invention contemplates that the RCANA construct may be a plasmid. The method then further includes transforming the cell with the plasmid. The method of the present invention also contemplates ligating the RCANA construct into a vector and transforming the cell with the vector.

### DEFlNITIONS

As used herein, the term “regulatable, catalytically active nucleic acid” or “RCANA” means a ribozyme or nucleic acid enzyme that is regulated by an effector.

As used herein, the term “regulatory domain” and “effector domain” are interchangeable terms meaning the region of effector binding on an RCANA.

The kinetic parameters of the RCANA may be varied in response to the amount of an effector, which may be an allosteric effector molecule. just as allosteric, protein enzymes undergo a change in their kinetic parameters or of their enzymatic activity in response to interactions with an effector molecule, the catalytic abilities of RCANAs may be similarly modulated by effectors. As demonstrated herein, the effectors may be small molecules, proteins, peptides or molecules that interact with proteins, peptides or other molecules. RCANAs transduce molecular recognition into catalysis upon interaction with an effector that interacts with a portion of the RCANA.

As used herein, the term “effector,” “effector molecule,” “allosteric effector” or “allosteric effector molecule” means a molecule or process that changes the kinetic parameters or catalytic activity of an RCANA.

As used herein, the term “catalytic residue” refers to residues that when mutated decrease the activity of the RCANA. Mutating a residue that affects the catalytic activity of a ribozyme following the selection of the RCANA, may cause different residues to become sensitive to mutation than in the original ribozyme. The relative mutational sensitivity of a given “catalytic residue” may change before and after the selection of the RCANA. These secondary mutations are also encompassed by the present invention.

As used herein, the term “aptamer” refers to a nucleic acid that has been specifically selected to optimally bind to a target ligand. As described above, it is important to recognize that an aptamer is fundamentally different than an RCANA.

As used herein, the term “kinetic parameters” refers to any aspect of the catalytic activity of the nucleic acid. Changes in the kinetic parameters of a catalytic RCANA produce changes in the catalytic activity of the RCANA such as a change in the rate of reaction or a change in substrate specificity. For example, self-splicing of an intron RCANA out of a gene environment may result from a change in the kinetic parameters of the RCANA. Similarly, cleavage of an endonucleolytic RCANA in a gene environment also may result fiom a change in the kinetic parameters of the RCANA.

As used herein, the term “catalytic” or “catalytic activity” refers to the ability of a substance to affect a change in itself or of a substrate under permissive conditions. As used herein, the term “protein-protein complex” or “protein complex” refers to an association of more than one protein. The proteins that make up a protein complex may be associated by functional, stereochemical, conformational, biochemical, or electrostatic mechanisms. It is intended that the term encompass associations of any number of proteins.

As used herein, the term “in vivo” refers to cellular systems and organisms, e.g., cultured cells, yeast, bacteria, plants and/or animals.

As used herein the terms “protein”, “polypeptide” or “peptide” refer to compounds comprising amino acids joined via peptide bonds and are used interchangeably. As used herein, the term “endogenous” refers to a substance the source of which is from within a cell, cell extract or reaction system. Endogenous substances are produced by the metabolic activity of, e.g., a cell. Endogenous substances, however, may nevertheless be produced as a result of manipulation of cellular metabolism to, for example, make the cell express the gene encoding the substance.

As used herein, the term “exogenous” refers to a substance the source of which is external to a cell, cell extract, or reaction system. An exogenous substance may nevertheless be internalized by a cell by any one of a variety of metabolic or induced means known to those skilled in the art.

As used herein the term “modified base” refers to a non-natural nucleotide of any sort, in which a chemical modification may be found on the nucleobase, the sugar, or the polynucleotide backbone or phosphodiester linkage.

As used herein, the term “gene” means the coding region of a deoxyribonucleotide sequence encoding the amino acid sequence of a protein. The term includes sequences located adjacent to the coding region on both the 5′and 3′ ends such that the deoxyribonucleotide sequence corresponds to the length of the full-length mRNA for the protein. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “3′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

The term “gene of interest” as used herein refers to a gene, the function and/or expression of which is desired to be investigated, or the expression of which is desired to be regulated, by the present invention. In the present disclosure, the td gene of the T4 bacteriophage is an example of a gene of interest and is described herein to illustrate the invention. The present invention may be useful in regard to any gene of any organism, whether of a prokaryotic or eukaryotic organism.

The term “hybridize” as used herein, refers to any process by which a strand of nucleic acid binds with a complementary strand through base pairing. Hybridization and the strength of hybridization (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids.

The terms “complementary” or “complementarity” as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology,” as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence. When used in reference to a single-stranded nucleic acid sequence, the term “substantially homologous” refers to any probe which can hybridize (i.e., it is the complement to the single-stranded nucleic acid sequence under conditions of low stringency as described).

As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g, the presence or absence of formamide, dextran sulfate and/or polyethylene glycol) are considered and the hybridization solution may be varied to generate conditions of either low or high stringency different from, but equivalent to, the above listed conditions.

As used herein the term “stringency” is used in reference to the conditions of temperature, ionic strength, and the presence of other compounds such as organic solvents, under which nucleic acid selections are conducted. With “high stringency” conditions a relatively small number of nucleic acid catalysts will be selected from a random sequence pool, while under “low stringency conditions” a larger number of nucleic acid catalysts will be selected from a random sequence pool.

Numerous equivalent conditions may be employed to comprise low or high stringency conditions; factors such as the length of incubation of the reaction, the presence of competitive inhibitors of the reaction, the buffer conditions under which the reaction is carried out, the temperature at which the reaction is carried out are considered and the hybridization solution may be varied to generate conditions of low stringency selection different from, but equivalent to, the above listed conditions.

The term “antisense,” as used herein, refers to nucleotide sequences that are complementary to a specific DNA or RNA sequence. The term “antisense strand” is used in reference to a nucleic acid strand that is complementary to tile “sense” strand. Antisense molecules may be produced by any method, including synthesis by ligating the gene(s) of interest in a reverse orientation to a viral promoter that permits the synthesis of a complementary strand. Once introduced into a cell, the transcribed strand combines with natural sequences produced by the cell to form duplexes. These duplexes then block either the further transcription or translation. In this manner, mutant phenotypes may also be generated. The designation “negative” is sometimes used in reference to the antisense strand, and “positive” is sometimes used in reference to the sense strand. The term is also used in reference to RNA sequences that are complementary to a specific RNA sequence (e g., mRNA). Included within this definition are antisense RNA (“asRNA”) molecules involved in genetic regulation by bacteria.

Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used. in reference to a nucleic acid strand that is complementary to the “sense” strand. The designation. (i.e., “negative”) is sometimes used in reference to the antisense strand with the designation (+) sometimes used in reference to the sense (i.e., “positive”) strand.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

“Transformation,” as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. The term “transfection” as used herein refers to the introduction of foreign DNA into eukaryotic cells.

Transfection may be accomplished by a variety of methods known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell that has stably integrated foreign DNA into the genomic DNA. The term also encompasses cells that transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J. et al., Molecular Cloning: A Laboratory Manual, 2nd ed., Cold Spring Harbor Laboratory Press, New York (1989),pp. 16.9-16.15.

As used herein, the term “reporter gene” refers to a gene that is expressed in a cell upon satisfaction of one or more contingencies and which, upon expression, confers a detectable phenotype to the cell to indicate that the contingencies for expression have been satisfied. For example, the gene for Luciferase confers a luminescent phenotype to a cell when the gene is expressed inside the cell. In the present invention, the gene for Luciferase may be used as a reporter gene such that the gene is only expressed upon the splicing out of an intron in response to an effector. Those cells in which the effector activates splicing of the intron will express Luciferase and will glow.

As used herein, the term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify”, when used in reference to nucleic acids refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact length of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence. As used herein, the term “polymerase chain reaction” (“PCR”) refers to the method of K.B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence.

To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be PCR amplified”.

With PCR, it is possible to amplify a single copy of a specific target sequence in genomic DNA to a level detectable by several different methodologies, e.g., hybridization with a labeled probe; incorporation of biotinylated primers followed by avidin-enzyme conjugate detection; incorporation of ³² P-labeled deoxynucleotide triphosphates, such as DCTP or DATP, into the amplified segment. In addition to genomic DNA, any oligonucleotide sequence can be amplified with the appropriate set of primer molecules. In particular the amplified segments created by the PCR process itself are, themselves, efficient templates for subsequent PCR amplifications.

As used herein, the term “metabolic pathway” means a physical or chemical process found in a living organism by which a substance is produced and maintained, e.g., anabolism, and also the transformation by which energy is made available for the use of a living organism, e.g., catabolism.

As used herein, the term “bioproduct” means a substance produced by a physical or chemical process, the components of which are found in a living organism(s).

As used herein, the term “biosynthetic process” means a process by which a substance is produced using the physiological process found in a living organism.

### EXAMPLE 1: GPITH1P6

### Engineering of an RCANA for In vivo Detection Applications

The first example illustrates how to make an RCANA construct and demonstrates self-splicing of the RCANA out of a gene in response to an effector molecule.

Construction of a RCANA.

| | |
|---|---|
| Oligos GplWt3.129: | |
| 5-TAA TCT TAC CCC GGA ATT | (SEQ ID NO:1) |
| ATA TCC AGC TGC ATG TCA CCA TGC AGA GCA GAC TAT ATC TCC AAC TTG | |
| TTA AAG CAA GTT GTC TAT CGT TTC GAG TCA CTT GAC CCT ACT CCC CAA | |
| AGG GAT AGT CGT TAG-3′ and | |
| GpITh1P6.131: | |
| 5-GCC TGA GTA TAA GGT GAC TTA TAC TTG TAA TCT | (SEQ ID NO:2) |
| ATC TAA ACG GGG AAC CTC TCT AGT AGA CAA TCC CGT GCT AAA TTA TAC | |
| CAG CAT CGT CTT GAT GCC CTT GGC AGA TAA ATG CCT AAC GAC TAT CCC | |
| TT-3′ | |

were annealed and extended in a 30 μl reaction containing 100 pmoles of each oligo, 250 mM Tris-HCl (pH 8.3), 40 mM MgCl₂, 250 mM NaCl, 5 mM DTT, 0.2 mM each dNTP, 45 units of AMV reverse transcriptase (RT: Amersham Pharmacia Biotech, Inc., Piscataway, N.J.) at 37° C. for 30 min. The extension reaction was diluted 1 to 50 in H₂O.

A PCR reaction containing 1 μl of the extension dilution, 500 mM KCl, 100 mM Tris-HCl, (pH 9.0), 1% Triton® X-100, 15 mM MgCl₂, 0.4 μM of GpIWt1.75: 5′-GAT AAT ACG ACT CAC TAT AGG GAT CAA CGC TCA GTA GAT GTT TTC TTG GGT TAA

TTG AGG CCT GAG TAT AAG GTG-3′ (SEQ ID NO:3)),0.4 μM of Gp1Wt4.89:5′-CTT AGC TAC AAT ATG AAC TAA CGT AGC ATA TGA CGC AAT ATT AAA CGG TAG CAT TAT GTT CAG ATA AGG TCG TTA ATC TTA CCC CGG AA-3′ (SEQ ID NO:4), 0.2 mM each dNTP and 1.5 units of Taq polymerase (Promega, Madison, Wis.) was thermocycled 20 times under the following regime: 94° C. for 30 sec, 45° C. for 30 sec, 72° C. for 1 min. The PCR reaction was precipitated in the presence of 0.2 M NaCl and 2.5 volumes of ethanol and then quantitated by comparison with a molecular weight standard using agarose gel electrophoresis.

The RCANA construct was transcribed in a 10 μl high yield transcription reaction (AmpliScribe from Epicentre, Madison, Wis. The reaction contained 500 ng PCR product, 3.3 pmoles of ³²P [³²P]UTP, 1×AmpliScribe transcription buffer, 10 mM DTT, 7.5 mM each NTP, and 1 μl AmpliScribe T7 polymerase mix. The transcription reaction was incubated at 37° C. for 2 h. One unit of RNase free-DNase was added and the reaction returned to 37° C. for 30 min. The transcription was then purified on a 6% denaturing polyacrylamide gel to separate the full length RNA from incomplete transcripts and spliced products, eluted and quantitated spectrophotometrically.

In vitro Assay. The RNA (4 pmoles/12 μl H₂O) was heated to 94° C. for 1 min then cooled to 37° C. over 2 min in a thermocycler. The RNA was divided into 2 splicing reactions (9 μl each) containing 100 mM Tris-HCl (pH 7.45), 500 mM KCl and 15 mM MgCl₂, Plus or minus theophylline (2 mM). The reactions were immediately placed on ice for 30 min. GTP (1 mM) was added to the reactions (final volume of 10 μl) and the reactions were incubated at 37° C. for 2 h.

The reactions were terminated by the addition of stop dye (10 μl) (95% formamide, 20 mM EDTA, 0.5% xylene cyanol, and 0.5% bromophenol blue). The reactions were heated to 70° C. for 3 min and 10 μl was electrophoresed on a 6% denaturing polyacrylamide gel. The gel was dried, exposed to a phosphor screen and analyzed using a Molecular Dynamics Phosphorimager (Sunnyvale, Calif.).

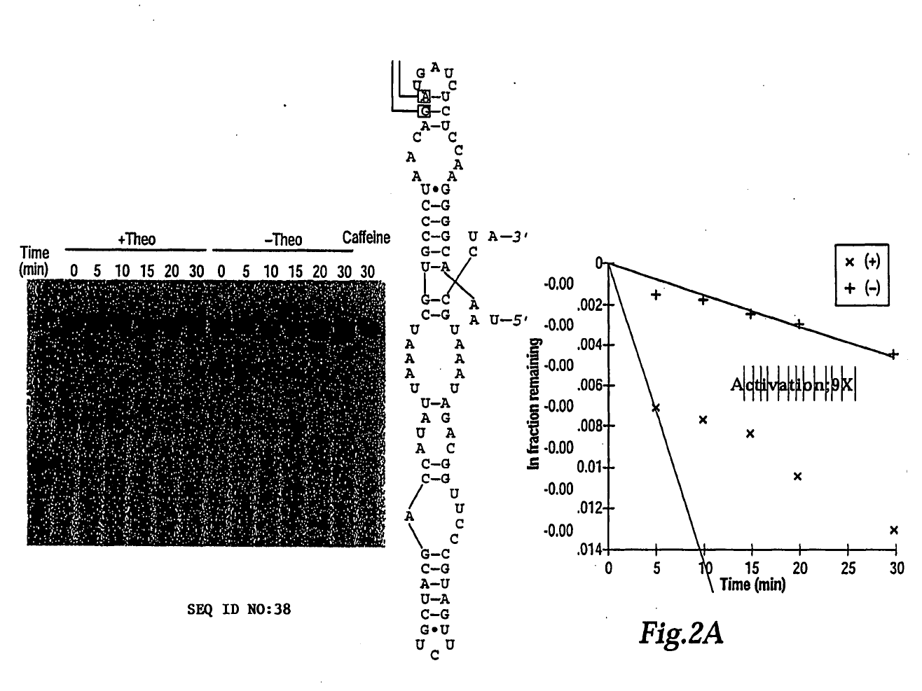

Activation was determined from the amount of circular intron in each reaction. Circularized introns migrate slower than linear RNA and can be seen as the bands above the dark bands of linear RNA in the +Theo lanes of the gels of FIGS. 2a and 2b.

In vivo Screening of Group I Aptazymes. The RCANA constructs as well as the wild type and a negative control were ligated into a vector that contains the T4 td intron with Eco R I and Spe I flanking the P6 region, transformed and miniprepped. The plasmids were then transformed into C600:Thy A Kan^{R} cells (cells lacking thymidine synthetase).

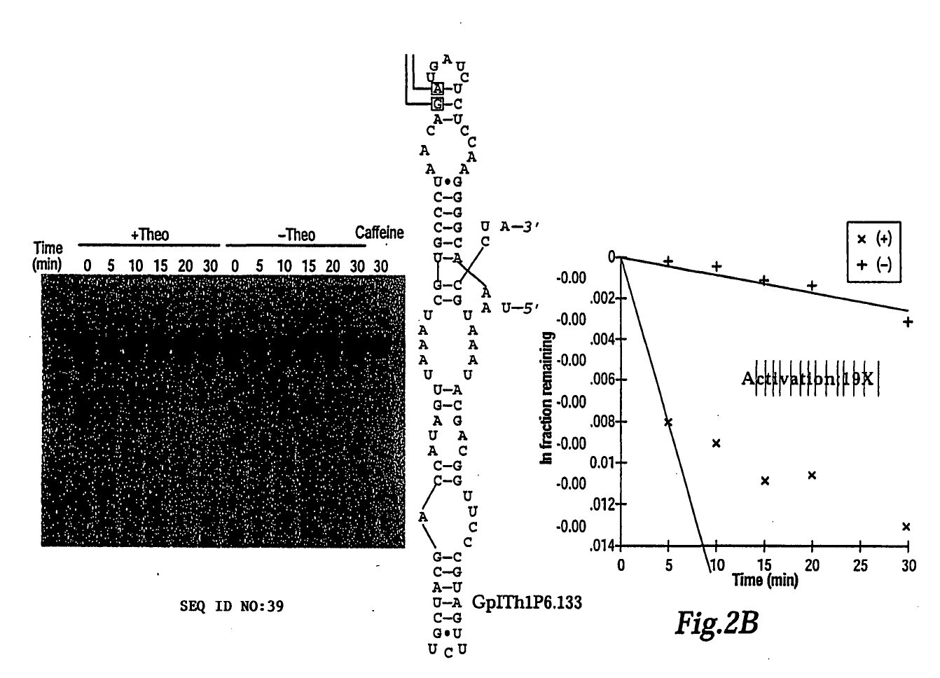

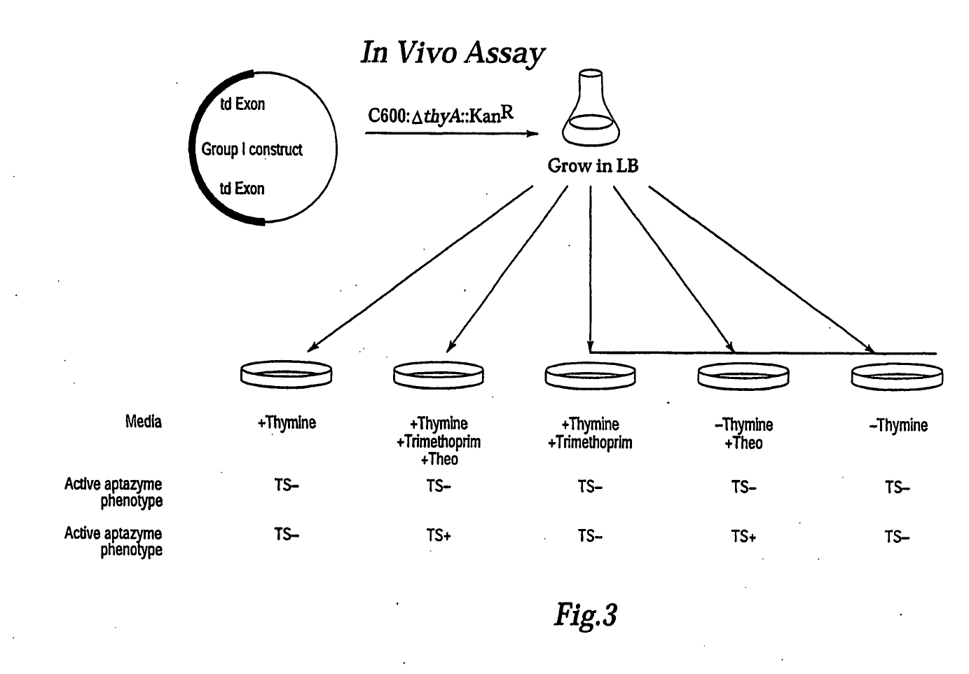

Individual colonies were picked and grown in rich media overnight. Theophylline (1 μl: 6.6 mM) or H₂O (1 μl) was added to 2 μl of the overnight growth and was spotted on either minimal media plates, or minimal media plates with thymine. (See FIG. 3)

### EXAMPLE 2: GPIP6THPOOL

### In vitro Selection to Optimize an RCANA for In vivo Detection Applications

Example 2 illustrates how to generate a population of RCANA so that there is variation in the nucleotide sequence of the aptamers. This example also illustrates how to select for phenotypes that are responsive to an effector molecule from among that population of RCANA.

Construction of the Pool. The construction of the pool was similar to the construction of the individual engineered RCANA constructs. Oligos Gp1Wt3.129 and GpIThP6pool:

5′-GCC TGA GTA TAA GGT GAC TTA TAC TAG TAA TCT ATC TAA ACG GGG AAC CTC TCT AGT AGA CAA TCC CGT GCT AAA TN(1-4)A TAC CAG CAT CGT CTT GAT GCC CTT GGC AGN(1-4) TAA ATG CCT AAC GAC TAT CCC TT-3′ (SEQ ID NO:5) were extended in the same manner as above. The extension reaction was diluted and used as template for a PCR reaction. The PCR reaction was similar to the reaction described with the following exceptions: the volume was doubled and GpIWt4.89 was replaced with Gp 1MutG. 101: 5′-CTT AGC TAC AAT ATG AAC TAA CGT AGC ATA TGA CGC AAT ATT AAA CGG TAG TAT TAT GTT CAG ATA AGG TCG TTA ATC TTA CCC CGG AAT TCT ATC CAG CT-3′ (SEQ TD NO:6) in which there is an G to A mutation at the terminal residue of the intron. The pool had a diversity of 1.16×10⁵ molecules. RNA was made as described above.

In vitro Negative Selection. The RNA (10 pmoles/70 μl H₂O) was heated to 94° C. for 1 min then cooled to 37° C. over 2 min in a thermocycler. The splicing reaction (90 μl) contained 100 mM Tris-HCl (pH 7.45), 500 mM KCl and 15 mM MgCl₂. The reaction was immediately placed on ice for 30 min. GTP (1 mM) was added to the reaction (final volume of 100 μl) and the reaction was incubated at 37° C. for 20 h. The reaction was terminated by the addition 20 mM EDTA and precipitated in the presence of 0.2 M NaCl and 2.5 volumes of ethanol. The reaction was resuspended in 10 ml H₂O and 10 μl stop dye and heated to 100° C. for 3 min and was electrophoresed on a 6% denaturing polyacrylamide gel with Century™ Marker ladder (Ambion, Austin, Tex.). The gel was exposed to a phosphor screen and analyzed. The unreacted RNA was isolated from the gel, precipitated and resuspended in 10 μl of H₂O.

Positive Selection. The RNA (5 μl of negative selection) was heated to 94° C. for 1 min then cooled to 37° C. over 2 min in a thermocycler. The positive splicing reaction (45 μl) contained 100 mM Tris-HCl (pH 7.45), 500 mM KCl, 15 mM MgCl₂ and 1 mM theophylline. The reaction was immediately placed on ice for 30 min. GTP (1 mM) was added to the reaction (final volume of 50 μl) and the reaction was incubated at 37° C. for 1 h. The reaction was terminated by the addition of stop dye, heated to 70° C. for 3 min and was electrophoresed on a 6% denaturing polyacrylamide gel with Century™ Marker ladder. The gel was exposed to a phosphor screen and analyzed. The band corresponding to the linear intron was isolated from the gel and precipitated and resuspended in 20 μl H₂O-Amplification and Transcription. The RNA was reverse transcribed in a reaction containing 250 mM Tris-HCl (pH 8.3), 375 mM KCl, 15 mM MgCl₂, 1 M DTT, 0.4 mM of each dNTP 2 pM GpIMutG.101 and 400 units of SuperScript II reverse transcriptase (Gibco BRL, Rockville, NW). The cDNA was then PCR amplified, transcribed and gel purified as described above.

FIG. 3 depicts an in vivo assay system for Group I introns of the present invention. The td intron normally sits within the td gene for thymidylate synthase (TS) in phage T4. A ThyA E. coli host that lacks cellular TS is unable to grow in the absence of exogenous thymine or thymidine (-Thy). The cloned td gene can complement the ThyA cells and grow on -Thy media. Conversely, cells that lack TS have a selective advantage on media containing thymidine and trimethoprim. Therefore, cells harboring theophylline-responsive Group I aptazymes grow better in the presence of theophylline and the absence of thymidine. In contrast, the same cells grow better in the absence of theophylline and the presence of thymidine and trimethoprim.

This strategy provides both a positive in vivo screen and selection for theophylline-dependent activation and a negative in vivo screen and selection for theophylline-absent repression. The assay system of FIG. 3 was used in Example 1, above, for the in vivo screening of Group I aptazymes in a specific embodiment of the present invention.

FIG. 4a depicts the critical residues of the P6 region of the Group I ribozyme joined to the anti-theophylline aptamer by a short randomized region to generate a pool of RCANA of the present invention. The residues shown in bold in FIG. 4a are the P6 critical residues, and the faded residues shown in FIG. 4a are the anti-theophylline aptamer. The randomized regions are designated in FIG. 4a as “N 1-4”. Approximately 40 random sequence residues are introduced into the NI-4 region of the construct to synthesize a pool of RCANA, referred to herein as a communication module pool.

### EXAMPLE 3: POLYPEPTIDE DEPENDENT REGULATABLE, CATALYTICALLY ACTIVE NUCLEIC ACIDS

Natural nucleic acids frequently rely on proteins for stabilization or catalytic activity. In contrast, nucleic acids selected in vitro can catalyze a wide range of reactions even in the absence of proteins. In order to augment selected nucleic acids with protein functionalities, the present invention includes a technique for the selection of protein dependent ribozyme ligases.

The catalytic domain of the ribozyme ligase, L1, was randomized, and variants that required one of two protein cofactors, a tyrosyl tRNA synthetase (Cyt18) or hen egg white lysozyme, were selected. The resultant regulatable, catalytically active nucleic acids were activated thousands of fold by their cognate, protein effectors, and could specifically recognize the structures of the native proteins. Protein-dependent regulatable, catalytically active nucleic acids are adaptable to novel assays for the detection of target proteins, and the generality of the selection method, as demonstrated herein allows for the identification of regulatable, catalytically active nucleic acids using high-throughput methods and equipment. These regulatable, catalytically active nucleic acids are able to, for example, recognize a sizable fraction of a proteome.

It has been recognized that it is possible to design and select effector-modulated ribozymes (RCANA) that show astounding activation parameters relative to allosteric proteins. For example, Breaker and his co-workers engineered an allosteric hammerhead ribozyme that is inhibited by 180-fold in the presence of a small molecule, ATP (Tang, J. & Breaker, R. R. Rational design of allosteric ribozymes. Chem. Biol. 4, 453-459 (1997)). An effector-activated ribozyme ligase that is activated by 1,600-fold in the presence of theophylline (Robertson, M. P. & Ellington, A. D., Design and optimization of effector activated ribozyme ligases. Nucleic Acids Res. 28, 1751-1759 (2000)) has also been engineered. Allosteric domains have also been selected from random sequence pools appended to the hammerhead ribozyme; these domains mediate a 5,000-fold activation of the ribozyme by other small molecules, e.g., cyclic nucleotide monophosphates (Koizumi, M., Soukup, G. A., Kerr, J. N. & Breaker, R. R., Allosteric selection of ribozymes that respond to the second messengers cGMP and cAMP. Nat. Struct. Biol. 6, 1062-1071 (1999)).

The present inventors recognized and herein demonstrate that it is possible to identify not only ribozymes, but nucleic acid segments that are activated by protein effectors. They further recognized that previous attempts to isolate ribozymes had required active catalytic domains within those ribozymes. All previously isolated ribozymes had been designed, modified, isolated or identified with natural or enhanced catalytic domains, hence the isolation of these ribozymes are extremely dependent on the catalytic domain for their isolation.

The RNAse P ribozyme from eubacteria has been shown to catalyze the cleavage of tRNA, it is normally complexed with a protein (P-protein) that substantially enhances its activity. Similarly, the Group I intron NDI is extremely dependent on Cyt18, a tyrosyl tRNA synthetase from Neurospora crassa mitochondria, while the tertiary structure of the intron bI5 is stabilized by its cognate protein, CBP2. Proteins have been frequently found to assist in the folding of RNA molecules, acting as chaperons to partially solvate the polyanionic backbone (Weeks, K. M. Protein-facilitated RNA folding. Curr. Opin. Struct. Biol. 7,336-342 (1997)).

The present invention includes a generalized selection scheme for the isolation of regulatable, catalytically active nucleic acids. Using the present invention a novel class of not just ribozymes, but rather, regulatable, catalytically active nucleic acids that are specifically activated thousands of fold by protein effectors such as Cyt18 and lysozyme have been create isolated and identified.

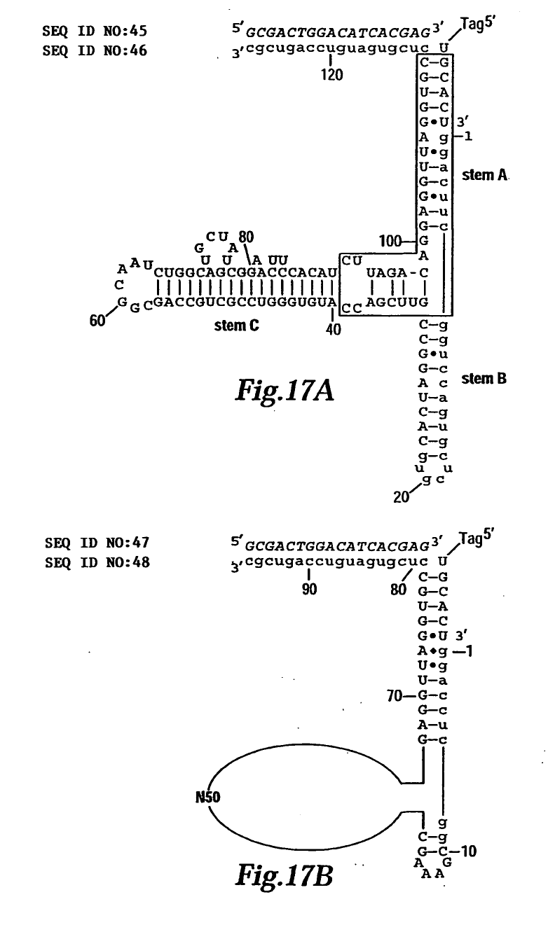

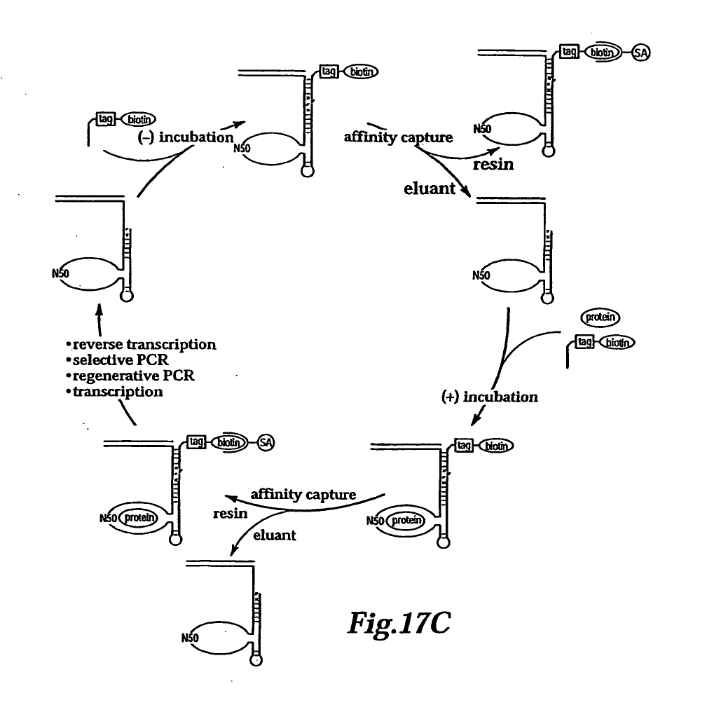

In vitro selection of protein-dependent ribozymes. While attempting to identify peptide- and protein-dependent ribozymes the present inventors used novel strategies for the design and selection of ribozymes that were activated by small molecular effectors. However, when peptide-and protein-binding sites were appended to stem C of the small L1 ligase (FIG. 17A) little or no modulation of activity was observed in the presence of cognate peptide or protein effectors. Similarly, when a random sequence loop was introduced at the termini of stem C, selection for protein-dependent variants produced only very modest activation (<2×).

It was then discovered that engineering protein-dependent ribozymes required fundamentally different principles than engineering small molecule-dependent ribozymes. In particular, it was recognized that small molecules that bind to limited allosteric sites in turn to potentiate small but significant reorganizations of the secondary and tertiary structures of core ribozymes. It was further discovered that larger effector molecules such as proteins, bind to much larger sites and might sterically inhibit the catalytic core. Therefore, it was necessary to include the catalytic core in the selection. To this end, a nucleic acid segment pool based on the L1 ligase (L1-N50) in which critical catalytic residues were also randomized (FIG. 17B) was designed.

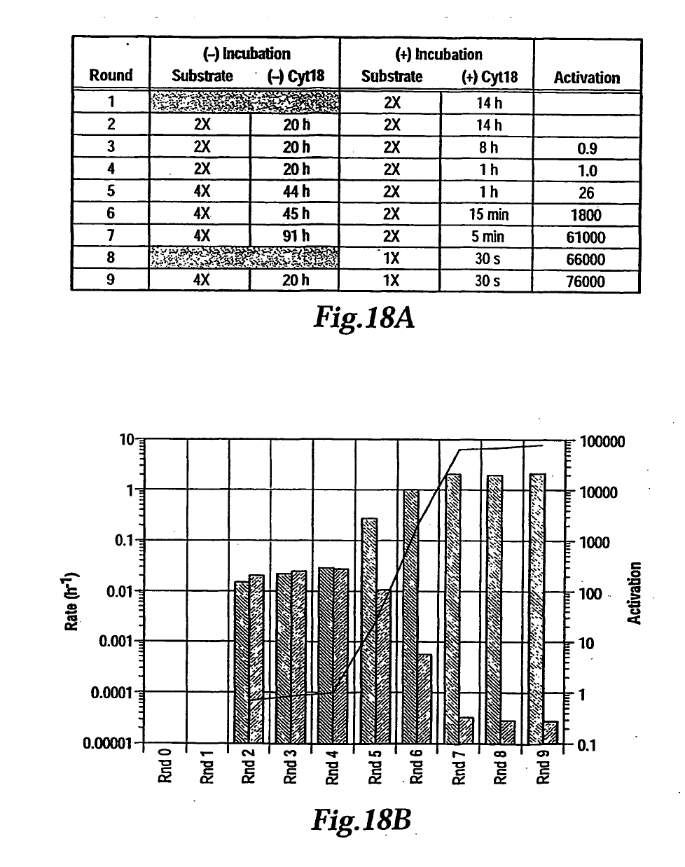

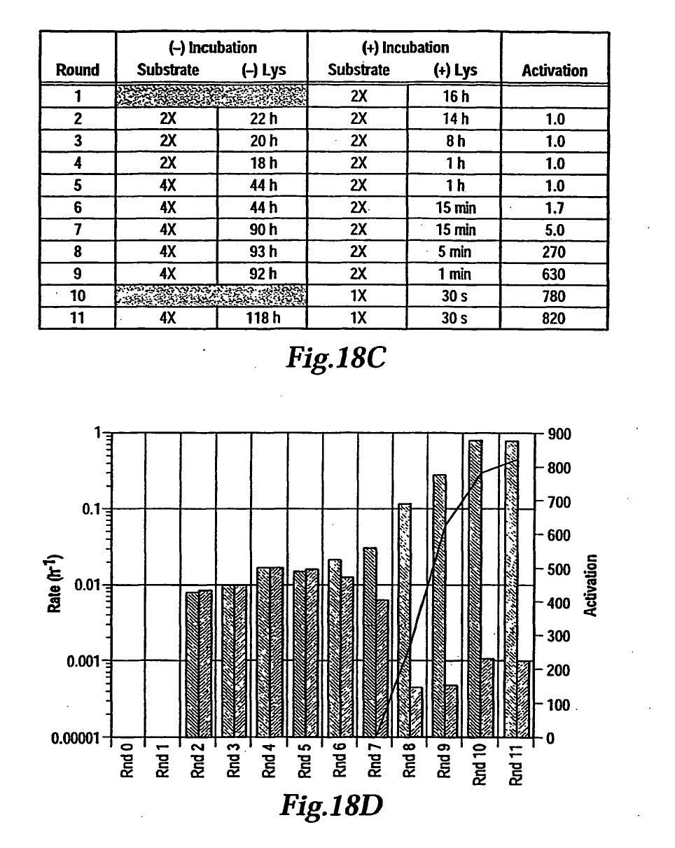

The L1-N50 pool (10¹⁵ starting species) was subjected to an iterative regime of negative and positive selections for ligation activity (FIG. 17C). The pool was initially incubated with a biotinylated substrate and reactive species were removed; the pool was then mixed with the effector molecule, a tyrosyl tRNA synthetase from Neurospora mitochondria (Cyt18), and reactive species were removed and amplified. The Cyt18 protein was chosen as an effector because it was known to both tightly bind (K_{d} in the femptomolar range) and activate a natural RNA catalyst, a group I self-splicing intron. During the course of these studies, and in negative selection screens in general using the present invention, the stringency of the negative selections may be increased by increasing the time allowed for ligation and substrate concentration in the absence of Cyt18. Conversely, the stringency of the positive selections may increased by decreasing the time allowed for ligation and the substrate concentration (FIG. 18A).

The degree of protein-dependent activation was assessed in a standard assay, and progressively increased from Round 5 onwards (FIG. 18B). By Round 7, protein-dependent activation was greater than 50,000-fold. At the conclusion of the selection it had risen to over 75,000-fold. The most prevalent clone in the selected population (cyt72) performed the ligation reaction with an observed rate of 1.6 h⁻¹ in the presence of Cyt18, but this rate dropped to 0.00005 h⁻¹ when the protein was left out of the reaction, a difference of 34,000-fold. Another clone (cyt9-18) from the selection had even better activation parameters, ligating at a rate of 2.1 h⁻¹ with Cyt18 included in the reaction, but only 0.00002 h⁻¹ without protein for a difference of 94,000-fold. Importantly, these values are many orders of magnitude greater than the known ligand-mediated activation of allosteric protein enzymes, and are 10- to 100-fold greater than the previously observed activation of ribozymes by small molecule effectors.

While the extent of Cyt18 activation of the aptazyme ligase was impressive, Cyt18 had previously been shown to similarly activate a group I self-splicing intron. In order to determine whether the ability to select for protein-dependent activation of ribozyme catalysis was specific to certain types of proteins or was a more general phenomena, ribozyme ligases that could be activated by a protein not normally known to bind RNA, hen egg white lysozyme were isolated. Using the same selection scheme and progressive increases in stringency (FIG. 18C), regulatable, catalytically active nucleic acids that were activated by lysozyme were isolated in 11 cycles of selection and amplification. The final, selected population was activated about 800-fold by lysozyme (FIG. 18D) and an isolated clone, lys 11-2, exhibited a 3100-fold activation, ligating with an observed rate of 0.6 h⁻¹ in the presence of lysozyme but only 0.0002 h⁻¹ without lysozyme.

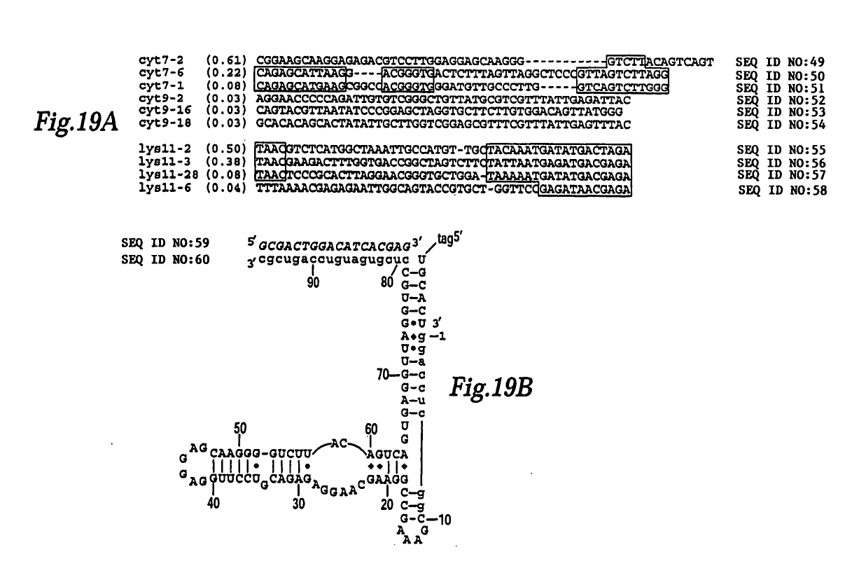

Characterization of protein-dependent ribozymes. Individual ribozymes were cloned from both selections and sequenced (FIG. 19A). In both instances, only a few families of ribozymes remained. These results are more in line with those previously observed for ribozyme selections with small organic ligands. Using the present invention, individual sequences could be folded to fit within the general structural context of the L1 ligase (FIG. 19B). The selected ribozymes were still highly dependent on the presence of the 3′ primer for activity, as was the parental L1 ligase. The selected sequences were hypothesized to form extended ‘stem C’ structures. The formation of such extended stems was again consistent with L1 ligase.

The distal portion of stem C, adjacent to the hairpin, was not conserved following partial randomization and re-selection, indicating that this portion of the ribozyme was not critical for activity. Moreover, the distal, hairpin portion of stem C can be shortened without loss of activity, and the hairpin may be replaced by aptamers that bind small organic ligands to generate regulatable, catalytically active nucleic acids. While the internal loop region of stem C, adjacent to the 3-arm junction, was conserved following doped sequence selection, complete randomization of this region followed by selection for ligase function yielded a variety of sequence solutions. Therefore, the selected protein-dependent ribozymes differed substantially from the parental ribozyme in this region.

Specificity of activation. In order to assess the specificity of activation of selected ribozymes by protein effectors, the Cyt 18-dependent population was incubated with a variety of proteins, including lysozyme, E. coli tryptophanyl tRNA synthetase, ricin A chain, and MS2 coat protein. No activation was observed with proteins that were not used during the isolation. Similarly, lysozyme-dependent clones were incubated with Cyt18, turkey lysozyme, and lysozyme from human milk. Only the extremely homologous (98%) turkey lysozyme showed cross-activation, while the other protein effectors were inactive. Therefore, activation is highly specific, and activation by some contaminating factor (salt, magnesium) that might have been introduced during protein preparations is unlikely. In addition, as several of the non-cognate proteins were known to bind RNA both specifically and non-specifically, general stabilization of ribozyme structure by protein ‘salts’ is also an unlikely mechanism for activation.

Nonetheless, it was still possible that contaminants unique to each protein preparation were responsible for activation. In order to discount this source for cross reactivity, the regulatable, catalytically active nucleic acids were incubated with inactivated cognate proteins. Cyt18 was denatured either by heating or by incubation with sodium dodecyl sulfate (SDS), while lysozyme was denatured by a combination of disulfide bond reduction and heating. Denatured Cyt18 was unable to activate ribozyme catalysis, while only lysozyme that had been both reduced and denatured was unable to activate catalysis. Both reduction and denaturation are required to eliminate lysozyme activity. It appeared as though the selected ribozymes were not only specific for their protein effectors, but may also be dependent on protein conformation. In fact, given that anti-peptide antibodies have been shown to partially denature protein structure it may be that protein-activated ribozymes will be found to be even more sensitive to protein conformation than other proteins.

Next, the inventors probed the activation of individual regulatable, catalytically active nucleic acids by using RNA inhibitors of the protein effectors. Previously selected both anti-Cyt18 and anti-lysozyme aptamers were used under buffer conditions similar to those used for these selections. These and other RNA molecules were incubated together with regulatable, catalytically active nucleic acids and their protein effectors, and protein-dependent activation was assessed. Several RNA molecules slightly reduced Cyt18 activation of clone cyt7-2, possibly due to non-specific competition for binding. However, the greatest reduction in activity was observed with RNAs known to bind specifically to Cyt18. The ND1 intron is an in vivo substrate for Cyt18 and shows the greatest reduction in activity, while an aptamer that has been shown to inhibit the ability of Cyt18 to interact with ND1 (M12; Cox and Ellington, unpublished results) was also an effective inhibitor. In contrast, an aptamer that binds to Cycl 8 but does not inhibit its interactions with ND 1 (B17; data not shown) inhibits activation no better than: an anti-lysozyme aptamer (cl), a random sequence pool (N30), or tRNA. Lysozyme activation of its corresponding regulatable, catalytically active nucleic acids (lys11-2) proved to be relatively impervious to all inhibitors except for a high affinity anti-lysozyme aptamer (cl, K_{d}=31 nM), which reduced activation to background levels. The specificity of inhibition observed with these different RNA species further emphasizes the specificity of the interactions between effector proteins and their cognate regulatable, catalytically active nucleic acids.

A direct correlation between the lysozyme binding and ribozyme activation could be demonstrated (FIG. 21). Lysozyme interacts with its regulatable, catalytically active nucleic acids with an apparent K_{d} of 1.5 μM, while the Cyt18 regulatable, catalytically active nucleic, acids could not be saturated even at protein concentrations up to 2.5 μM). Moreover, when the activity of a lysozyme-dependent ribozyme was assayed as a function of salt concentration, binding and catalysis were both depressed by high (1 M) salt concentrations. Interestingly, when the binding of the naive pool was examined, it also bound with a K_{d} of 1.3 μM; the two binding curves were superimposable. Thus, unlike standard aptamer selection in which binding function is necessary for selection, the regulatable, catalytically active nucleic acids of the present invention can be optimized for activation without affecting nascent binding. Given that lysozyme does not in general activate the random pool to any great degree this further emphasizes the specificity of the selected interface.

In natural ribonucleoproteins, protein components activate their nucleic acid counterparts by stabilizing active RNA conformers. The yeast mitochondrial protein CBP2 preferentially stabilizes the active tertiary structure of the intron b15, while Cyt18 assists in folding and stabilization of the ND1 intron. The P-protein of RNase P has been shown to bind near the active site of the ribozyme and to influence substrate specificity. However, unlike ribonuclease P, the function of the protein cofactors of the present invention, nucleoprotein enzymes cannot be replicated by simply increasing monovalent salt concentrations. Therefore the method of the present invention may be used to select regulatable, catalytically active nucleic acids in which activated catalysis is a synergistic property of the modified catalytic domain and its protein ‘cofactor.’ From this vantage, the role of the ribozyme would be to provide an adaptive platform for protein binding.

The ability to select ribozymes that are responsive to protein effectors has important implications for the development of biosensor arrays. The present invention may be used in conjunction with, or as a substitute for identifying antibodies to proteome targets, and are developing antibody-based chips for proteome analysis. However, the performance of such chips is inherently tied to the performance of antibodies. In order to develop sandwich-style assays, at least two different antibodies that recognize nonoverlapping epitopes will need to be identified for each protein target, and the background binding of antibody:reporter conjugates will of necessity limit the sensitivity of ELISA-style assays. In contrast, protein-dependent regulatable, catalytically active nucleic acids could be immobilized on chips, transiently but specifically recognize their protein targets, covalently co-immobilize a reporter conjugated to an oligonucleotide substrate, and then be stringently washed to reduce background. The automation of in vitro selection procedures, as disclosed herein, demonstrate that it is possible to develop high-throughput regulatable, catalytically active nucleic acids selections, which could allow proteome and metabolome targets to be detected and quantitated.

Synthesis of L1-N50 pool and primers. The L1-N50 pool and primers were synthesized using standard phosphoramidite methodologies. Some 424 μg (ca. 10¹⁵ molecules) of the single stranded pool (5′TTCTAATACGACTCACTATAGGACCTCGGCGAAAGC-(N₅₀)-GAGGTTAGGTGCCTCGTGATGTCCAGTCGC (SEQ ID NO:7) T7 promoter underlined, N=A, G, C, or T) was amplified in a 100 ml PCR reaction using the primers 20.T7 (5′-TTCTAATACGACTCACTATA) (SEQ ID NO:8) and 18.90a (5′ GCGACTGGACATCACGAG) (SEQ ID NO:9). The substrate used in the selection was S28A-biotin (biotin-(dA)₂₂-ugcacu; RNA in lowercase). A non-biotinylated version of this substrate (S28A) was used in most ligation assays. During selection, a selective PCR primer set, 28A. 180 (5′ (dA)₂₂-TGCACT)/18.90a, was used to amplify ligated ribozymes. A degenerative PCR primer set, 36AB.2 (5′TTCTAATACGACTCACTATAGGACCTCGGCGAAAGC) (SEQ ID NO: 10)/18.90a, restored the T7 promoter to the selected pool in preparation for further rounds of transcription and selection.

In vitro selection of protein dependent ribozymes. Briefly, pool RNA (5 μM) and 18.90a (10 μM) were first denatured in water. Ligation buffer (50 mM Tris, pH 7.5, 100 mM KCl, 10 mM MgCl₂) and substrate oligonucleotide (S28A-biotin, 10 μM) were then added in the absence of the target protein (except round 1). After this negative (−) incubation at 25° C., the selection mixture was segregated using a streptavidin-agarose resin (Fluka, Switzerland) to capture biotinylated substrate, free or ligated to the ribozyme. The eluant containing unligated ribozymes was collected and a second, positive (+) incubation was initiated by the addition of target protein (10 μM) and additional substrate (S28A-biotin, 10 μM). Following incubation at 25° C. the mixture was again segregated using streptavidin-agarose. The resin containing ligated ribozymes was washed thoroughly and then suspended in RT buffer (50 mM Tris, pH 8.3, 75 mM KCl, 3 mM MgCl₂, 10 mM DTT, 400 μM dNTPs, 5 μM 18.90a) and reverse transcribed using SuperScript II reverse transcriptase (Gibco BRL, Gaithersburg, Md.). The CDNA molecules in the resin slurry were then PCR amplified using first the selective primer set and then the regenerative primer set. The final PCR product was transcribed using T7 RNA polymerase (Epicentre, Madison, Wis.). Stringency was steadily increased over the course of the selection by decreasing the (positive selection) ligand incubation times and increasing the (negative selection) ligand incubation times (See FIGS. 18A and 18C).

Ligation assays. In one example, 10 pmol of [³²P]-body-labeled ribozyme and 20 pmol effector oligonucleotide were denatured for 3 min at 70° C. in 5 μl water. The RNA mixture was cooled to room temperature followed by addition of ligation buffer and target protein (20 pmol unless otherwise stated, or water in place of ligand, in the case of ligand samples). After a 5 min equilibration at room temperature, reactions were initiated by the addition of 20 pmol substrate oligonucleotide (S28A) in a final volume of 15 μl. Reactions were incubated at 25° C., and 4 μl aliquots were removed at three appropriate time points and terminated by the addition of 18 μl of SDS stop mix (100 mM EDTA, 80% formamide, 0.8% SDS, 0.05% bromophenol blue, 0.05% xylene cyanol). Samples were denatured for 3 min at 70° C., ligated and unligated species were separated from one another on 8% polyacrylamide gels containing 0.1% SDS, and the amounts of products formed were determined using a Phosphorimager (Molecular Dynamics, Sunnyvale, Calif.). Assays performed over a broad range of protein concentrations (e.g, FIG. 21) differed from typical reaction conditions in that only 1 pmol ribozyme was present in a 10 μl final volume.

Protein inactivation. Standard ligation assays were performed as described above, but in the presence of protein samples that had been pre-treated as follows. Cyt18 protein was denatured by heating for 10 min at 70° C. or by the addition of 6% SDS (0.7% SDS in ligation reaction). Lysozyme was heated 10 min at 100° C. or incubated 10 min at room temperature in the presence of 2 mM DTT (0.3 mM DTT final reaction) without inactivating the protein. The protein was successfully inactivated by heating for min at 70° C. in the presence of 2 mM DTT. Ligation reactions were performed with 1.3 μM protein in 15 μl reactions incubated 5 min at 25° C.

Competition assays. Ligation assays were performed as described above, using 10 pmol of [³²P]-body-labeled ribozyme (cyt7-2 or lys11-2; 1 &M) and 20 pmol effector oligonucleotide (2 μM). The denatured and annealed RNA mixture was combined with ligation buffer, 20 pmol protein (Cyt18, lysozyme, or water in the case of (−) protein samples; 2 μM), and 30 pmol of denatured and annealed competitor RNA (3 μM). Competitor RNAs are as follows:

| | |
|---|---|
| M12 | |
| GGGAA UGGAU CCACA UCUAC GAAUU CGAGU CGAGA ACUGG UGCGA | (SEQ ID NO:11) |
| AUGCG AGUAA GUUCA CUCCA GACUU GACGA AGCUU), | |
| B17 | |
| GGGAA UGGAU CCACA UCUAC GAAUU CGUAG CGUAG AGUAU | (SEQ ID 10 NO:12) |
| GAGAG AGCCA AGGUC AGGUU CACUC CAGAC UUGAC GAAGC UU) | |
| cl | |
| GGGAA UGGAU CCACA UCUAC GAAUU CAUCA GGGCU AAAGA GUGCA | (SEQ ID NO:13) |
| GAGUU ACUUA GUUCA CUCCA GACUU GACGA AGCUU | |
| ND1 | |
| GACUA AUAUG AUUUG GUCUC AUUAA AGAUC ACAAA UUGCU | (SEQ ID NO:14) |
| GGAAA CUCCU UUGAG GCUAG GACAA UCAGC AAGGA AGUUA ACAUA | |
| UAAUG UUAAA ACCUU CAGAG ACUAG ACGUG AUCAU UUAAU AGACG | |
| CCUUG CGGCU CUUAU UAGAU AAGGU AUAGU CCAAA UUUGU AUGUA | |
| AAUAC AAAAU GAUAA AAAAA AAUGA AAUCA UAUGG G | |
| N30 | |
| GGGAA UGGAU CCACA UCUAC GAAUU C-N30-U UCACU CCAGA | (SEQ ID NO:15) |
| CUUGACGAAG CUU | |

Where N=(A, G, C, U), and tRNA (from Yeast; Gibco BRL, Gaithersburg, Md.). Reactions were incubated 5 min at 25° C. and initiated by the addition of 20 pmol substrate oligonucleotide (S28A; 2 μM) in a final volume of 10 μl. Cyt18 reactions were incubated 5 min at 25° C. and lysozyme reactions were incubated 10 min. Reactions were terminated by the addition of 45 μl of SDS/urea stop mix (75 mM EDTA, 80% formamide, saturated urea, saturated SDS, 0.05% bromophenol blue, 0.05% xylene cyanol) and analyzed on 8% polyacrylamide gels containing 0.1% SDS as above.

Binding assays. Binding assays were performed in triplicate by combining I pmol of [³²P]-body-labeled RNA, 20 pmol 18.90a, and varying amounts of target protein (1 pmol to 5 nmol) in 50 μl of ligation buffer. After incubation at room temperature for 30 min, the mixture was drawn under vacuum through a series of nitrocellulose and nylon filters and washed with 150 μl of ligation buffer. The ratio of protein-bound RNA versus free RNA was determined by analyzing the counts retained on the nitrocellulose filter versus the counts on the nylon filter.

In FIG. 17, L1 ligase, L1-N50 pool, and selection scheme. FIG. 17(a) shows the L1 ligase was the starting point for pool design. Stems A, B, and C are indicated. The shaded region indicates the catalytic core and ligation junction. Primer binding sites are shown in lower case, an oligonucleotide effector required for activity is shown in italics, and the ligation substrate is bolded. The ‘tag’ on the ligation substrate can be varied, but throughout this selection was biotin-(dA)₂₂. FIG. 17(b) shows the L1-N50 pool contains 50 random sequence positions and overlaps with a portion of the ribozyme core. Stem B was reduced in size and terminated with a stable GNRA tetraloop, and position U5 of stem A was mutated to a C (in bold) to form a base pair with G69 to increase the stability of the stem. FIG. 17(c) shows one selection scheme of the present invention. The RNA pool was incubated with a biotinylated substrate and reactive variants were removed from the population. The remaining species were again incubated with a biotinylated substrate in the presence of the target protein (Cyt18 or lysozyme). Reactive variants were removed from the population and preferentially amplified by reverse transcription, PCR, and in vitro transcription.

FIG. 18 shows the progression of the L1-N50 selections. FIG. 18(a) shows the conditions for the selection of Cyt18-dependent ribozymes. The ‘substrate’ column charts the molar excess of substrate to ribozyme. FIG. 18(b) shows the progress of the L1-N50 Cyt18 selection. Ligation rates for each round of selection are plotted as black bars for assays performed in the presence of Cyt18 and gray bars for assays in the absence of Cyt18. The gray line the level of activation by Cyt18 and is measured against the right-hand axis.

FIG. 18(c) and 18(d) show the conditions for the selection of lysozyme-dependent ribozymes and the L1-N50 lysozyme selection. Graphing conventions are as in FIG. 18b.

FIG. 19 shows protein-dependent regulatable, catalytically active nucleic acid sequences and structures. FIG. 19(a) shows the sequences of the ribozyme N50 regions. Cyt18-dependent clones are indicated by the prefix “cyt” and lysozyme dependent clones are indicated by the prefix ‘ys’. The number following these prefixes indicates the round from which the ribozyme was cloned (e.g., cyt7-2 was from the 7th round of selection). The frequency that a given motif appears (out of 36 ‘cyt’ clones and 24 ‘lys’ clones) in the sequenced population is indicated in parentheses. Regions of sequence similarity between individual clones are boxed. FIG. 19(b) is a hypothetical secondary structure of the dominant Cyt18-dependent clone cyt7-2.

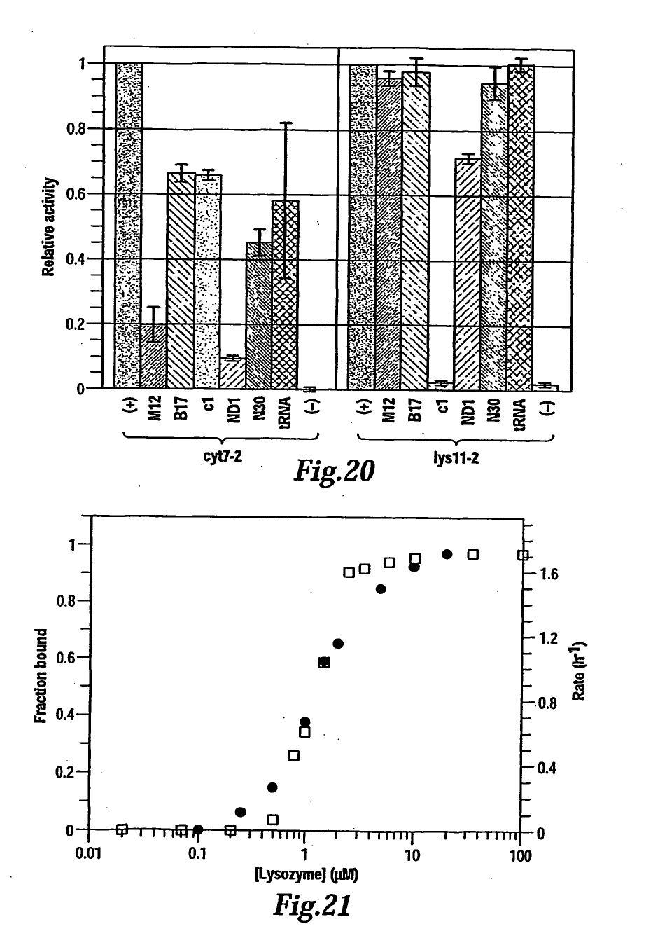

FIG. 20 demonstrates the ribozyme activity with inactivated protein samples. Ligation assays for the Cyt18-dependent clone cyt9-18 and the lysozyme-dependent clone lys11-2 were performed in the presence of treated Cyt18 and lysozyme, respectively.

FIG. 21 demonstrates an aptamer competition assays. Relative ligation activity of cyt7-2 and lysl 1-2 assayed in the presence of various specific and non-specific aptamer and RNA constructs. Samples labeled (+) contain activating protein with no competitor, while samples labeled (−) do not contain protein. The other samples contain either aptamers for Cyt18 (M12, B17) or lysozyme (c1), a group I intron that binds Cyt18 (ND1), or other non-specific RNAs as described in the text. FIG. 21 shows the binding and ligation activity as a function of protein concentration. Fraction of lys11-2 RNA bound to lysozyme (open squares (G), left-hand axis) superimposed onto the reaction rate of lys11-2 RNA (closed circles (J), right-hand axis) over a range of lysozyme concentrations.

### EXAMPLE 4: PEPTIDE SPECIFIC REGULATABLE, CATALYTICALLY ACTIVE NUCLEIC ACIDS

Rev-dependent RNA ligase ribozymes. An L1-N50 pool (10¹⁵ starting species) was subjected to an iterative regime of negative and positive selections for ligation activity. The pool was initially incubated with a biotinylated substrate and reactive species were removed; the pool was then mixed with the effector molecule, a 17 amino acid fragment of the HIV Rev protein, and reactive species were removed and amplified. The Rev peptide is a highly basic arginine rich motif whose natural function is as an RNA binding domain. In addition, RNA aptamers to the full Rev protein and the 17mer Rev peptide have been isolated using in vitro selection. During the course of the study the stringency of the negative selections was increased by increasing the time allowed for ligation and substrate concentration in the absence of Rev peptide. The stringency of the positive selection step was increased by decreasing the time allowed for ligation and the substrate concentration.

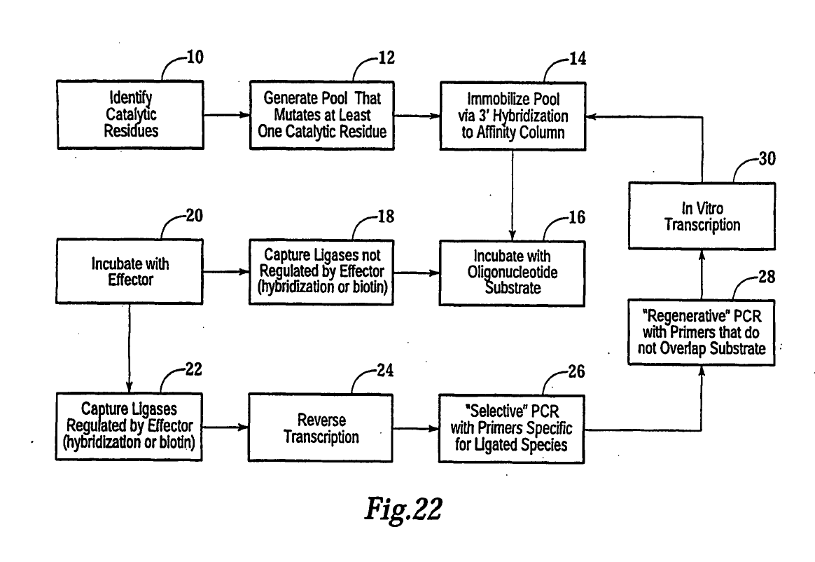

FIG. 22 is a flow chart of a method for negative and positive selection of RCANA according to the present invention. In step 10, the catalytic residues of a catalytic nucleic acid are identified. Next, a pool of oligonucleotides is generated in which at least one residue in the catalytic domain is mutated (step 12). In step 14, the pool of oligonucleotides is immobilized via, e.g., 3′ hybridization to an affinity column followed by incubation of the immobilized oligonucleotide pool (step 16) with the cognate substrate of the catalytic residues. In the case of ligases, for example, those mutated pool members that maintain activity without the presence of an effector are removed from the pool (step 18). Step 18 is the negative selection step and the stringency may be increased or decreased by changing, e.g., the length of time of exposure between the enzyme and the ligand, salt and temperature conditions, buffers and the like. The remaining mutated members of the pool are incubated with an effector in step 20, which is the positive selection step for RCANA. The stringency of positive selection may also be affected by changing, e.g, the length of time of exposure between the enzyme and the ligand, salt and temperature conditions, buffers and the like. The pool members that become active, or more active, upon exposure to the effector in step 22 are removed, e.g., using capture ligases, the sequences are reverse transcribed in step 24 and isolated using, e.g., PCR using selective oligonucleotides for ligated species. These RCANA may be further selected and improved through subsequent rounds of selection, which may include the use of regenerative oligonuCleotides that do not overlap the substrate binding portion of the RCANA followed by in vitro transcription and reintroduction into the system at, e.g, step 14.

TABLE 1

| | (−) incubation | | (+) incubation | |
|---|---|---|---|---|
| Round | substrate | (−) Cyt18 | substrate | (+) Cyt18 |
| 1 | | | 2X | 6 h |
| 2 | 2X | 24 h | 2X | 16 h |
| 3 | 2X | 24 h | 2X | 5 h |
| 4 | 2X | 24 h | 2X | 30 min |
| 5 | 2X | 48 h | 2X | 5 min |
| 6 | 2X | 95 h | 2X | 5 min |
| 7 | 2X | 95 h | 2X | 1 min |
| 8 | 2X | 95 h | 2X | 30 sec |
| 9 | 5X | 94 h | 2X | 30 sec |

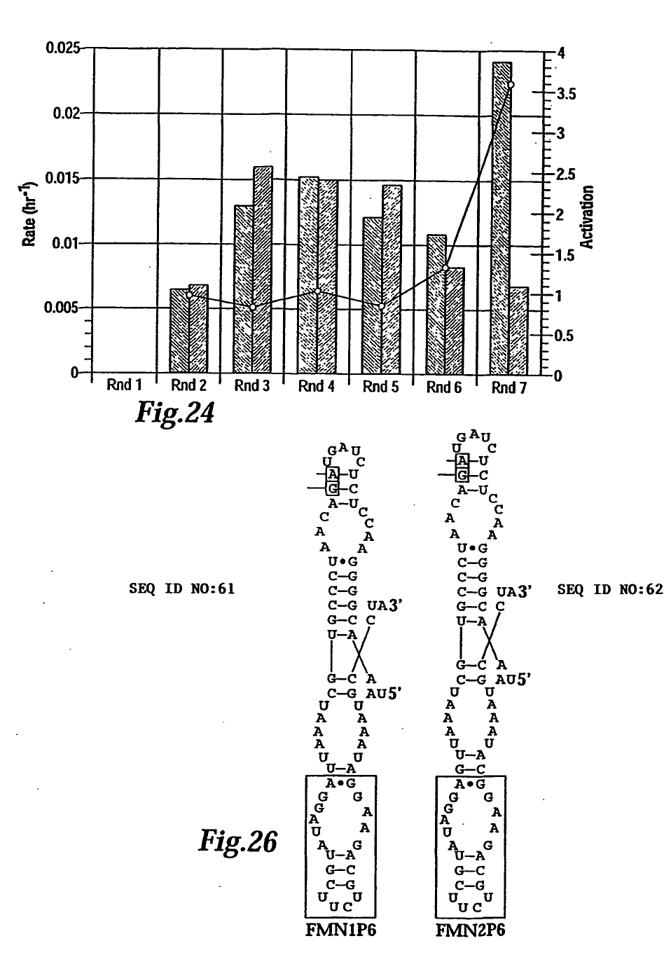

The degree of peptide-dependent activation was assessed in a standard ligation assay. Ligation activity independent of the presence of Rev peptide progressively increased through Round 6 (FIG. 24). By Round 7, the standard kinetic analysis of the population began to display two distinct phases indicating potentially that at least two different species of catalyst with different characteristics were becoming predominant in the population. The first phase indicated a population with fast ligation rate but which was not affected by the presence of peptide. The second phase indicated a population that was about 60-fold slower than the first phase population but which did show a small degree of peptide activation.

Two additional rounds of selection were performed with increased stringency in the negative selection and the final two rounds of the selection were cloned and sequenced. Kinetic analysis of the individual isolates revealed that the initial peptide insensitive phase of the kinetic analysis could be contributed to a single clone (R8-1), which ligates: with a fast rate (52 h⁻¹) independent of the presence of peptide. Clone R8-1 is nearly identical to a ribozyme (JH1). A second clone (R8-4) showed Rev peptide induced activation. Clone R84 performed the ligation reaction with an observed rate of 0.86 h⁻¹ in the presence of Rev peptide, but this rate dropped to 0.000046 h⁻¹ when the peptide was left out of the reaction, a difference of 18,600-fold. Interestingly, the remaining four clones that were sequenced (including clone R8-2), which accounted for 65% of the final population, were completely inactivve in the standard ligation assay. Additionally, when these clones were assayed in the presence of the round 9 pool RNA, ligation activity remained undetectable, eliminating the possibility that these clones are persisting in the population by using a parasitic trans-ligation mechanism in which substrate is ligated onto these RNAs by some other ligase in the mixture in a transligation reaction.

Specificity of activation. In order to assess the specificity of activation of selected ribozymes by peptide effectors, the Rev-dependent ligase was incubated with a variety of peptides, including HIV Tat, BIV Tat, bREX, bradykinin, as well as arginine. Activation was observed only with HIV Tat peptide at about 30%. In addition, the complete Rev protein was able to activate the ligase about 10% as well as the peptide. The ligase was assayed in the presence of different preparations of Rev peptide with different capping structures. All preparations of the Rev peptide activate the ligase but to slightly different extents. The selection was performed with a capped peptide (sREVn) that increases the degree of a-helicity of the peptide to mimic its conformation in the full Rev protein. A less capped peptide (aREV) with less a-helical character than sREVn was the best activator by about a factor of 2. These results suggest that activation is highly specific and not due to some contaminating factor (salt, magnesium) that might have been introduced during a particular peptide preparation. In addition, as several of the non-cognate peptides were known to bind RNA, both specifically and non-specifically, general stabilization of ribozyme structure by protein ‘salts’ was an unlikely mechanism for activation.

To further eliminate the possibility that some non-peptide contaminant of the peptide preparations was the actual activator of the ligase, the peptide was treated to destroy the peptide and then assayed to see if the sample could still activate the ligase. Peptide was treated with either a standard acid hydrolysis or a trypsin digestion. Neither treated peptide sample was able to activate the ribozyme.

Synthesis of L1-N50 pool and primers. The L1-N50 pool and primers were synthesized using standard phosphoramidite methodologies. Some 424 μg (ca. 1015 molecules) of the single stranded pool (5′TTCTAATACGACTCACTATAGGACCTCGGCGAAAGC-(N₅₀)-GAGGTTAGGTGCCTCGTGATGTCCAGTCGC (SEQ ID NO:7) T7 promoter underlined, N=A, G, C, or T) was amplified in a 100 ml PCR reaction using the primers 20.T7 (5′-TTCTAATACGACTCACTATA) (SEQ ED NO:8) and 18.90a (5′GCGACTGGACATCACGAG) (SEQ ID NO:9). The substrate used in the selection was S28A-biotin (biotin-(dA)₂₂-ugcacu; RNA in lowercase). A non-biotinylated version of this substrate (S28A) was used in most ligation assays. During selection, a selective PCR primer set, 28A.180 (5′ (dA)₂₂-TGCACT)/18.90a, was used to amplify ligated ribozymes. A regenerative PCR primer set, 36.dB.2 (5′TTCTAATACGACTCACTATAGGACCTCGGCGAAAGC) (SEQ ID NO: 10)/18.90a, restored the T7 promoter to the selected pool in preparation for further rounds of transcription and selection.

In vitro selection of peptide dependent ribozymes. The selection procedure for protein dependent ligase ribozymes has been described herein above. Briefly, pool RNA (5 μM) and 18.90a (10 μM) were first denatured in water. Ligation buffer (50 mM Tris, pH 7.5, 100 mM KCl, 10 mM MgCl₂) and substrate oligonucleotide (S28A-biotin, 10 μM) were then added in the absence of the target protein (except round 1). After this negative (−) incubation at 25° C., the selection mixture was segregated using a streptavidin-agarose resin (Fluka, Switzerland) to capture biotinylated substrate, free or ligated to the ribozyme. The eluant containing unligated ribozymes was collected and a second, positive (+) incubation was initiated by the addition of target protein (10 μM) and additional substrate (S28A-biotin, 10 μM). Following incubation at 25° C. the mixture was again segregated using streptavidin-agarose. The resin containing ligated ribozymes was washed thoroughly and then suspended in RT buffer (50 mM Tris, pH 8.3, 75 mM KCl, 3 mM MgCl₂, 10 mM DTT, 400 μM dNTPs, 5 μM 18.90a) and reverse transcribed using SuperScript II reverse transcriptase (Gibco BRL, Gaithersburg, Md.). The cDNA molecules in the resin slurry were then PCR amplified using first the selective primer set and then the regenerative primer set. The final PCR product was transcribed using T7 RNA polymerase (Epicentre, Madison, Wis.). Stringency was steadily increased over the course of the selection by decreasing the ligand incubation times (positive selection) and increasing the ligand incubation times (negative selection) (See Table 1).

Ligation assays. Ligation assays were performed as described hereinabove. Typically, 10 pmol of [³²P] -body-labeled ribozyme and 20 pmol effector oligonucleotide were denatured for 3 min at 70° C. in 5 μl water. The RNA mixture was cooled to room temperature followed by addition of ligation buffer and target peptide (20 pmol unless otherwise stated, or water in place of ligand, in the case of (−) ligand samples). After a 5 min equilibration at room temperature, reactions were initiated by the addition of 20 pmol substrate oligonucleotide (S28A) in a final volume of 15 μl. Reactions were incubated at 25° C., and 4 μl aliquots were removed at three appropriate time points and terminated by the addition of 18 pi of SDS stop mix (100 mM EDTA, 80% formamide, 0.8% SDS, 0.05% bromophenol blue, 0.05% xylene cyanol). Samples were denatured for 3 min at 70° C., ligated and unligated species were separated from one another on 8% polyacrylamide gels containing 0.1% SDS, and the amounts of products formed were determined using a Phosphorimager (Molecular Dynamics, Sunnyvale, Calif.). Assays performed over a broad range of peptide concentrations differed from typical reaction conditions in that only 1 pmol ribozyme was present in a 10 μl final volume.

Peptide inactivation. Standard ligation assays were performed as described above, but in the presence of peptide samples that had been pre-treated as follows. Peptide (15 nmol) was either hydrolyzed for 24 h in 6 M HCl at 100° C. or digested with trypsin-immobilized agarose resin 14 h at 37° C. Both samples were evaporated to dryness and resuspended in water to a final concentration of 150 μM and used in place of peptide in standard ligation assays. In addition, control samples for hydrolysis and trypsin digestion containing no peptide were treated as described for peptide samples and tested to insure that they did not inhibit ligation in the presence of intact peptide.

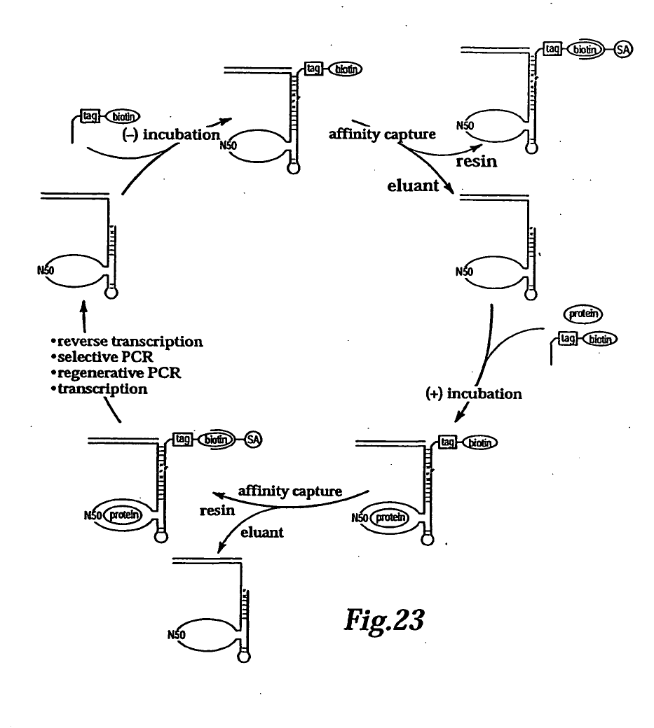

FIG. 23 shows the selection scheme for peptide binding. The RNA pool was incubated with a biotinylated substrate and reactive variants were removed from the population. The remaining species were again incubated with a biotinylated substrate in the presence of the target peptide. Reactive variants were removed from the population and preferentially amplified by reverse transcription, PCR, and in vitro transcription.

FIG. 24 shows the progress of the L1-N50 Rev selection. Ligation rates for each round of selection are plotted as black bars for assays performed in the presence of Rev peptide and gray bars for assays in the absence of Rev peptide. The gray line indicates the level of activation by Rev peptide and is measured against the right-hand axis. The ‘substrate’ column charts the molar excess of substrate to ribozyme.

### EXAMPLE 5: IN VIVO GENE REGULATION USING REGULATABLE, CATALYTICALLY ACTIVE NUCLEIC ACIDS

The present invention also includes the design and isolation of regulatable, catalytically active nucleic acids generated in vitro by design and selection for use in vivo.

The regulatable, catalytically active nucleic acids disclosed herein permit the control of gene regulation or viral replication in vivo. The present inventors have generated regulatable, catalytically active nucleic acids that allow directed, in vivo splicing controlled by exogenously added small molecules. Substantial differences in gene regulation were observed with compounds that differed by as little as a single methyl group. Regulatable, catalytically active nucleic acids are used as genetic regulatory switches for generating conditional knockouts at the level of mRNA or for developing economically viable gene therapies.

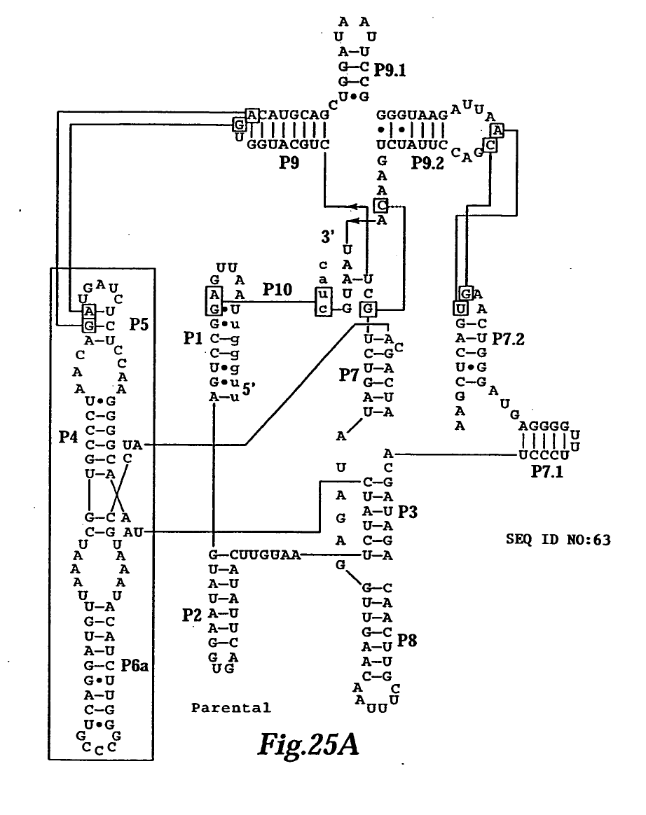

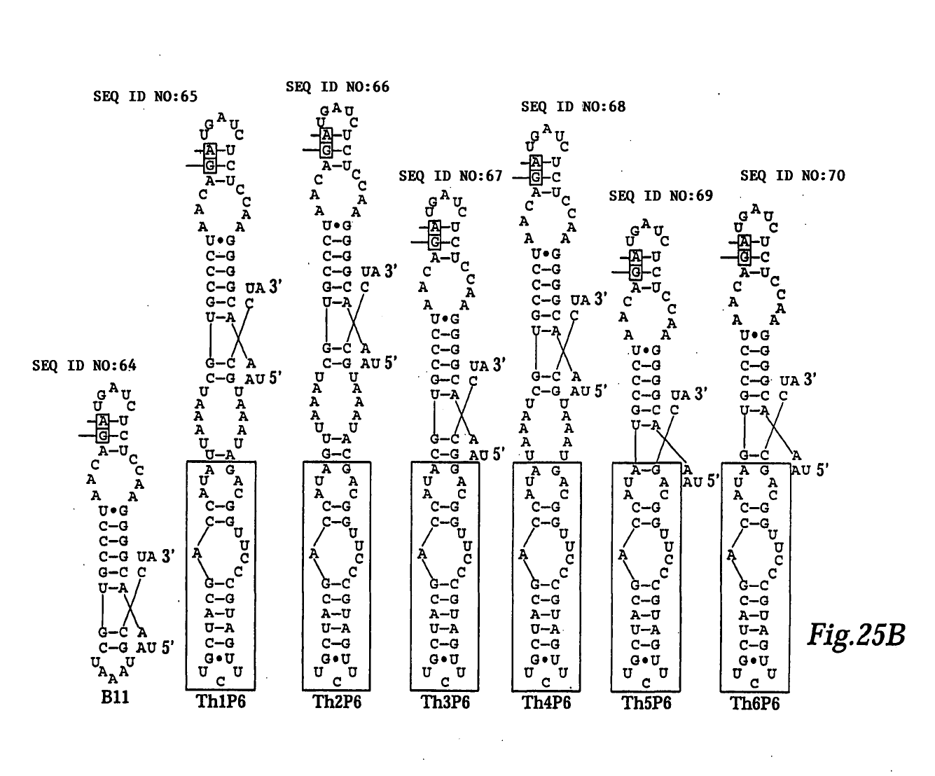

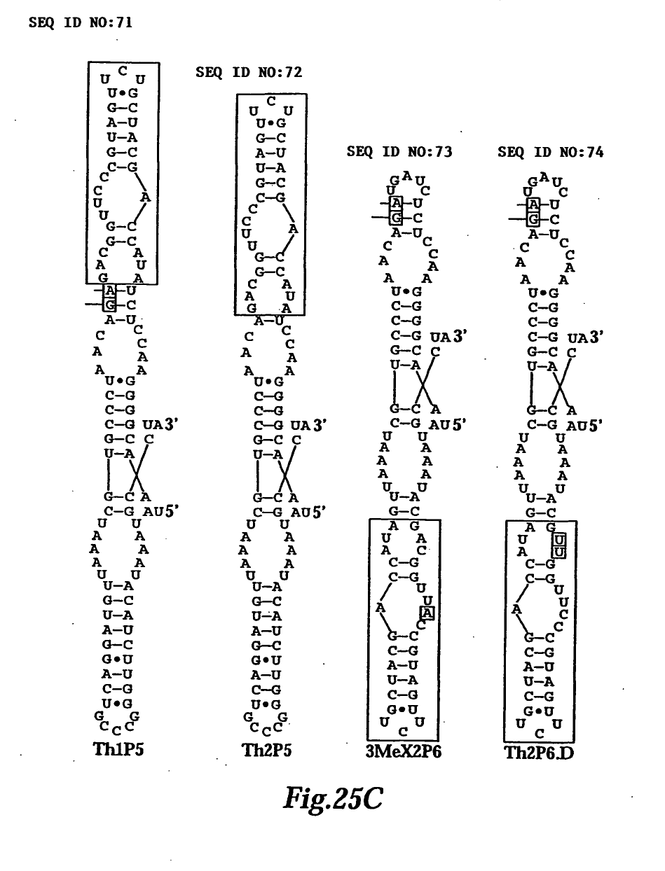

In order to convert the Group I self-splicing intron into a regulatable, catalytically active nucleic acid, it was necessary to first identify sequences or structures in the catalytic domain of a ribozyme whose conformation might modulate splicing activity. One example of a ribozyme catalytic domain that may be used with the present invention is the Group I self-splicing intron because its structural and kinetic properties and interaction with the thymidylate synthase (td) gene in bacteriophage T4 have been extensively studied. A series of nested deletions of the P6 stem-loop partially or completely compromise ribozyme activity. More importantly, either magnesium or the tyrosyl tRNA synthetase from Neurospora mitochondria (CYT-18) can suppress many of these defects. Other introns have also revealed that deletion of the P5 stem-loop can modulate ribozyme activity. The present inventors recognized that sites where deletions modulated ribozyme activity might also prove to be sites where conformational changes to a nucleic acid may modulate catalytic activity. A series of Group I aptazymes were designed in which the anti-theophylline aptamer was substituted for either a portion of P6 or P5 (FIG. 25). The point of attachment of the anti-theophylline sequence was selected for the design of theophylline-dependent cleavases and ligases.

The self-splicing activities of the Group I, regulatable, catalytically active nucleic acids were examined in vitro using a standard splicing assay. The stringency of ligand-induced suppressions of splicing defects was examined by carrying out the reactions at either low (3 mM, stringent) or high (8 mM, permissive) magnesium concentrations. Several of the constructs were inactive (e.g., Th3P6, Th5P6, and Th6P6) or showed no differential splicing activity (e.g., Th4P6 and Th2P5), but four constructs, Th1P6, Th2P6, Th3P6, and Th1P5, showed increased self-splicing in the presence oftheophylline. For all of the nucleic, acids except Th3P6, the ligand-induced splicing activity was greater in a standard assay at the more stringent magnesium concentration (See Table 2 below).

Table 2 shows the relative in vitro splicing activity of constructs containing anti-theophylline aptamers. Extent of reaction is relative to the parental construct in 3 mM MgCl₂ with no theophylline at 2 h.

TABLE 2

| [MgCl₂] | |
|---|---|
| 3 mM | 8 mM |

| [Theo] | 1.5 mM | 0 mM | 1.5 mM | 0 mM |
|---|---|---|---|---|
| Parental | 0.85 | 1.00 | 0.61 | 0.68 |
| B11 | 0.03 | 0.02 | 0.31 | 0.34 |
| Th1P6 | 0.05 | 0.20 | 0.31 | 0.16 |
| Th2P6 | 0.04 | 0.15 | 0.31 | 0.04 |
| Th3P6 | 0.03 | 0.04 | 0.2 | 0.04 |
| Th4P6 | 0.05 | 0.06 | 0.38 | 0.37 |
| Th5P6 | 0.04 | 0.00 | 0.05 | 0.03 |
| Th6P6 | 0.03 | 0.01 | 0.00 | 0.03 |
| Th1P5 | 1.08 | 0.91 | 0.85 | 0.74 |
| Th2P5 | 0.70 | 0.57 | 0.03 | 0.03 |

The construct Th3 P6 was inactive at lower magnesium concentrations, and the more permissive concentration was required to observe ligand-induced splicing activity. Interestingly, those constructs that showed ligand-dependent activity closely resembled the original deletion variants that showed magnesium-dependent recovery of splicing activity. For example, the junction between the binding and the Group I catalytic domain in the activatable regulatable, catalytically active nucleic acids Th2P6 resembled the construct td P6-6 whose splicing defect at 3 mM magnesium was suppressed by 8 mM magnesium or by stabilization of the capping tetraloop sequence. Defects that poise a ribozyme between active and inactive conformers have previously been used to engineer effector-dependence.

Next, the extent of ligand-dependent activation was determined by examining the kinetics of splicing in the presence and absence of theophylline. The nucleic acid modified at P5 (Th1P5) showed very little (1.6-fold) activation. Nucleic acids modified at P6 showed somewhat greater activation, with Th2P6 yielding 9-fold activation and Th1P6 18-fold initial rate enhancement in the presence of theophylline. These levels of ligand-dependent activation were similar to those observed with the hammerhead ribozyme constructs, and it may prove possible to use in vitro selection to further optimize activation using the materials and methods of the present invention.

The mechanism of activation on the nucleic acids disclosed herein is likely the same as has been observed for other nucleic acids: ligand-induced conformational changes that stabilize functional nucleic acid sequences and structures. However, the Group I self-splicing intron is a much more complicated ribozyme than either the hammerhead or the L1 ligase; for example, the tertiary structure of the Group I intron is established by a complicated folding pathway. Therefore, it was possible that theophylline-binding influenced the overall folding or stability of the engineered Group I aptazyme, rather than merely altering the local conformation of a functional structure. In order to assess this possibility the theophylline-dependence of splicing reactions in vitro was examined following prolonged incubation to allow re-folding and initiation of catalysis with exogenous GTP. No change in the degree or rate of ligand-dependent activation was observed following pre-incubation. Similarly, when theophylline was added to an in vitro splicing reaction that had previously been initiated with GTP, an increase in the rate of splicing to levels previously observed in the presence of theophylline was observed. Taken together, these results militate against the assumption that theophylline influences the folding pathway of the engineered Group I aptazymes.

An attempt was made to change the effector specificity of the Group I aptazyme by changing which aptamer sequence was conjoined to the catalytic core. Previous studies with both the native hammerhead ribozyme and the L1 ligase showed that such swaps of allosteric binding sites and effector specificities were frequently possible.

Soukup, G. A.& Breaker, R. R. Engineering precision RNA molecular switches. Proc. Natl. Acad Sci. U.S.A. 96, 3584-3589 (1999), and Robertson, M. P.& Ellington, A. D. Design and optimization of effector-activated ribozyme ligases. Nucleic Acids Res 28, 1751-1759 (2000). To this end, the two most successful P6 constructs, Th1P6 and Th2P6, were re-engineered so that the anti-FMN aptamer was inserted in place of the anti-theophylline aptamer. The point of attachment of the anti-FMN aptamer was the same as had previously proven successful in the design of other FNW-dependent ribozymes (FIG. 26). Both flavin-sensing Group I aptazymes were activated by FNW in a standard assay as well as or better than the theophylline-sensing Group I aptazymes. This result is especially significant given that FNIN inhibits Group I splicing activity (albeit at concentrations higher than disclosed herein). Similar specificity swaps were attempted with anti-ATP and anti-HIV-1 Rev binding sequences, but neither of these potential allosteric binding sites appeared to communicate with the catalytic core of the intron. The anti-FMN aptamer may have been more readily substituted for the anti-theophylline aptamer because both terminate in an A:G base-pair. A different connecting stem or ‘communication module’ may allow the melding of other allosteric domains with the Group I ribozyme.

In Table 3, the relative in vitro splicing activity of constructs containing anti-FMN aptamers is shown. The extent of reaction is relative to the parental construct in 3 mM MgCl₂ with no FMN at 2 h.

TABLE 3

| [MgCl₂] | |
|---|---|
| 3 mM | 8 mM |

| [FMN] | 1 mM | 0 mM | 1 mM | 0 mM |
|---|---|---|---|---|
| Parental | 0.84 | 1.00 | 0.89 | 0.79 |
| B11 | 0.14 | 0.05 | 0.08 | 0.5 |
| FMN1P6 | 0.08 | 0.61 | 0.56 | 0.65 |
| FMN2P6 | 0.06 | 0.41 | 0.44 | 0.19 |

Each of the successful nucleic acid constructs disclosed herein was subsequently cloned into an interrupted thymidylate synthetase gene in place of the parental td self-splicing intron. The vectors were introduced into an E. coli strain (C600ThyA::Kan^{R}) that lacked a functional thymidylate synthetase gene and that were thymidine auxotroph. When bacteria grown in rich media were subsequently plated on minimal media lacking thymidine, no colony growth was observed with the exception of Th1P5. However, when theophylline (7.5 mM) was included in the minimal media, colony growth was observed for the intron Th2P6 and increased growth for Th1P5. Interestingly, no growth was observed for constructs harboring the intron Th1P6, despite the fact that this nucleic acid showed a much greater level of theophylline-enhanced splicing in vitro. All introns that originally showed no or low splicing in vitro (including Th3P6) could not rescue cells either in the presence or absence of theophylline. Finally, no growth was observed in a negative control that contained a non-functional Group I intron (B11) and no growth change in a negative control in which mutations were introduced to abolish theophylline binding (Th2P6.D) either in the presence or absence of theophylline.

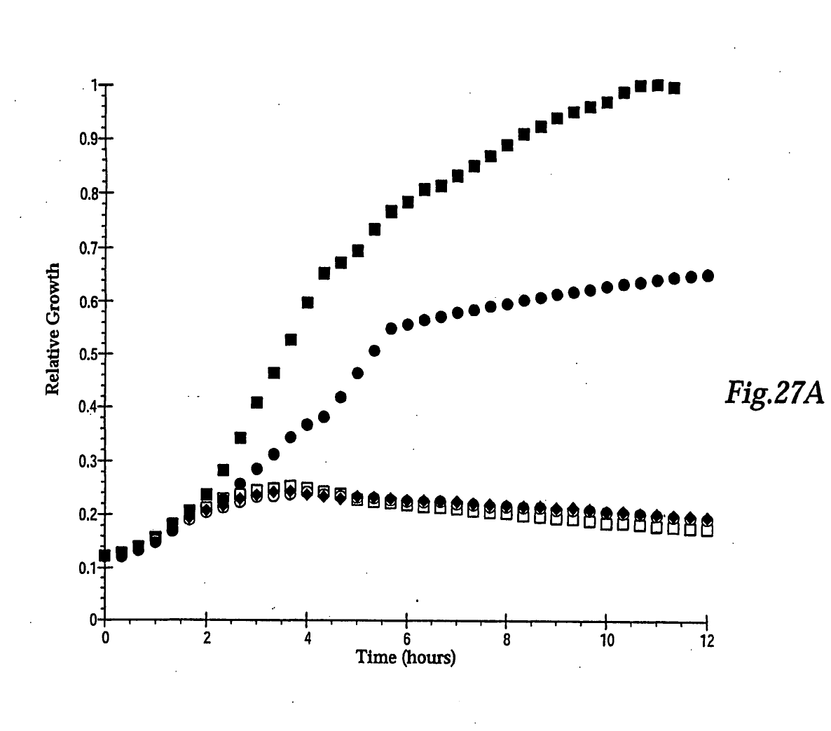

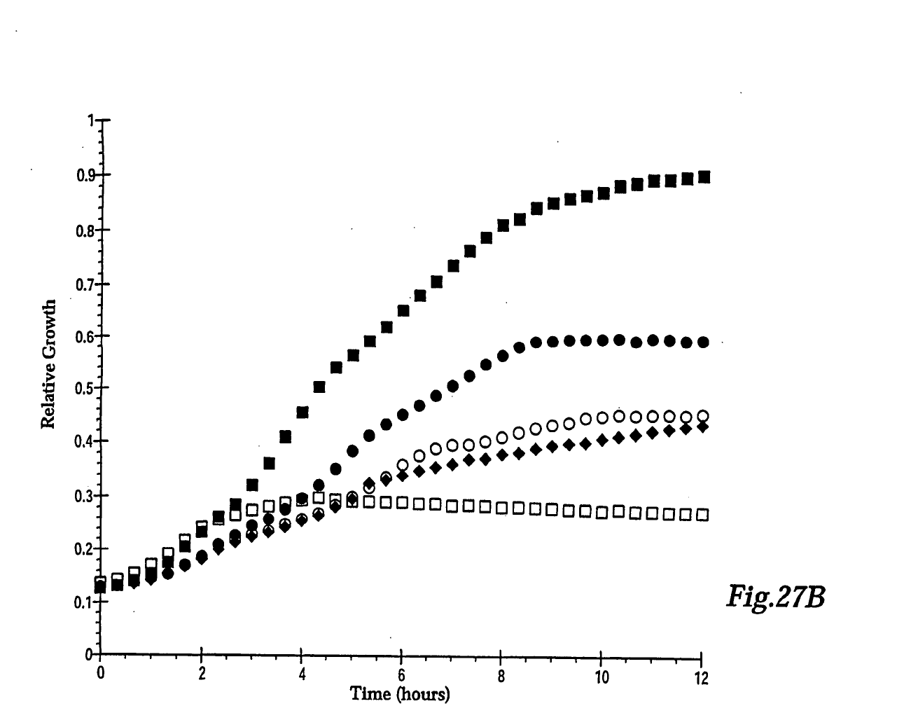

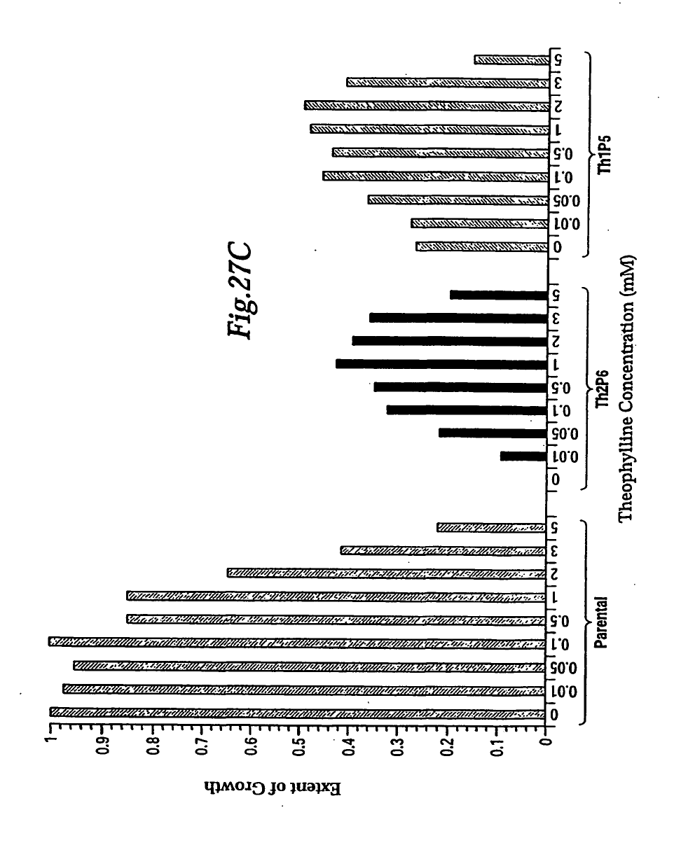

To better quantitate the influence of the effector on intron-splicing, growth experiments in liquid culture were conducted (FIG. 27(a)). An overnight culture that contained the td gene divided by the nucleic acid Th2P6 was inoculated into fresh, minimal media, effector was added, and the resultant growth curves were continuously monitored. As expected based on the results from growth assays on solid media, little growth is observed in the absence of theophylline. However, when theophylline (0.5 mM) is added to liquid medium, cells grow almost as well as a control in which the parental intron is inserted into the td gene.

Importantly, cell growth is not activated by the structurally-related effector caffeine (i.e., 7-methyltheophylline), and no effector-dependent growth differences are observed with cultures containing td genes divided by the non-functional Group I intron B11. The anti-theophylline aptamer is known to discriminate between caffeine and theophylline by a factor of 10,000-fold. Similar results were obtained with cultures that contained the td gene divided by the nucleic acid ThiP5 (FIG. 27(b)). However, in this instance there was some background growth of uninduced cells, consistent with the higher level of background splicing activity in vitro. If theophylline is regulating intron splicing in vivo, then the extent of cell growth should be dependent upon the concentration of theophylline introduced into the media (FIG. 27(c)). Theophylline was toxic to cells, and caused a decrease in the (growth of cells containing the parental td intron at concentrations greater than 0.5 mM. Low concentrations of theophylline progressively increase cell growth (by activating the td intron) while concentrations of theophylline above 2 mM progressively decrease cell growth (although levels of growth are still well above background).

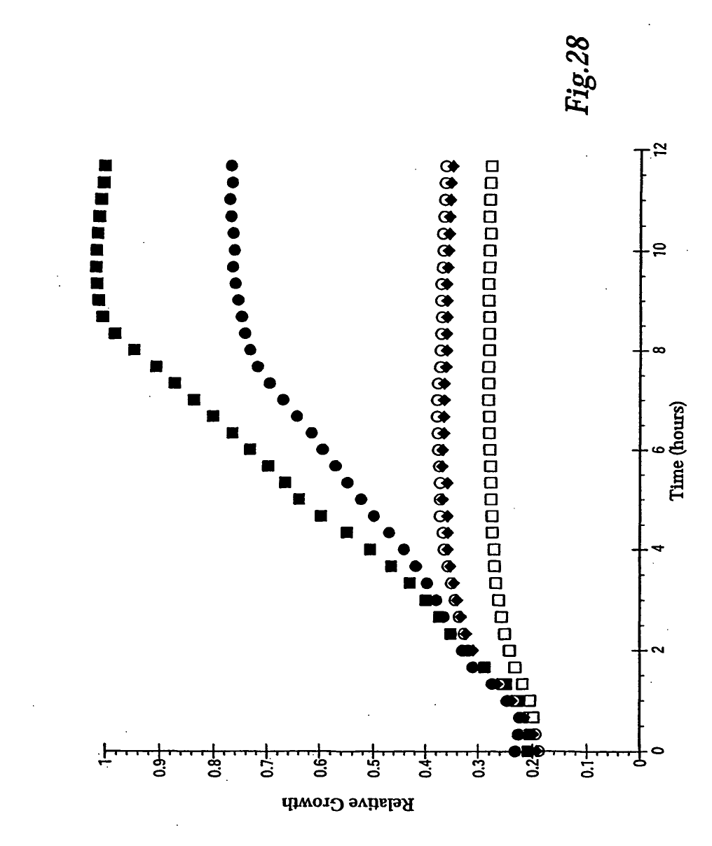

The presence of endogenous flavins made it difficult to examine effector specificity in vivo, and a new series regulatable, catalytically active nucleic acids were constructed in which the anti-theophylline binding sequence was mutated to bind 3methylxanthine (3MeX2P6). These variants proved to be responsive to 3-methylxanthine both in vitro and in vivo (FIG. 28). However, the variants were no longer responsive to theophylline, nor were they responsive to a variety of other analogues, including caffeine, 1-methylxanthine, 7-methylxanthine, 1,3-dimethyl urilic acid, hypoxanthine, xanthine, and theobromine.

These results indicate that theophylline regulates intron splicing in vivo. Next, mRNA was isolated from E. coli treated in the presence or absence of theophylline, and RT-PCR was used to confirm the presence of spliced introns. For each of the introns known to be responsive to theophylline in vivo (Th2P6 and Th1P5) an increase in spliced mRNA is observed, while those introns not responsive to theophylline in vivo did not show an increase in the levels of spliced mRNA. An exception to this was Th1P6, which originally showed theophylline-dependent splicing in vitro and theophylline-dependent splicing in vivo. However, Th1P6 does not mediate theophylline dependent growth. The cellular mRNAs were extracted, cloned, and sequenced, and half of them appeared to use a cryptic splice site.

The ability to engineer regulatable, catalytically active nucleic acids to be responsive to effector molecules has numerous potential applications. For example, it may be used in conjunction with new gene therapies in which patients rely upon drugs that differentially activate gene expression, rather than having to rely upon a set level of endogenous expression of an introduced gene. Similarly, it may be used with effector dependent splicing to more finely monitor gene expression in vivo. A drug that localized to particular organs, cells, or organelles, and splicing of the nucleic acid could be monitored via a reporter gene such as, e.g., luciferase. Engineered introns introduced into reporter genes may be used in high-throughput, cell-based screening assays that monitor drug uptake or efficacy.

Materials and Methods. E. coli strains and growth media. E. coli strain C600ThyA::KanR was used for the plate assays and in vivo growth curves. INVaF (Invitrogen, Carlsbad, Calif.) was used for cloning and plasmid amplification. Bacterial starter cultures were grown in LB supplemented with thymine (50 mg/l). Screening for the td phenotype was done in minimal media supplemented with 0.1% Norit A-treated casamino acids (MM) and MM supplemented with thymine (50 mg/l) (MMT). Plates contained Bacto agar (1.5%). Ampicillin (50 mg/l) and kanamycin (100 mg/l) were added to all growth media.

Plasmid. The wild type plasmid pTZtdl304 (Myers et al., 1996) contains a 265 nucleotide derivative of the 1016 nucleotide wild type intron that maintains wild type activity (Galloway Salvo et al., 1990) with additional mutations of U34A which introduces a Spe I site and U976G which introduces an EcoRI site.

Construction of the td intron regulatable, catalytically active nucleic acids. The constructs were made using standard solid phase DNA synthesis, then were PCR-amplified and cloned into pTZtdl304 that contained a 265 nucleotide derivative of the 1016 nucleotide wild-type intron. This derivative also contained the mutations U34A, which introduces a Spe I site, and U976G, which introduces an EcoRI site. The parental P6 nucleic acid construct was generated by two overlapping oligos, Gp1Wt2 Gp1 Wt2.122 (GCC TGA GTA TAA GGT GAC TTA TAC TTG TAA TCT ATC TAA ACG GGG AAC CTC TCT AGT AGA CAA TCC CGT GCT AAA TTG TAG GAC TGC CCG GGT TCT ACA TAA ATG CCT AAC GAC TAT CCC TT) (SEQ ID NO:16); and

Gp1Wt3.129 (TAA TCT TAC CCC GGA ATT ATA TCC AGC TGC ATG TCA CCA TGC AGA GCA GAC TAT ATC TCC AAC TTG TTA AAG CAA GTT GTC TAT CGT TTC GAG TCA CTT GAC CCT ACT CCC CAA AGG GAT AGT CGT TAG) (SEQ ID NO:17). These oligonucleotides (100 pmol) were annealed and extended with AMV reverse transcriptase (Amersham Pharmacia Biotech, Piscataway, N.J.; 45 units) in AMV RT buffer (50 mM Tris-HCl, pH 8.3, 8 mM MgCl₂, 50 mM NaCl, 1 mM DTT) and dNTPs (200 μM) for 30 min at 37° C. The resulting double-stranded DNA was diluted 1:50 and amplified using primers Spe 1.24 (TTA TAC TAG TAA TCT ATC TAA ACG (SEQ ID NO:18); 0.4 μM) and EcoRI.24 (CCC GGA ATT CTA TCC AGC TGC ATG (SEQ ID NO:19); 0.4 μM) in PCR buffer (10 mM Tris-HCl, pH 8.3, 50 mM KCl, 1.5 mM MgCl₂, 0.1% Triton X-100, 0.005% gelatin), dNTPs (200 μM) and Taq DNA polymerase (Promega, Madison, Wis.; 1.5 units). The reactions were thermocycled 15 times at 94° C. for 30 sec, 45° C. for 30 sec, 72° C. for 1 min and then purified with a QIAquick PCR purification kit (Qiagen, Valencia, Calif.).

The PCR product was digested with Spe I (New England Biolabs, Beverly, Mass.; units) and EcoRI(50 units) in buffer (50 mM NaCl, 100 mM Tris-HCl, pH 7.5, 10 mM MgCl₂, 0.025% Triton X-100, 100 μg/ml BSA) at 37° C. for 60 min, purified, and cloned into Spe I/EcoRI digested pTZtdl304. The negative control and nucleic acid constructs were made as described except that Gp1Wt3.129 was replaced with oligonucleotides of the appropriate sequence:

| | |
|---|---|
| B11 GCC TGA GTA TAA GGT GAC TTA TAC TTG TAA TCT | (SEQ ID NO:20) |
| ATC TAA ACG GGG AAC CTC TCT AGT AGA CAA TCC CGT GCT AAA TGC CTA | |
| ACG ACT ATC CCT T, | |
| Th1P6 GGC TGA GTA TAA GGT GAG TTA TAG TTG TAA TGT ATG TAA | (SEQ ID NO:21) |
| AGG GGG AAG GTG TGT AGT AGA GAA TGG GGT GGT AAA TTA TAC CAG CAT | |
| CGT CTT GAT GCC CTT GGC AGA TAA ATG CCT AAC GAC TAT CCC TT, | |
| Th2P6 GCC TGA GTA TAA GGT GAC TTA TAC TTG TAA TCT ATG TAA | (SEQ ID NO:22) |
| ACG GGG AAC CTC TCT AGT AGA CAA TCC CGT GCT AAA TTG ATA CCA GCA | |
| TCG TCT TGA TGC CCT TGG CAG CAT AAA TGC CTA ACG ACT ATC CCT T, | |
| Th3P6 GCC TGA GTA TAA GGT GAC TTA TAC TTG TAA TCT ATC TAA | (SEQ ID NO:23) |
| ACG GGG AAC CTC TCT AGT AGA CAA TCC CGT GCA TAC CAG CAT CGT CTT | |
| GAT GCC CTT GGC AGG CCT AAC GAC TAT CCC TT, | |
| Th4P6 GCC TGA GTA TAA GGT GAC TTA TAC TTG TAA TCT ATC TAA | (SEQ ID NO:24) |
| ACG GGG AAC CTC TCT AGT AGA CAA TCC CGT GCT AAA TAT ACC AGC ATC | |
| GTG TTG ATG CCC TTG GCA GTA AAT GCC TAA CGA CTA TCC CTT, | |
| Th5P6 GCC TGA GTA TAA GGT GAC TTA TAC TTG TAA TCT ATC TAA | (SEQ ID NO:26) |
| ACG GGG AAC CTC TCT AGT AGA CAA TCC CGT ATA CCA GCA TCG TCT TGA | |
| TGC CCT TGG CAG CTA ACG ACT ATC CCT T, | |
| Th6P6 GCC TGA GTA TAA GGT GAC TTA TAC TTG TAA TCT ATC TAA | (SEQ ID NO:27) |
| ACG GGG AAC CTC TCT AGT AGA CAA TCC CGT GAT ACC AGC ATC GTC TTG | |
| ATG CCC TTG GCA GCC TAA CGA CTA TCC CTT, | |
| Th1P5 TGA GTA TAA GGT GAC TTA TAC TAG TAA TCT ATC TAA ACG | (SEQ ID NO:28) |
| GGG AAG CTC TAT ACG AGC ATG GTC TTG ATG CCC TTG GGA GAG ACA ATG | |
| CCG TGG TAA ATT GTA GGA CTG CCC GGG TTC TAC ATA AAT GGG TAA CGA | |
| CTA TCC CTT, | |
| Th2P5 TGA GTA TAA GGT GAC TTA TAG TAG TAA TGT ATG TAA ACG | (SEQ ID NO:29) |
| GGG AAC CTA TAC CAG CAT CGT CTT GAT GCC CTT GGC AGA CAA TCC | |
| CGTGCTAAATTGTAGGACTGCCCGGGTTCTACATAAATGCCTAAC GAC TAT | |
| CCC TT, | |
| 3Mex2P6 GTA ATC TAT CTA AAC GGG GAA CCT CTC TAG TAG ACA ATC | (SEQ ID NO:30) |
| CCG TGC TAA ATT GAT ACC AGC ATCG GTC TTG ATG CCA TTG GCA GCA TAA | |
| ATG CCT AAC GAC TAT CCC TT, | |
| Th2P6.D GTA ATC TAT CTA AAC GGG GAA CCT CTC TAG TAG ACA ATC | (SEQ ID NO:31) |
| CCG TGC TAA ATT GAT ACC AGC ATC GTG TTG ATG CCC TTG GTT GCA TAA | |
| ATG CCT AAC GAC TAT CCC TT, | |
| FMN1P6 GCC TGA GTA TAA GGT GAG TTA TAC TTG TAA TCT ATC TAA | (SEQ ID NO:32) |
| ACG GGG AAC CTC TCT AGT AGA CAA TCC CGT GCT AAA TTA GGA TAT GCT | |
| TCG GCA GAA GGA TAA ATG CCT AAC GAC TAT CCC TT, and | |
| FMN2P6 GCC TGA GTA TAA GGT GAC TTA TAC TTG TAA TCT ATC TAA | (SEQ ID NO:33) |
| ACG GGG AAC CTC TCT AGT AGA CAA TCC CGT GCT AAA TTG AGG ATA TGC | |
| TTC GGC AGA AGG CAT AAA TGC CTA ACG ACT ATC CCT T. | |

In vitro transcription. The introns were PCR-amplified with 5′ le (GAT AAT ACG ACT CAC TAT AAT GGC ATT ACC GCC TTG T) (SEQ ID NO:34) and GM24 (GCT CTA GAC TTA GCT ACA ATA TGA AC) (SEQ ID NO:35) in 25 μl reactions under the conditions stated above and cycled 20 times. A portion of the reaction (5 μl) was run on a 3% agarose gel and the PCR product band was stabbed with a pipette tip. The agarose plug was added to a fresh PCR reaction (100 μl) and cycled 15 times; DNA was purified using a QIAquick kit and quantitated. The PCR product (2 μg in 50 μl) was added to an in vitro transcription reaction containing Ampliscribe T7 RNA polymerase (Epicentre), RNase inhibitor (GIBCO BRL, Rockville, Md.; 5 units), low Mg²⁺ buffer (30 mM Tris-HCl, pH 8, 7.5 mM DTT, 4.5 MM MgCl₂,1.5 mM spermidine), UTP (1.25 mM), ATP (2.5 mM), GTP (2.5 mM), CTP (7.5 mM) and αP³²-labeled UTP (NEN, Boston, Mass.; 20 μCi; 3000 mCi/mmol), and incubated at 37° C. for 2 h. DNase (GIBCO BRL, 295 units) was added and the reaction was incubated at 37° C. for an additional 30 min. The RNA was purified using Centri-Sep columns (Princeton Separations, Adelphia, N.J.) and quantitated.

In vitro splicing assays. The assays were preformed by heating the RNA (500 nM) in H₂O to 70° C. for 3 min then transferring to ice for 1 min. Splicing buffer (20 mM Tris-HCl, pH 7.5, 100 mM KCl, 3 mM MgCl₂), effector (Theophylline (1.5 mm) or FMN (1 mM)) was added and the reactions were incubated on ice for an additional 15 min. At this time a 4.5 μl aliquot was removed for a zero time point and quenched with 5 μl stop dye (95% formamide, 20 mM EDTA, 0.5% xylene cyanol, and 0.5% bromophenol blue). GTP (50 μM) was added to the remaining reaction (5 μl total volume) to start the splicing reaction. The reaction was incubated at 37° C. for 30 min and then terminated with stop dye (5 μl). The reactions were heated to 70° C. for 3 min and 5 μl was analyzed on an 8% denaturing polyacrylamide gel. The gel was dried, exposed to a phosphor screen and analyzed using a Molecular Dynamics Phosphorimager (Sunnyvale, Calif.).

The reaction volumes were increased for the rate determination assay. Aliquots were taken at intervals between 0 min and 30 min and terminated with stop dye. The reactions were analyzed as described above.

In vivo plate assay. The plasmids containing the various group I constructs were transformed into chemically competent C600ThyA::Kan^{R} cells and grown in LB with kanamycin overnight. A small aliquot (3 μl) of overnight cell culture was mixed with effector (theophylline (7.5 mM) or FMN (10 mM)) or H₂O, spotted on plates, and grown overnight at 37° C. As a positive control, all constructs were also plated on minimal media plates with thymine (MMT) and assayed for viability.

In vivo growth curves. Cells grown overnight in LB were diluted 1:100 in MM containing either theophylline, caffeine, 3-methylxanthine or no effector, and analyzed on a Microbiology Workstation Bioscreen C (Labsystems, Inc., Franklin, Mass.).

RT-PCR analysis. RNA was isolated from an overnight culture using a MasterPure RNA purification kit (Epicentre, Madison, Wis.) and amplified by RT-PCR using primers Tle and GM24 following the protocol provided for Tth polymerase. The products were separated and analyzed on a 3% agarose gel.

FIG. 25 shows the theophylline-dependent td group I intron constructs of the present invention. The FIG. 25(a) shows the predicted secondary structure and tertiary interactions of the 265 nucleotide deletion construct of the td intron. The intron is in uppercase and the exons are in lower case letters. The 5′ and 3′ splice sites are indicated by arrows. The P4-P6 domain is boxed. FIG. 25(b) shows the B11 construct based on the A85-863 deletion mutant of the td intron, which shows no activity at low Mg²⁺ (3 mM) in vitro or in vivo. An anti-theophylline aptamer, highlighted in gray, was substituted for the P6a stem of the intron in constructs Th1P6, Th2P6, Th3P6, Th4P6, Th5P6 and Th6P6, and for the P5 stem in constructs ThIP5 and Th2P5. Mutations in the anti-theophylline aptamer are boxed in black for constructs MeX2P6 and Th2P6.D. The C-to-A mutation in MeX2P6 changes specificity from theophylline to 3-methylxanthine. The A-to-U and C-to-U mutations in Th2P6.D abolished theophylline-binding.

The group I nucleic acids by theophylline was also demonstrated. The splicing activity of the parental, B11, Th1P6, Th2P6 and Th1P5 intron constructs in the presence and absence of 1.5 mM theophylline using autoradiography in which the following products were identified: LI, linear intron; Cl, circular intron; E1-E2, exon 1-exon 2 ligation product; Crp, cryptic ligation product; pre-mRNA, and unspliced mRNA.

FIG. 26 shows the design of an FMN-dependent td nucleic acid intron splicing construct. An anti-FMN aptamer, highlighted in gray, was substituted for the P6a stem in constructs FMN1P6 and FMN2P6. In vivo splicing activity was demonstrated on agar plates. The parental, B11 and theophylline constructs were spotted in the presence and absence of 7.5 mM theophylline on minimal media (MM), while the parental, B11 and FMN constructs were spotted in the presence and absence of 5 mM FMN.

Theophylline-dependent in vivo growth was assayed and quantitated. FIGS. 27(a), 27(b) and 27(c) show the relative growth curves are shown for C600:ThyA cells containing either Th2P6 (a) and Th1P5 (b) in the presence (0) and absence (0) of 0.5 mM theophylline or 0.5 mM caffeine (0). Parental (0) and B11 (0) controls were grown in the 0.5 mM theophylline for comparison. Plots are standardized to the growth of cells containing the parental intron. Each point represents the average of three replicate growth curves. FIG. 27(c) shows the extent of growth at 12 h for parental, Th2P6 and Th1P5 introns over a range of theophylline concentrations. Background growth (B11) has been subtracted, and results are standardized to parental growth with no theophylline.

FIG. 28 shows the 3-Methylxanthine dependent in vivo growth. Relative growth curves are shown for C600:ThyA cells containing 3MeX2P6 in the presence (0) and absence (0) of 1 mM 3-methtyxanthine or 1 mM theophylline (0). Parental (0) and B11 (0) controls were also grown in 1 mM 3-methylxanthine. Plots are standardized to parental growth. Each point represents the average of three replicate growth curves. To shows the splicing of introns in vivo, RT-PCR analysis of whole cell RNA was conducted. Bands corresponding to spliced and unspliced mRNAs were identified. Samples was seeded with RNA from cells grown in the absence of theophylline and compared with samples seeded with RNA from cells grown in the presence of 0.5 mM theophylline.

### EXAMPLE 6: DETECTION OF A DIVERSE SET OF ANALYTES USING ARRAYED RIBOZYME LIGASES

Several catalytic RNAs have been shown to be amenable to engineering. In several cases, a particular ribozyme scaffold can be evolved and engineered to respond to a wide variety of effectors. These properties give regulatable, catalytically active nucleic acids, tremendous potential in the field of molecular diagnostics. The engineering of the hammerhead ribozyme can yield variants that are allosterically regulated by a variety of ligands (Koizumi, M.; Kerr, J. N.; Soukup, G. A.; Breaker, R. R. Nucleic Acids Symp Ser., 1999, 42, 275-27). In addition, several of these allosteric hammerhead variants have in turn been used to assemble a ribozyme array able to detect a variety of small molecules.

In order to demonstrate the utility of ribozyme ligases in multiplexed, multiple analyte assays, a series of ligases developed by the inventors (described hereinabove) were used in an array. Notably, the array can detect a diverse range of biologically relevant analytes: small-molecules, nucleic acid, a protein and a peptide may be assayed in solution.

Regulatable ligase variants were evolved starting with a small ribozymc ligase, L1, which was initially selected from a random sequence pool. The activity of this ribozyme was found to be dependent upon the 3′ primer used in the selection, increasing the ribozyme's activity up to 10,000 fold in its presence. Additional L1 variants have been designed or selected to respond to small-molecules (ATP, FMN, theophylline), proteins (lysozyme), and peptides (Rev).

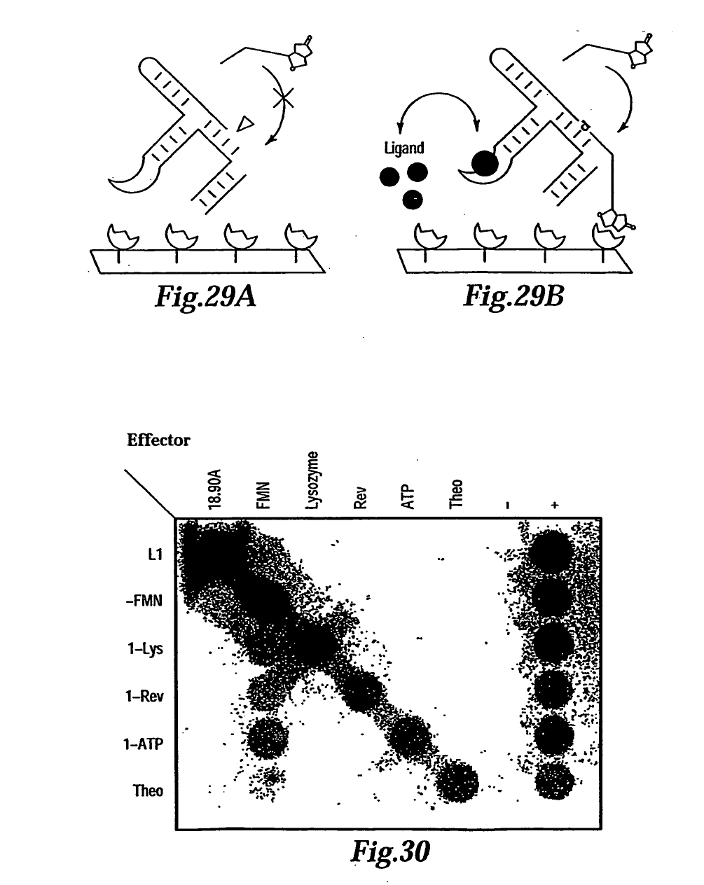

As an initial test of the ability of this ensemble of regulatable, catalytically active nucleic acids to function in a multiplexed assay, a simple scheme was developed for monitoring the self-attachment of the ligases to 96-well plates. By virtue of a biotinylated substrate, ligation of radio-labeled ribozymes in response to a given analyte can be monitored by quantitating the fraction immobilized in streptavidin coated polystyrene plates (FIG. 29).

A typical regulatable, catalytically active ligase array is depicted in FIG. 30. All the regulatable, catalytically active nucleic acids used (rows) were tested against the corresponding set of ligands (columns). The diagonal represents a positive reaction between an regulatable, catalytically active nucleic, acids and its cognate ligand. All regulatable, catalytically active nucleic acids were also tested for activity in complex mixtures (‘+’ column, mixture of all 6 ligands), as well as inactivity in the absence of effector (‘−’ column). For the most part, there is extremely high specificity between a particular regulatable, catalytically active nucleic acids and its cognate ligand. All of the regulatable, catalytically active nucleic acids retained activity in the context of a complex mixture. Note the cross-reactivity of L1-ATP with flavin mononucleotide (FMN), which may be due to chemical similarity between FMN and ATP. The array depicted in FIG. 30 is the ‘positive’ image of a typical assay, the supernatant removed following an assay was transferred to a separate plate for the quantitation of background and unligated 10 species.

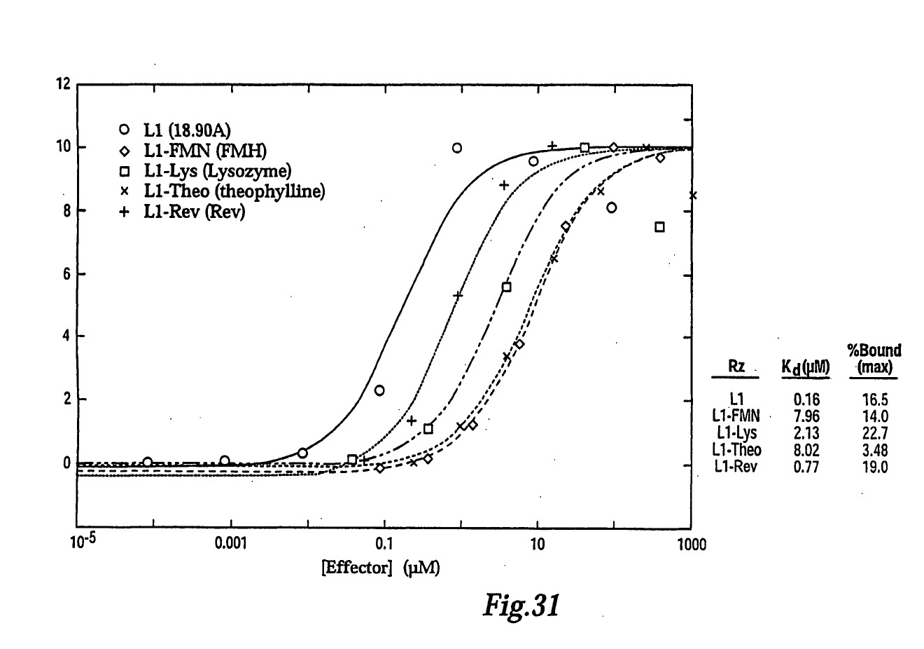

In order to better characterize individual aptazymes' properties in the context of an array, their ability to carry out ligation to a plate-bound substrate was monitored in response to ligand concentration (FIG. 31). Aptazymes (rows) were assayed in array format against the corresponding set of analytes (columns). Many of the aptazyme's activities are similar to values calculated previously. All of the ribozymes assayed displayed response characteristics with Kds in the high nM to low μM range.

FIG. 29 shows a schematic of ribozyme ligase array. In FIG. 29(a), the absence of analyte, the ribozyme is unable to catalyze the ligation of biotinylated substrate, and remains in the supernatant. In FIG. 29(b), analyte concentrations are high enough to cause ligation result in the self-attachment of a tagged substrate, which is then immobilized to streptavidin-coated 96-well plates.

FIG. 30 shows the results of a regulatable, catalytically active ligase array. Regulatable, catalytically active nucleic acids and effector pairs are assayed in array format; the ‘positive’ plate is pictured. The diagonal represents a positive reaction between a ribozyme and its cognate ligand.

FIG. 31 shows the titrations of individual allosteric ribozyme ligases. Response curves for five individual aptazymes are calculated. Normalized counts are plotted against cognate effector concentration (e.g. L1-FMN activity vs. [FMN]). Kd's are calculated by fitting data to a simple saturation curve (y=(m1*m0)/(Kd+m0)). The maximum percentage bound to the ‘positive’ plate is reported to illustrate the extent of ligation over the time allotted.

Sequences. Sequences for L1, L1-ATP, L1-FMN, and L1-theophylline have been published previously, while L1-Rev was recently selected: (SEQ). The 5′ primer used in PCR amplification incorporates a T7 promoter, while the T primer is universal for all templates.

RNA Preparation. Individual ribozymes were generated by standard in vitro transcription reactions containing 500 ng of PCR product, Tris-HCl, DTT, each of the four ribonucleotides, and 50 U of T7 RNA Polymerase. Following gel purification, the RNAs were eluted in water, precipitated and resuspended in water.

Aptazyme Array and Titration of Individual Aptazymes. Arrayed aptazyme assay were carried out by first annealing 100 pmol of ribozyme with 120 pmol of 18.90A (5, GCGACTGGACATCACGAG 3) (SEQ ID NO:36). Following addition of buffer (30 mM Tris-HCl, pH 7.5, 50 mM NaCl, 60 mM MgC1₂),120 pmol of substrate (S28A-biotin, 5′biotin-AAAAAAAAAAAAAAAAAAAAAAugcacu 3′, (SEQ ID NO:25) ribonucleotides in lowercase) was added. The reaction mixture was scaled to accommodate multiple aliquots for each corresponding well of the array. After aliquotting 50 μl into each well of an 96-well PCR plate (MJ Research), 50 μl of ligand in buffer was added. Ligand concentrations for FIG. 29 were: 1 μM 18.90A, 0.5 mM flavin mononucleotide (FMN), 5 μM lysozyme, 1 μM Rev peptide, 1 mM ATP, and 1 mM theophylline.

Reactions were incubated at 25° C. for 4 h, followed by the addition of 20 μl of 0.5 M EDTA. Reactions were then transferred to Hi-Bind streptavidin coated polystyrene plates (Pierce). Plates were again incubated at room temperature for 1 h, followed by the transfer of supernatant to a plain polystyrene 96-well plate. Wells in the Hi-bind plates were washed three times with buffer (30 mM Tris-HCl pH 7.5, 100 mM NaCl, 0.1% SDS, 7 M urea), followed by a rinse in TE (10 mM Tris-HCl, pH 7.5, 1 mM EDTA). Assays were quantitated by exposure to Phophorimager plates followed by analysis with ImageQuant software (Molecular Dynamics). Titrations (FIG. 31) were carried out essentially as described previously, with ligand titrated in a range a concentration.

### EXAMPLE 7: BIOSYNTHETIC APPL1CATIONS OF RCANAS

RCANAs according to the invention can be used as regulatory elements to control the expression of one or more genes in a metabolic pathway. RCANAs can also be used as regulated selectable markers to create a selective pressure favoring (or disfavoring) production of a targeted bioproduct. Furthermore, RCANAs can be used to control the production of a natural product in a biological host.

### A. Use of RCANAs as Regulatory Elements

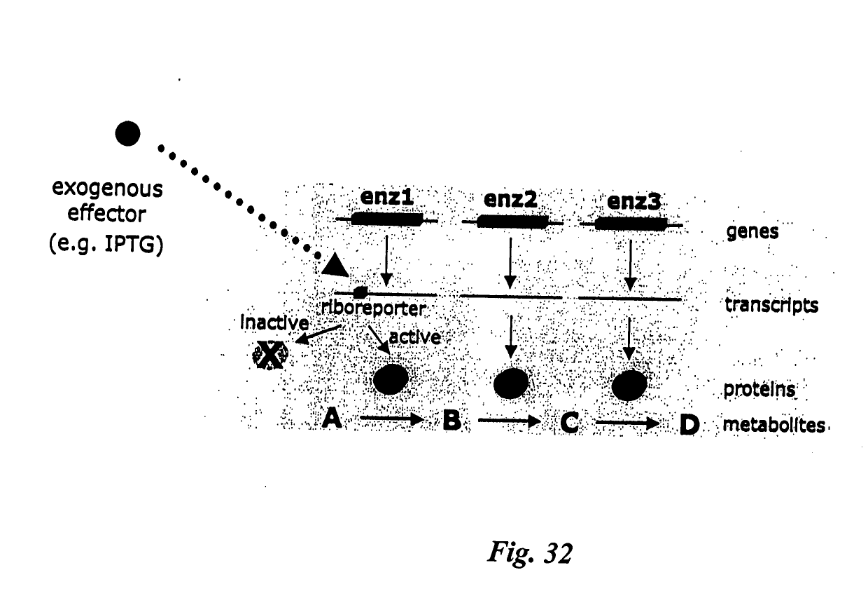

RCANAs can be used to control the expression of host genes involved in biosynthetic processes both ex vivo and in vivo. For in vivo applications, including intracellular applications, the RCANA is used to alter gene expression within a host organism. As shown in FIG. 32, effector-sensitive RCANAs can be embedded into RNA transcripts encoding biologically active polypeptides, e.g., enzymes, that participate in a biochemical metabolic pathway. Protein enzymes direct conversion of a precursor metabolite (A) through a number of biological transformations in to a desired end-product (D).

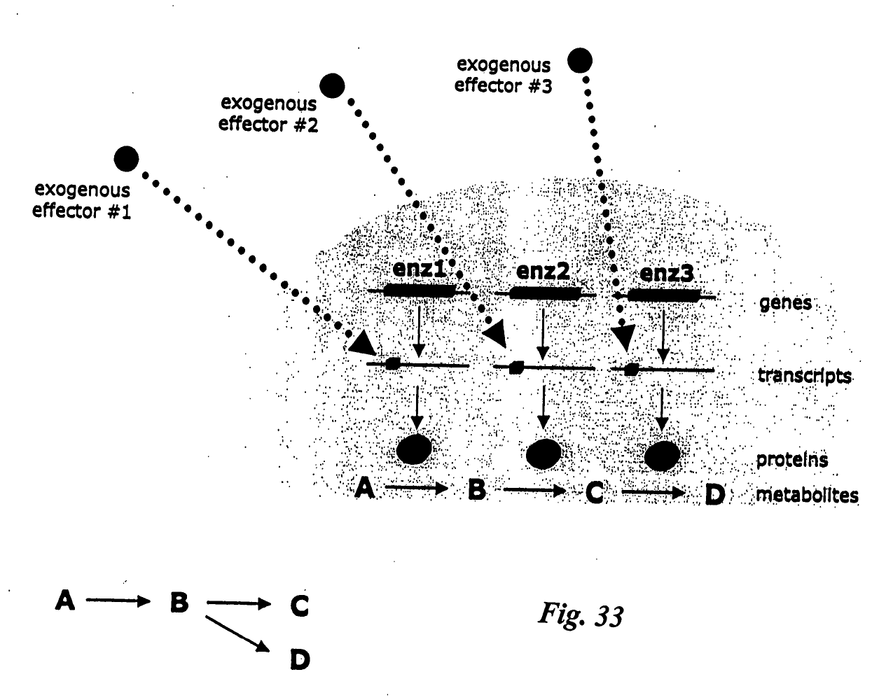

Effector Control. RCANAs can be engineered to respond to effector molecules, e.g., small xenobiotic molecules, in order to exert exogenous control of RCANA-mediated gene expression. Effector-sensitive RCANAs can be used alone (FIG. 32) or in combination (FIG. 33) to affect RCANA-mediated control of gene expression at a select point(s) in a biochemical metabolic pathway. The methods used to make effector-mediated RCANAs are well know in the art, e.g., EP97954396.6 and U.S. Ser. No. 97/24158.

As shown in FIG. 32, effector-senitive RCANA may be engineered into the gene encoding the polypeptide that carries out the first committed step in a biochemical metabolic pathway. In the absence of effector the enzyme, the expression of which is regulated by the effector-sensitive RCANA, is not produced, thus blocking the target biochemical metabolic pathway. For example, effector binding to an effector-sensitive self-splicing RCANA embedded within an enzyme transcript would activate its self-excision and thereby remove a premature stop codon that normally prevents translation.

As illustrated in FIG. 33, effector-RCANAs can be inserted into two or more enzymes in a biochemical metabolic pathway. Varying the concentrations of the effectors can be used to change the expression of effector-regulated RCANAs. Consequently, the flux of metabolite(s) through the biochemical metabolic pathway may altered.

RCANA-mediated gene expression can modulate the production of a target metabolite or the production of intermediate metabolites needed for the synthesis of a target metabolite. Alternatively, RCANA-mediated gene expression may effect the timing of bioproduct synthesis with respect to the growth of a cellular host. Accordingly, the use of RCANA-mediated control of gene expression in a biochemical metabolic pathway can yield an improved or optimized production of a metabolite/bioproduct.

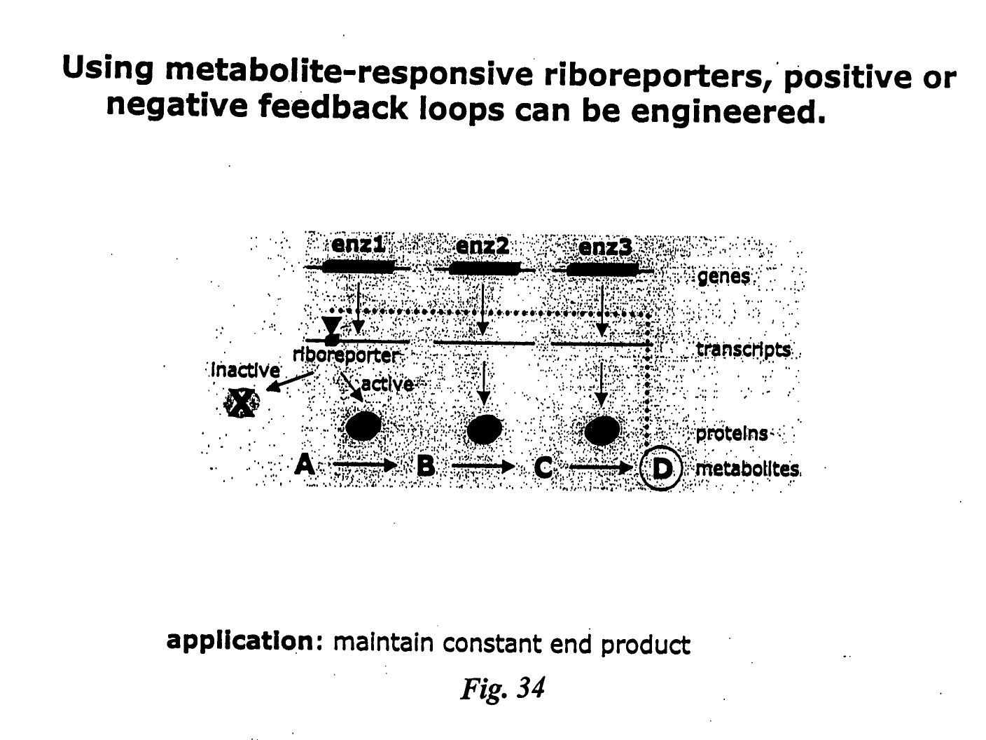

Enzyme feedback control. The use of RCANAs as regulatory elements is a flexible technology with many variations. Indeed, for different applications of RCANAs, the nature of the target molecule and the signal generated from RCANA-mediated catalysis will vary. For example, RCANAs can be designed to detect the metabolite produced in the metabolism of a biological molecule. As shown in FIG. 34, metabolite-responsive RCANAs can be used to create either a positive feedback loop or a negative feedback loop to control a biochemical metabolic pathway.

In a negative feedback loop, the metabolite-sensitive RCANA is configured to block expression of the target enzyme upon activation of the RCANA, e.g., using a self-cleaving effector-sensitive RCANA that catalyzes the degradation of the target RNA transcript in response to accumulation of end-product. In turn, metabolite-mediated degradation of RCANA-controlled target RNA transcripts would lead to reduced end-product synthesis via effects of lowered target enzyme level. It follows that, metabolite-responsive RCANAs can be used to maintain a constant rate of production of the end-product of a biochemical metabolic pathway.

In a positive feedback loop, the metabolite-sensitive RCANA is configured to activate expression of the target enzyme upon activation of the RCANA, e.g., using a self-splicing intron that catalyzes its removal from the target RNA transcript. In host cells carrying the metabolite-sensitive RCANA, the metabolic pathway will remain off for an extended initial period. Low-level flux through the metabolic pathway will ultimately cause accumulation of the end-product leading to a metabolite-sensitive RCANA activation and elevated end-product synthesis due to RCANA-mediated up-regulation of end-product synthesis.

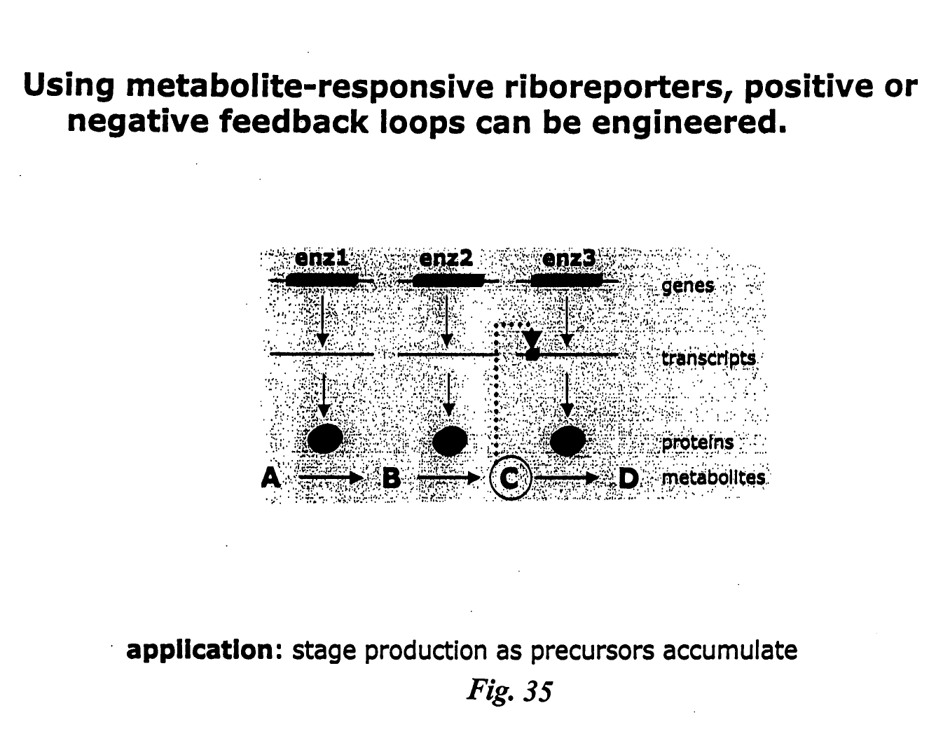

Iniermediate-induced control. As shown in FIG. 35, an effector-sensitive RCANA activated by an intermediate in a biochemical metabolic pathway can control expression of a downstream enzyme in the same pathway. As a result, synthesis of the end-product is delayed until intermediates in the pathway accumulate. This approach may be especially useful where end-product is toxic to the host organism. Non-toxic intermediates can be built up, and once available, the synthesis completed upon expression of the final enzymes in the biochemical metabolic pathway.

### B. Use of RCANAs as Regulated Selectable Markers

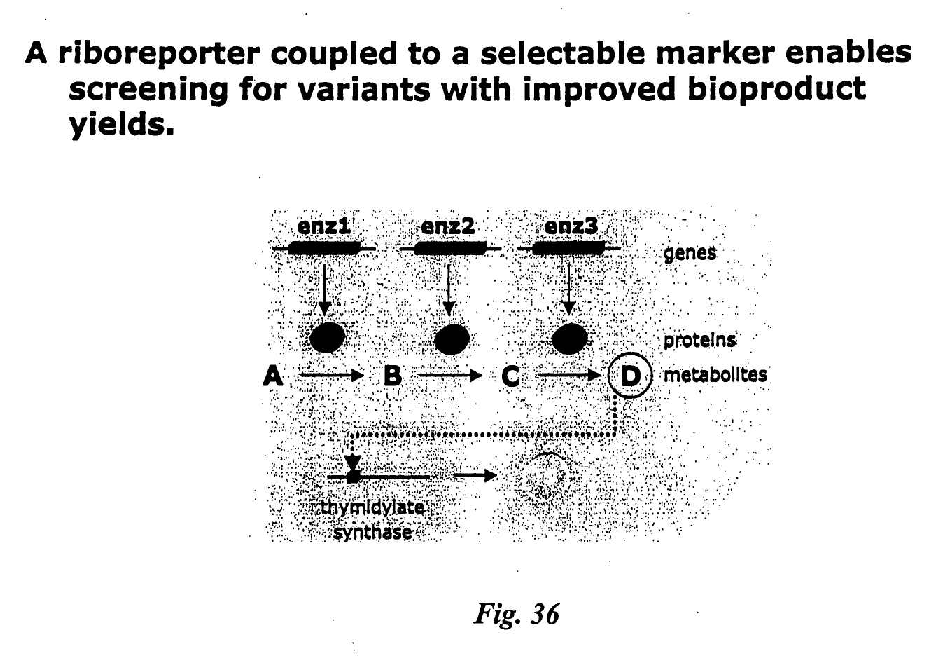

Variants of host cells/organisms with desired characteristics, e.g., optimal synthesis of a bioproduct, can be isolated employing a metabolite-sensitive RCANA-based selection strategy. As shown in FIG. 36, a metabolite-sensitive RCANA responsive to the desired metabolite is designed such that catalytic activity is triggered by high concentrations of the effector metabolite. The metabolite-sensitive RCANA is embedded within a gene encoding a reporter or a selectable marker, (e.g., green fluorescent protein (GFP), thymidylate synthase, or β-lactamase) so that the reporter/selectable marker is expressed only in the presence of the effector metabolite. The metabolite-sensitive RCANA may be a self-splicing group I intron that promotes self-excision from the selectable marker transcript to allow translation of a full-length protein.

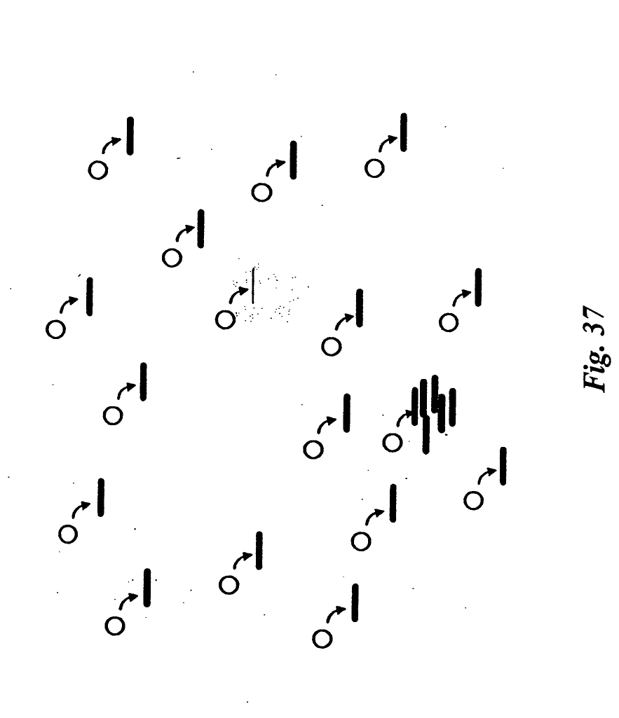

As shown in FIG. 37, following the design and engineering of the metabolite-sensitive RCANA-bearing selectable marker, the construct is introduced into a genetically diverse population of host cells/organisms. The host strain may be an expression library of mutant forms of a metabolic enzyme involved in bioproduct synthesis. For screening applications, the population of host cells/organisms is then grown under conditions allowing selection of cells based on expression of the metabolite-sensitive RCANA-bearing selectable marker, e.g., GFP-expressing cells may be sorted using a fluorescence-activated cell sorter. For selection applications, the population of selectable marker-bearing cells are grown under conditions that couple survival/growth rate to expression of the selectable marker, e.g., growth in minimal media lacking thymidine (using thymidylate synthase as a selectable marker) or media supplemented with ampicillin (using APR as a selectable marker). Under these conditions, high level synthesis of the desired metablite is required for cell survival, leading to the enrichment of the most efficient producers of this product.

### C. Use of RCANAs as Biosensors

Effector-sensitive RCANAs can be used to accurately monitor natural product formation in real-time. That is, an effector-sensitive RCANA can monitor the concentration of a natural product as it is produced, either directly in vivo or ex vivo, e.g., following cell host lysis. Effector-sensitive RCANAs can be designed to detect a wide range of environmental conditions. Variant host strains engineered to contain effector-sensitive RCANAs can be tested for the synthesis of a natural product. Accordingly, effector-sensitive RCANAs can be used to define the conditions or variant host strains for optimal synthesis of a natural product.

An effector-sensitive RCANA can be used to infer the concentration of a targeted bioproduct in host cells. The RCANA-mediated signal measured in varying growth/incubation conditions, e.g., growth temperature, media composition, cell density at induction, oxygenation level, time of induction, can be measured to define optimal growth of a test host expressor organism. The growth/incubation conditions that yield the strongest RCANA-mediated signal from a test host organism can be identified as the conditions that optimize the synthesis/accumulation of a target bioproduct.

A gene encoding an effector-sensitive RCANA, together with elements required for its intracellular transcription, is engineered into the host cells (carried on either a host chromosome or as part of an cxtrachromosomal vector). Alternatively, the effector-sensitive RCANA is transfected into cells or contacted with extracts following lysis. The effector-sensitive RCANA is configured such that its catalytic activity may be easily monitored by one of the methods outlined below:

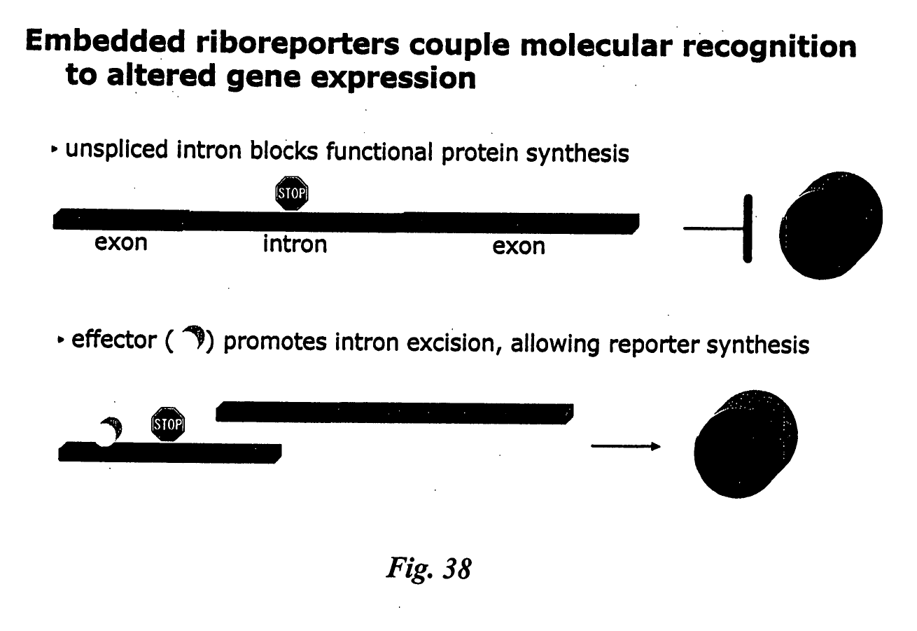

1. The effector-sensitive RCANA is expressed together with a gene encoding a reporter protein that generates a visible signal directly, e.g., GFP, or as a result of the catalytic transformation of a chromogenic or fluorogenic substrate, e.g., β-lactamase. As shown in FIG. 38, if the RCANA is active, it induces modifications to the reporter protein transcript that either allows or blocks the ability of the transcript to generate an active form of the protein. Modifications to the transcript include, e.g., splicing (the preferred embodiment), self-cleavage, circularization, capping, and editing. The RCANA-mediated modification to the target transcript need not result in ligation of the nucleic acid sequence spanned by the RCANA, e.g., endonucleolytic RCANA. The activation of the RCANA does, however, alter the stability, translational efficiency, or subcellular localization of the target transcript, or a combination thereof.

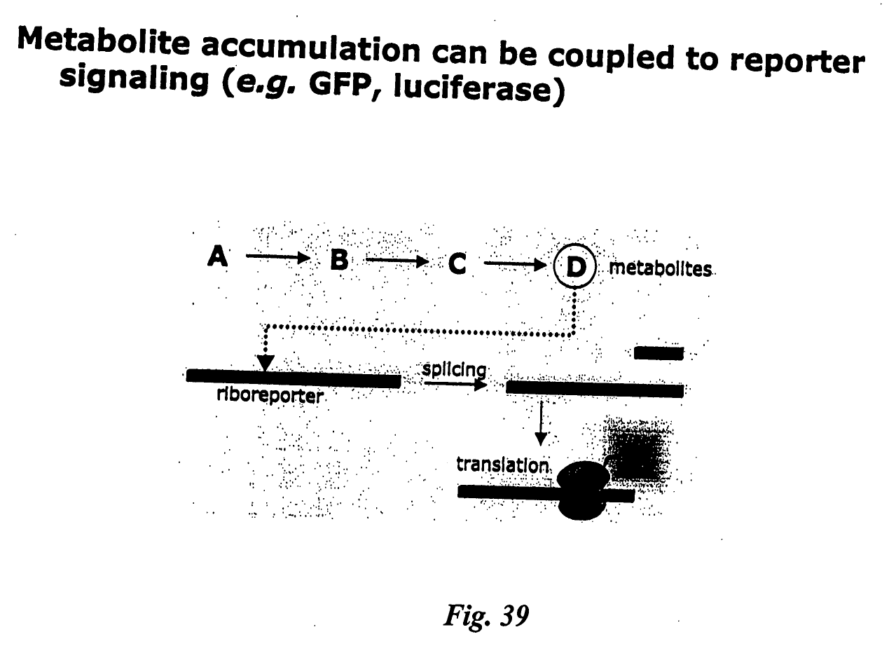

FIG. 39 shows the use of an effector-sensitive RCANA as an in vivo sensor. As shown in the Figure, an in vivo sensor system that uses an effector-sensitive RCANA can reveal levels of a metabolite (D). An RCANA sensitive to metabolite D is embedded in an RNA encoding a reporter polypeptide. Activation of the metabolite-sensitive RCANA yields, e.g., the splicing of the RCANA and the removal of the RCANA from the target transcript which, in turn, allows for the translation of the reporter polypeptide.

2. The effector-sensitive RCANA is provided with a chromomeric or fluorogenic substrate that it acts upon. The substrate can be administered directly to effector-sensitive RCANA-containing host cells, e g, a fiuorophore-quencher-bearing oligonucleotide substrate may be transfected into cells. Alternatively, host cells can be lysed and the substrate mixed with the resulting cell extract in order to yield a signal.

3. The effector-sensitive RCANA is configured such that catalysis changes the ability of the effector-sensitive RCANA to generate a nucleic acid product that can be detected via an enzymatic amplification reaction, e.g., reverse transcriptase/PCR or rolling circle amplification. For example, the RT/PCR assay is carried out using a ligase RCANA designed to catalyze self-circularization. Using a pair of outwardly-directed oligonucleotide primers corresponding to the ends of the ribozyme, only circularized molecules will generate an amplifiable signal.

All publications mentioned in the above specification are hereby incorporated by reference. Modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described compositions and modes of carrying out the invention that are obvious to those skilled in molecular biology or related arts are intended to be within the scope of the following claims.

What is claimed is:

1. A method of regulating production of a product in a cell, comprising:
inserting into a gene that produces said product or regulates the production of said product in said cell a regulatable catalytically active nucleic acid (RCANA), comprising a catalytic domain, which modifies a transcript to alter its coding potential, and a regulatory domain which recognizes an effector that alters the function of the catalytic domain;
contacting said regulatory domain with an effector thereby regulating production of said product.

2. The method of claim 1, wherein the concentration of the effector modulates the activity of the catalytic domain of said RCANA.

3. The method of claim 1, wherein the production of said product is fully inhibited.

4. The method of claim 1, wherein the production of said product is increased compared to a normal control level.

5. The method of claim 1, wherein the production of said product production is partially inhibited according to the concentration of the effector.

6. The method of claim 1, wherein the effector is the product.

7. The method of claim 1, wherein the effector is a feedback inhibitor of said gene.

8. The method of claim 1, wherein said product is produced in a metabolic pathway that is being regulated.

9. The method of clain 1, wherein said effector is an intermediate in a metabolic pathway.

10. The method of claim 1, wherein said RCANA blocks expression of said gene.

11. The method of claim 1, wherein said RCANA activates expression of said gene.

12. The method of claim 1, wherein said product is an intermediate of a metabolic pathway.

13. The method of claim 1, wherein said biological pathway is a metabolic pathway.

14. The method of claim 1, wherein the effector is endogenous to said cell.

15. The method of claim 1, wherein the effector is exogenous to said cell.

16. The method of claim 1, wherein the effector is selected from the group consisting of a protein, an enzyme, a protein pharmaceutical, a metabolite, a drug, a dye, a vitamin, a food additive, a chemical additive, a pesticide, an insecticide, a feed compound, and a waste product.

17. The method of claim 16 wherein the drug is selected from the group consisting of antibiotics, anticancer drugs, antifungals, cholesterol-lowering drugs, and immunosuppressants,

18. The method of claim 1, wherein the product is selected from the group consisting of a protein, an enzyme, a protein pharmaceutical, a metabolite, a drug, a dye, a vitamin, a food additive, a chemical additive, a pesticide, an insecticide, and a feed compound.

19. The method of claim 1, wherein said effector is an endproduct of a biosynthetic process.

20. A method of regulating a biological pathway in a cell, comprising:
inserting into a first gene that produces a first product or regulates the production of said first product in said biological pathway in a cell a first regulatable catalytically active nucleic acid (RCANA), comprising a catalytic domain, which catalyzes cleavage of the RCANA or excision of the RCANA from gene in which it is inserted followed by ligation ofthc gene at 5′ and 3′ ends of cleavage site, and a regulatory domain which recognizes an effector that activates a function of the catalytic domain;
inserting into a second gene that produces a second product or regulates the production of said second product in said biological pathway in said cell a second regulatable catalytically active nucleic acid (RCANA), comprising a catalytic domain, which catalyzes cleavage of the RCANA or excision of the RCANA from gene in which it is inserted followed by ligation of the gene at 5′ and 3′ ends of cleavage site, and a regulatory domain which recognizes an effector that activates a function of the catalytic domain;
contacting said first regulatory domain with a first effector thereby regulating production of said first product, and contacting said second regulatory domain with a second effector thereby regulating production of said second product.

21. The method of claim 20, wherein the combination of said first and second effectors control the flux of metabolites through the biological pathway.

22. The method of claim 20, wherein said biological pathway is a biosynthetic pathway.

23. The method of claim 20, wherein said biological pathway is a metabolic pathway.

24. The method of claim 20, wherein the biological pathway is fully inhibited.

25. The method of claim 20, wherein the biological pathway is partially inhibited according to the concentration of said first and second effectors.

26. The method of claim 20, wherein said first product is the second effector.

27. The method of claim 20, further comprising inserting into a third gene that produces a third product or regulates the production of said third product in said biological pathway in said cell a third regulatable catalytically active nucleic acid (RCANA), comprising a catalytic domain, which catalyzes cleavage of the RCANA, or excision of the RCANA from gene in which it is inserted followed by ligation of the gene at 5′ and 3′ ends of cleavage site, and a regulatory domain which recognizes an effector that activates a function of the catalytic domain.

28. The method of claim 20, wherein said first and second RCANAs block expression of said first and second gene.

29. The method of claim 20, wherein said first and second RCANAs activate expression of said first and second gene.

30. A method of screening a population of cells for a cell that produces a bioproduct, comprising:
inserting a regulatable catalytically active nucleic acid into a reporter gene in said population of cells, such that the regulatable catalytically active nucleic acid is regulated by said bioproduct;
wherein expression of said reporter gene indicates the production of said bioproduct a cell.

31. The method of claim 30, further comprising isolating said cell that produces said bioproduct.

32. The method of claim 30, wherein said reporter gene is green fluorescent protein, thymidylate synthase, or beta lactamase.

33. A polynucleotide that is regulated by a peptide comprising:
a regulatable, catalytically active polynucleotide, wherein the peptide interacts with the polynucleotide to affect its catalytic activity

34. A nucleic acid that is regulated by an effector comprising:
a regulatable, catalytically active nucleic acid, generated by the modification of at least one catalytic residue.

35. A nucleic acid comprising:
a gene; a regulatable, catalytically active nucleic acid inserted within the gene; wherein the presence of an effector causes the nucleic acid to catalyze a reaction.

36. A nucleic acid segment comprising:
a regulatable, catalytically active nucleic acid, selected from a pool of nucleic acids in which at least one of the catalytic residues has been randomized.

37. A regulatable, catalytically active nucleic acid segment comprising:
an effector domain; and a nucleic, acid catalyst domain in which one or more critical catalytic residues of the nucleic acid catalyst have been randomized; wherein the kinetic parameters of the catalytic domain are regulated by an effector that interacts with the effector domain.

38. A method of isolating a regulatable, catalytically active nucleic acid, comprising the steps of-randomizing at least one nucleotide in the catalytic domain of a catalytically active nucleic acid to create a nucleic acid pool; removing from the nucleic acid pool those nucleic acids that interact with the catalytic target of the catalytic domain; adding an effector molecule to the nucleic acids; and isolating those nucleic acids that interact with the catalytic target of the catalytic domain.

39. A method of isolating a regulatable, catalytically active nucleic acid having a catalytic and an effector domain, comprising the steps of-randomiziig at least one nucleotide in the catalytic domain of the nucleic acid to create a nucleic acid pool; removing from the nucleic acid pool those randomized nucleic acids that interact with the catalytic target of the catalytic domain; adding an effector to the nucleic acids; and isolating the nucleic acids that interact with the catalytic target of the catalytic domain.

40. A method of detection of a target using a regulatable, catalytically active nucleic acid comprising the steps of:
contacting the a regulatable, catalytically active nucleic acid with the target; and measuring the effect of the interaction between the a regulatable, catalytically active nucleic acid and the target.

41. A method of modifying a target using a regulatable, catalytically active nucleic acid comprising the steps of-providing a regulatable, catalytically active nucleic acid capable of target specific modification; and modifying the target under conditions that cause a regulatable, catalytically active nucleic acid-specific activity.

42. A method of selecting a regulatable, catalytically active nucleic acid, comprising the steps of-contacting a pool of nucleic acids, the nucleic acids having a catalytic and an effector domain, wherein at least one nucleotide in the catalytic domain of the nucleic acids has been randomized; removing from the nucleic acid pool those nucleic acids that interact with the catalytic target of the catalytic domain; adding an effector to the remaining nucleic acids; and isolating those nucleic acids that interact with the catalytic target of the catalytic domain; introducing the nucleic acids into a host cell; and measuring the catalytic activity of the nucleic acid upon exposure of the host cell to the effector.

43. A method of detecting a regulatable, catalytically active nucleic acid, comprising the steps of:
isolating a regulatable, catalytically active nucleic acid; creating a construct in which the nucleic acid is in position to regulate the expression of a reporter gene; introducing the construct into a host cell; and measuring the catalytic activity of the nucleic acid upon exposure of the host cell to an effector.

44. A method of modulating expression of a nucleic acid, the method comprising providing a polynucleotide that is regulated by a peptide, the polynucleotide comprising a regulatable, catalytically active polynucleotide, wherein the peptide interacts with the polynucleotide to affect its catalytic activity; and contacting the polynucleotide with the peptide, thereby modulating expression of a nucleic acid.

45. The method of claim 44, wherein the polynucleotide is provided in a cell.

46. The method of claim 44, wherein the cell is provided in vitro.

47. The method of claim 44, wherein the cell is provided in vivo.

48. The method of claim 44, wherein the cell is a prokaryotic cell.

49. The method of claim 44, wherein the cell is a eukaryotic cell.

50. A method of modulating expression of a nucleic acid, the method comprising the steps of-providing a nucleic acid that is regulated by an effector, the nucleic, acid comprising: a regulatable, catalytically active nucleic acid, wherein the regulatable, catalytically active nucleic acid molecule includes at least one modified catalytic residue; and contacting the nucleic acid with the effector, thereby modulating expression of a nucleic acid.
